(19) United States
(12) Patent Application Publication
(10) Pub. No.: US 2024/0141356 A1
(43) Pub. Date: May 2, 2024

(54) Driving Axon Regeneration by Activating STAT1 Signaling and cGAS-STING Pathway

(71) Applicant: The Hong Kong University of Science and Technology, Hong Kong (CN)

(72) Inventors: Kai LIU, Hong Kong (CN); Xu WANG, Hong Kong (CN); Chao YANG, Hong Kong (CN)

(21) Appl. No.: 18/466,254

(22) Filed: Sep. 13, 2023

Related U.S. Application Data
(60) Provisional application No. 63/406,717, filed on Sep. 14, 2022.

(51) Int. Cl. C12N 15/113; A61K 31/166; A61K 31/433; A61K 45/06; A61P 25/00; C12N 5/0793; C12N 9/22

(52) U.S. Cl. CPC C12N 15/1137; A61K 31/166; A61K 31/433; A61K 45/06; A61P 25/00; C12N 5/0619; C12N 9/22; C12N 2310/122; C12N 2310/20

(57) ABSTRACT

The subject invention pertains to a method for promoting axon regeneration in a subject with central nervous system injury. More specifically, the method comprises activating STAT1 signaling, cGAS-STING pathway, or a combination thereof by administering IFNγ and inhibiting the expression or function Protein Tyrosine Phosphatase Non-Receptor Type 2 (PTPN2) inhibitor; or administering a STING agonist.

### CROSS-REFERENCE TO RELATED APPLICATION

This application claims the benefit of U.S. Provisional Patent Application No. 63/406,717, filed Sep. 14, 2022, which is hereby incorporated by reference in its entirety including any tables, figures, or drawings.

### SEQUENCE LISTING

The Sequence Listing for this application is labeled “HKUS.181X.xml” which was created on Aug. 28, 2023 and is 33,483 bytes. The entire contents of the sequence listing is incorporated herein by reference in its entirety.

### BACKGROUND OF THE INVENTION

Neurotrauma in the adult mammalian central nervous system (CNS), such as spinal cord injury, leads to devastating and persistent neurological deficits, including paralysis. When axonal damage occurs without efficient axon regeneration, despite many neurons surviving the damage, neural networks become disconnected and paralysis results. In contrast, in the peripheral nervous system (PNS), the dorsal root ganglion (DRG) neurons can spontaneously regenerate their peripheral axons after nerve injury. Comparing the regeneration processes between the CNS and PNS has helped us understand why CNS axons fail to regenerate (Chandran et al., 2016; Hilton et al., 2022; Neumann and Woolf, 1999; Palmisano et al., 2019; Tedeschi et al., 2016; Weng et al., 2018; Yang et al., 2020). A leading working model is that regeneration failure in the CNS is a result of both the limited growth capability intrinsic to the CNS neurons as well as their extrinsic inhibitory environment (Fawcett, 2020; He and Jin, 2016; Mahar and Cavalli, 2018; Silver et al., 2014; Tedeschi and Bradke, 2017). Another important regulatory component of axon regeneration and neural repair is the innate immune response (Benowitz and Popovich, 2011), potentially linking remote neurons to signals from the lesion site. Studies in the PNS support the notion that distant nerve injuries elicit inflammatory responses not only in the local environment but also in remote neurons, even before immune cells infiltrate into the lesion site. For instance, upon injury, ciliary neurotrophic factor (CNTF) derived from Schwann cells contributes to cytokine production in DRG neurons (Hu et al., 2020). In addition to the environment-derived immune response, it has been reported that peripheral axotomy enhances neuronal SARM1 (Wang et al., 2018), axonal STAT3 (Ben-Yaakov et al., 2012), and neuronal STAT3 (Qiu et al., 2005) in DRG neurons, which is indicative of an intrinsic immune reaction in neurons after injury. Contrastingly in the CNS, optic nerve injuries increase Cntf gene expression only transiently in retinal ganglion cells (RGCs) (Smith et al., 2009), which is not sufficient for axon regeneration. Another study found that inducing inflammation within the retina by directly injecting zymosan, a fungal cell wall extract, can induce infiltration and activation of macrophages and neutrophils in the eye. The increased cytokines and growth factors produced by these activated immune cells were found to promote axon regeneration (Baldwin et al., 2015; Sas et al., 2020; Yin et al., 2003). Collectively, this evidence supports the hypothesis that the innate immune responses are tightly controlled in CNS neurons by inhibitory mechanisms. The identification of immune response inhibitors that contribute to regeneration failure will enable development of strategies to facilitate neural repair in CNS.

As a critical cytokine that regulates both innate and adaptive immunity against viruses and bacteria, IFNγ binds with the IFNGR1 and IFNGR2 heterodimers and functions via STAT signaling (Schroder et al., 2004). Recently, accumulating evidence has shown that IFNγ directly participates in neuronal development. Transient IFNγ treatment of human iPSC-derived neural progenitors increased neurite length (Warre-Cornish et al., 2020). During optic tract development, JAK2-STAT1 signaling drives the elimination of inactive synaptic connections in the brain (Yasuda et al., 2021). Moreover, animal studies have demonstrated that Ifng- or Stat1-knockout mice exhibit social deficits and abnormal neuronal connectivity (Filiano et al., 2016). However, the function of IFNγ during CNS trauma and regeneration is still under debate, largely because most studies have used approaches such as germline knockout or systemic ligand administration that cannot separate IFNγ signaling in neurons from non-neuronal cells. How neuronal IFNγ signaling and its downstream contributes to the neural repair process remains unknown.

The cyclic guanosine monophosphate-adenosine monophosphate (cGAMP) synthase (cGAS)-stimulator of interferon genes (STING) pathway has emerged as a key regulator between exogenous DNA recognition and type I interferon production (Ablasser and Chen, 2019). Once bound to exogenous DNA, the catalytic activity of cGAS leads to the production of 2′, 3′ cGAMP, a cyclic di-nucleotide as an innate agonist of STING. Then, the STING oligomer translocates from the ER to the Golgi apparatus and recruits tank binding kinase 1 (TBK1) and interferon regulatory factor 3 (IRF3). Phosphorylated IRF3 moves to the nucleus and activates the expression of type I interferon. In addition to defending against invading pathogens, neurodevelopment also requires activating the cGAS-STING pathways. STING deletion was found to reduce neuronal differentiation and leads to autistic-like behaviors in mice (Zhang et al., 2020). Recently, it has been reported that STING functions as a critical regulator of nociception through IFN-I signaling in DRG and STING agonists attenuate neuropathic pain induced by nerve injury or peripheral neuropathy (Donnelly et al., 2021), indicating its additional role beyond immune regulation. However, the role of the cGAS-STING pathway in axon regeneration remains unclear.

Thus, a novel method for axon regeneration is needed.

### BRIEF SUMMARY OF THE INVENTION

The subject invention pertains to novel compositions and methods that alter the coordination mechanism of neural innate immune responses for axon regeneration. More specifically, the subject invention alters the neural antiviral mechanism to promote axon regeneration in the central nervous system (CNS). In certain embodiments, the inhibition of the expression of protein tyrosine phosphatase non-receptor type 2 or the inhibition of the functioning of protein tyrosine phosphatase non-receptor type 2 increases IFNγ-STAT1 activity in retinal ganglion cells (RGCs) to promote axon regeneration after injury, independent of mTOR or STAT3. In certain embodiments, the methods comprise activating the cGAMP synthase (cGAS)-stimulator of interferon genes (STING) DNA sensing pathway to promote axon regeneration. In certain embodiments, the methods comprise administering IFNγ to a subject in need thereof, in which the IFNγ is locally translated in the injured peripheral axons and upregulates cGAS expression in Schwann cells and infiltrating blood cells to produce cGAMP, which promotes spontaneous axon regeneration.

In certain embodiments, protein tyrosine phosphatase non-receptor type 2 (Ptpn2) inhibition enhances CNS axon regeneration, and Ptpn2 inhibition combined with low-dose IFNγ injection enhances CNS axon regeneration. In certain embodiments, IFNγ can be administered in a composition at a concentration of about 0.1 μg/μl to about 0.2 μg/μl to a subject. In certain embodiments, about 2 μl of the composition comprising IFNγ can be administered locally to a subject. In certain embodiments, a Ptpn2 deletion amplifies IFNγ-STAT1 signaling. In certain embodiments, in the PNS, injury triggered IFNγ upregulates cGAS expression in Schwann cells and blood cells. In addition, in certain embodiments, the administration of cGAMP promotes spontaneous axon regeneration in the PNS. Advantageously, the subject invention can be used in methods of axon regeneration in both the CNS and PNS.

### BRIEF DESCRIPTION OF THE SEQUENCES

SEQ ID NO: 1: PCR primer: Ifit1 forward
SEQ ID NO: 2: PCR primer: Ifit1 reverse
SEQ ID NO: 3: PCR primer: Ifi204 forward
SEQ ID NO: 4: PCR primer: Ifi204 reverse
SEQ ID NO: 5: PCR primer: Mx2 forward
SEQ ID NO: 6: PCR primer: Mx2 reverse
SEQ ID NO: 7: PCR primers: Sprr1a forward
SEQ ID NO: 8: PCR primers: Sprr1a reverse
SEQ ID NO: 9: PCR primers: Gadd45a forward
SEQ ID NO: 10: PCR primers: Gadd45a reverse
SEQ ID NO: 11: PCR primers: Tubb6 forward
SEQ ID NO: 12: PCR primers: Tubb6 reverse
SEQ ID NO: 13: PCR primers: Apod forward
SEQ ID NO: 14: PCR primers: Apod reverse
SEQ ID NO: 15: PCR primers: Fst forward
SEQ ID NO: 16: PCR primers: Fst reverse
SEQ ID NO: 17: PCR primers: Camk1 reverse
SEQ ID NO: 18: PCR primers: Camk1 reverse
SEQ ID NO: 19: sgRNA sequence targeting Ptpn2
SEQ ID NO: 20: sgRNA sequence targeting Ptpn2
SEQ ID NO: 21: shRNA sequence targeting Ptpn2
SEQ ID NO: 22: Control shRNA
SEQ ID NO: 23: Ifnar1 shRNA
SEQ ID NO: 24: Ifnar2 shRNA
SEQ ID NO: 25: Ifngr1 shRNA
SEQ ID NO: 26: Ifngr2 shRNA
SEQ ID NO: 27: Stat1 shRNA
SEQ ID NO: 28: Pten shRNA
SEQ ID NO: 29: Ptpn2 shRNA
SEQ ID NO: 30: Ifng shRNA
SEQ ID NO: 31: Tubb6 shRNA
SEQ ID NO: 32: Camk1 shRNA
SEQ ID NO: 33: Apod shRNA
SEQ ID NO: 34: Fst shRNA
SEQ ID NO: 35: Control sgRNA
SEQ ID NO: 36: Sting sgRNA
SEQ ID NO: 37: Sting sgRNA

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H Functional screening identifies Ptpn2 as a suppressor of axon regeneration. (FIG. 1A) Quantification of in-vitro screening on shRNAs of mouse phosphatases. Adult DRG neurons were dissociated and transfected with respective plasmids for 3 days. Neurons were then replated and fixed 24 h after replating. Transfected neurons were visualized by tGFP staining. (FIG. 1B) Sample images of replated neurons from respective shRNA groups with Tuj 1 staining. Scale bar: 400 μm. (FIG. 1C) Quantification of in-vitro screening on inhibitors of mouse phosphatases. Adult DRG neurons were cultured for 2 days and treated by respective inhibitors for another 1 day. *p≤0.05, ANOVA Stat Test followed by Dunnett's test. (FIG. 1D) Sample images of replated neurons from respective treatment groups with Tuj 1 staining. Scale bar: 400 μm. (FIG. 1E) Sections of optic nerves from WT mice at 2 WPI. The vitreous body was injected with either DMSO-containing vehicle or 10 μM PTPN2 inhibitor immediately after optic nerve injury. Axons were labeled by cholera toxin B subunit (CTB). Scale bar: 200 μm. (FIG. 1F) Number of regenerating axons at indicated distances from the lesion site. **p≤0.01, ANOVA Stat Test followed by Tukey HSD test, n=7-9 mice. (FIG. 1G) Sections of optic nerves from WT or Ptpn2 floxed mice at 2 weeks post-injury (WPI). The Ptpn2 conditional knockout (cKO) was induced by intravitreal injection of AAV-Cre. Scale bar: 200 μm. (FIG. 1H) Number of regenerating axons at different distances from the lesion site. **p≤0.01, ANOVA followed by Bonferroni's test, n=6 mice.

FIGS. 2A-2E Ptpn2 deletion effect is boosted by IFNγ and synergizes with Pten/Socs3 codeletion for long-distance axon regeneration. (FIG. 2A) Sections of optic nerves from WT or Ptpn2 cKO mice with IFNγ treatment. The vitreous body was injected with IFNγ immediately after optic nerve injury. Scale bar: 200 μm. (FIG. 2B) Number of regenerating axons at indicated distances distal to the lesion site. **p≤0.01, *p≤0.05, ANOVA followed by Tukey's test, n=5-7 mice. (FIG. 2C) Section of optic nerves from Pten; Socs3 double floxed mice or Pten; Socs3; Ptpn2 triple floxed mice at 2WPI. The vitreous body was injected with AAV-Cre combined with AAV-CNTF. IFNγ or PBS was injected into the vitreous body immediately after optic nerve injury. Asterisks indicate the lesion site. Scale bar: 500 μm. (FIG. 2D) Quantification of regenerating axons at indicated distances from the lesion site. **p≤0.01, ANOVA followed by Bonferroni's test, n=7 mice. Error bars indicate SEM. (FIG. 2E) Quantification of the number of axons grows into the optic chiasm in FIG. 10C. **p≤0.01, Student's t test, n=8-9 mice. Error bars indicate SEM.

FIGS. 3A-3F Neuronal Ptpn2 deletion sustains IFNγ-IFNGR-Stat1 signaling to promote axon regeneration in CNS. (FIG. 3A) Number of regenerating axons at indicated distances distal to the lesion site in FIG. 11D. **p≤0.01, *p≤0.05, ANOVA followed by Tukey's test, n=4-6 mice. Error bars indicate SEM. (FIG. 3B) Quantification of the percentage of p-STAT1+RGCs at indicated time points after injury in FIG. 11F. **p≤0.01, ANOVA followed by Bonferroni's test. n=3-5 mice. (FIG. 3C) Sections of optic nerves from WT, Mtor floxed, Jak1 foxed or Stat3 foxed mice at 2WPI. The vitreous body was injected with AAV-Cre or AAV-sh-Stat1 combined with AAV-sh-Ptpn2. IFNγ was injected into the vitreous body immediately after optic nerve injury. Scale bar: 200 μm. (FIG. 3D) Number of regenerating axons at indicated distances from the lesion site. **p≤0.01, *p≤0.05, ANOVA followed by Tukey's test, n=5-6 mice. (FIG. 3E) Sections of optic nerves from WT or Stat3 foxed mice at 2WPI. The vitreous body was injected with AAV-Cre, AAV-sh-ctrl or AAV-sh-Stat1 combined with AAV-CNTF. Scale bar: 200 μm. (FIG. 3F) Quantification of regenerating axons at indicated distances distal to the lesion site. **p≤0.01, ANOVA followed by Tukey's test, n=4 mice.

FIGS. 4A-4G. cGAS-STING cytosolic DNA sensing pathway mediates axon regeneration induced by IFNγ. (FIG. 4A) Retinal sections from WT or Ptpn2 cKO mice with PBS or IFNγ treatment. The samples were collected 2 days after injury and stained for Tuj 1 (green), and cGAS (red). Scale bar: 50 μm. (FIG. 4B) Quantification of the percentage of cGAS+RGCs in (A). **p≤0.01, ANOVA followed by Dunnett's test. n=3-4 mice. (FIG. 4C) Sections of optic nerves from WT, Cgas KO, Sting KO or Mavs KO mice at 2WPI. The vitreous body was injected with AAV-sh-Ptpn2. 0.1 μg/μL IFNγ was injected into the vitreous body immediately after optic nerve injury. Scale bar: 200 μm. (FIG. 4D) Number of regenerating axons at indicated distances from the lesion site. **p≤0.01, ANOVA followed by Tukey's test, n=4 mice. Error bars indicate SEM. (FIG. 4E) Quantification of regenerating axons in WT, Cgas KO, Sting KO or Mays KO mice. ANOVA followed by Tukey's test, n=3 mice (FIG. 4F) Sections of optic nerves from WT mice at 2WPI. The vitreous body was injected with 25 mM cGAMP immediately after optic nerve injury. Scale bar: 200 (FIG. 4G) Number of regenerating axons at indicated distances from the lesion site. **p≤0.01, ANOVA followed by Tukey's test, n=4-5 mice.

FIGS. 5A-5K Axonal IFNγ is locally translated upon axotomy in PNS but not in CNS. (FIG. 5A) A diagram shows the method of sciatic nerve injury and spinal cord injury. Arrowhead labels the location of the lesion site. (FIG. 5B) Longitude sections of sciatic nerves from WT animals at different time points (intact, 3hpc and 3dpc) after injury, stained with IFNγ (red) and NFH (green) antibodies. Scale bar: 200 μm. Zoom-in images of the 3hpc sciatic nerve section from (B) are shown in B′ box. Scale bar: 50 μm. Asterisks indicate the lesion site. (FIG. 5C) Cross sections of sciatic nerves from WT animal at 3dpc, stained with IFNγ (red), MBP (white) and NFH (green) antibodies. Scale bar: 10 μm. (FIG. 5D) Validation of IFNγ expression in DRG or sciatic nerve lysate by western blot. (FIG. 5E) Quantification of IFNγ expression in (FIG. 5D). n=4 mice. **p≤0.01, ANOVA followed by Tukey's test. (FIG. 5F) Representative images of the longitude section of an intact sciatic nerve. IFNγ mRNA (red) was stained by in-situ hybridization and Tuj 1 protein was stained by the antibody. Arrowheads indicate the axonal IFNγ mRNA. Scale bar: 20 μm. (FIG. 5G) Sections of injured nerve segments after 4-hour DMEM incubation, stained with IFNγ (red) and NFH (green). Anisomycin was added to DMEM to suppress local translation. Asterisks indicate the lesion site. Scale bar: 100 μm. (FIG. 5H) Quantification of the relative intensity of IFNγ in (FIG. 5G). **p≤0.01, Student's t test, n=4 nerves. (FIG. 5I) Cross sections of injured sciatic nerves from WT animals with intrathecal injection of AAV-ctrl or Ifng-shRNA were stained with IFNγ (red), MBP (grey) and NFH (green). Scale bar: 10 μm. (FIG. 5J) Sections of the spinal cord from Thy1-GFP animal before injury or at 3hpc after injury, stained with IFNγ (red), and GFP (green) antibodies. Asterisk labels the location of the lesion site. Scale bar: 400 μm. (FIG. 5K) A diagram shows the method of optic nerve injury. Confocal images are sections of the optic nerve from Thy1-GFP animal before injury or at 3hpc after injury, stained with IFNγ (red), and GFP (green) antibodies. Asterisk labels the location of the lesion site. Scale bar: 50 μm.

FIGS. 6A-6G Axonal IFNγ and its subsequent activation within the nerve are required for the peripheral axon regeneration. (FIG. 6A) Representative sections from sciatic nerves of WT animals with respective virus injection at 3dpc. Regenerating axons were visualized by SCG10 staining. Dotted lines indicate the proximal side of the lesion site. Scale bar: 200 μm. (FIG. 6B) Quantification of sensory axon regeneration in (A). *p≤0.05, Student's t test, n=4 mice. (FIG. 6C) Quantification of percentages of pSTAT3 positive DRG neurons at 3dpc. Sciatic nerve crush was done four weeks after AAV-ctrl or Ifng-shRNA injection. ns, not significant, Student's t test, n=4 mice. (FIG. 6D) Quantification of percentages of pcJun positive DRG neurons at 3dpc. Sciatic nerve crush was done four weeks after AAV-ctrl or Ifng-shRNA injection. ns, not significant, Student's t test, n=4 mice. (FIG. 6E) A diagram shows the method of sciatic nerve injection of ctrl or IFNGR1 neutralizing antibody. (FIG. 6F) qPCR analysis of ISG expression in at 1dpc. Ctrl or IFNGR1 antibody (5 mg/ml) was injected into the sciatic nerve after injury. (FIG. 6G) Quantification of sensory axon regeneration of respective groups in (FIG. 6E) at 3dpc. **p≤0.01, Student's t test, n=4 mice.

FIGS. 7A-7L Axonal IFNγ upregulates cGAS in the non-neuronal cells of the sciatic nerve to promote regeneration. (FIG. 7A) Western blots of IFNγ and cGAS in sciatic nerve lysate at different time points after injury. (FIG. 7B) Quantification of IFNγ expression level in (FIG. 7A). **p≤0.01, *p≤0.05, ANOVA followed by Dunnett's test. (FIG. 7C) Quantification of cGAS expression level in (FIG. 7A). n=3 nerves. **p≤0.01, ANOVA followed by Dunnett's test. (FIG. 7D) Sections of intact or 3dpc sciatic nerves from WT animal, stained with cGAS (red) antibody. Asterisk labels the lesion site. Scale bar: 200 μm. (FIG. 7E) Section of a sciatic nerve from WT animal at 3dpc was stained with Tuj 1 (green) and cGAS (red). Arrowhead labels cGAS+/Tuj 1-cells. Scale bar: 50 μm. (FIG. 7F) Section of a sciatic nerve from WT animal at 3dpc was stained with CD45 (green) and cGAS (red). Arrow labels double positive cells and arrowhead labels cGAS+/CD45-cells. Scale bar: 50 μm. (FIG. 7G) Section of a sciatic nerve from WT animal at 3dpc was stained with S100β (green) and cGAS (red). Arrow labels double-positive cells and arrowhead labels cGAS+/S100β-cells. Scale bar: 50 μm. (FIG. 7H) Western blots of cGAS in sciatic nerve lysate at 1dpc. Ctrl, IFNAR1 or IFNGR1 neutralizing antibodies (5 mg/ml) were injected into the sciatic nerve after injury. (FIG. 7I) Quantification of cGAS expression in (FIG. 7H). n=3 nerves. **p≤0.01, ANOVA followed by Dunnett's test. (FIG. 7J) A diagram shows the method of sciatic nerve injection of DMSO vehicle or RU.521 (1 mM). (FIG. 7K) Shown are representative sections from sciatic nerves of WT animals with DMSO vehicle or RU.521 (1 mM) injection. Regenerating axons were visualized by SCG10 staining. Dotted lines indicate the proximal side of the lesion site. Scale bar: 200 μm. (FIG. 7L) Quantification of sensory axon regeneration in (FIG. 7K). *p≤0.05, Student's t test, n=5 mice.

FIGS. 8A-8G cGAMP promotes peripheral axon regeneration through axonal STING. (FIG. 8A) Representative images of DRG neurons in primary cultures treated with DMSO vehicle, cGAMP (10 μM), DMXAA (10 μM), C-176 (1 μM) or H-151 (1 μM). Scale bar: 400 μm. (FIG. 8B) Quantification of lengths of the longest axon for each DRG neuron in (FIG. 8A). n=3 mice. **p≤0.01, *p≤0.05, ANOVA followed by Dunnett's test. (FIG. 8C) Representative images of DRG neurons from WT, Sting KO or Mavs KO mice in primary cultures treated with DMSO vehicle or cGAMP (10 μM). Scale bar: 400 (FIG. 8D) Quantification of lengths of the longest axon for each DRG neuron in (FIG. 8C). n=3 mice. **p≤0.01, ANOVA followed by Tukey's test. (FIG. 8E) Representative images of embryonic DRG culture in the compartmented chamber. ADU-S100 (10 μM) was added to the axonal or soma chamber after in-vitro axotomy. Scale bar: 400 μm. (FIG. 8F) Quantification of neurite lengths in (FIG. 8E). *p≤0.05, ANOVA followed by Dunnett's test, n=5-6 batches of primary culture. (FIG. 8G) A working model of IFNγ-STAT1 signaling promoting axon regeneration in CNS and PNS. In CNS, IFNγ activates STAT1 in Ptpn2 cKO RGCs. STAT1 then upregulates neuronal cGAS expression. cGAS produces cGAMP and activate STING in neurons. In PNS, axotomy induces IFNγ expression in local axons. And IFNγ activates STAT1-cGAS signaling and cGAMP production in surrounding Schwann cells and blood cells, which promote peripheral axon regeneration.

FIGS. 9A-9K. Neuronal Ptpn2 KO in dorsal root ganglion promotes axon regeneration and enhances interferon response genes after axotomy. (FIG. 9A) Schematic representation of the generation of Ptpn2 floxed mouse by knockout first allele. Tm1a was generated using a gene-trap cassette containing the marker genes lacZ and neomycin. A floxed allele in tm1c was then created by crossing tm1a with the Flp recombinase mouse line. (FIG. 9B) Representative image of brain section of Rosa26-LSL-TMT reporter line. Cortical injection of AAV-Cre resulted in efficient recombination in the TMT reporter line. Scale bar: 1 mm. (FIG. 9C) Western blots of lysates of the cortex of Ptpn2 floxed mice injected with AAV-GFP or AAV-Cre. GAPDH was used as the loading control. (FIG. 9D) Sample images of replated neurons infected by AAV-GFP or AAV-Cre with Tuj 1 staining. Scale bar: 400 μm. (FIG. 9E) Quantification of the length of the longest axon for each DRG neuron in (FIG. 9D). Three mice and 10-20 neurons from each mouse were quantified in each group. **p≤0.01, Student's t test. (FIG. 9F) Sciatic nerve crush was performed two weeks after Tamoxifen injection into the Advillin-CreERT2/Ptpn2 foxed mice (Ptpn2 cKO). Shown are representative sections from sciatic nerves of WT or Ptpn2 cKO animals at 2 days post crush (dpc). Regenerating axons were visualized by SCG10 staining. Dotted lines indicate the proximal side of the lesion site. Scale bar: 200 μm. (FIG. 9G) Quantification of sensory axon regeneration in (FIG. 9F). *p≤0.05, Student's t test, n=4 mice. (FIG. 9H) A whole-mount retina from WT mice and Ptpn2 cKO mice at 14dpc with Tuj 1 staining. Scale bar: 50 μm. (FIG. 9I) Quantification of surviving RGCs from WT and Ptpn2 cKO retina at 14 dpc. ns, not significant. (FIG. 9J) Bar chart showing the GO Enrichment of upregulated pathways in Ptpn2 cKO DRGs after injury. (FIG. 9K) Heatmap showing the expression level of ISGs in WT or Ptpn2 cKO DRGs before or after injury.

FIGS. 10A-10C. Screen exogenous factors to enhance neuronal Ptpn2 KO effect and Pten/Socs3/Ptpn2 triple deletion elicits long-distance regeneration at 4 weeks after optic nerve crush. (FIG. 10A) Quantification of regenerating axons from WT or Ptpn2 cKO mice with PBS or indicated protein injection. Leptin: 2 μg/μL, insulin: 2 μg/μL, EGF: 1 μg/μL. bNGF: 1 μg/μL, IFNγ: 0.1 μg/μL. **p≤0.01, ANOVA followed by Tukey's test, n=5-8 mice. Error bars indicate SEM. (FIG. 10B) Quantification of surviving RGCs at 2 WPI. ns, not significant. ANOVA followed by Dunnett's test. n=5. Error bars indicate SEM. (FIG. 10C) Section of optic nerves from Pten; Socs3 double floxed mice or Pten; Socs3; Ptpn2 triple floxed mice at 4WPI. The vitreous body was injected with AAV-Cre combined with AAV-CNTF. IFNγ or PBS was injected into the vitreous body immediately after optic nerve injury. Asterisks indicate the lesion site. Scale bar: 500 μm. Zoomed-in images of optic chiasm are shown in the bottom panel. Panel C′ shows zoomed-in images of optic chiasm from (FIG. 10C). Scale bar: 500 μm.

FIGS. 11A-11G RNA-Seq analysis of regenerating RGCs induced by Ptpn2 KO plus IFNγ and IFNγ-Stat1 induced regeneration is independent of Stat3 signaling. (FIG. 11A) Time course of virus injection, optic nerve crush and retrograde labelling for RNA-seq experiment. A diagram shows the retrograde labelling method for RGCs. (FIG. 11B) Bar chart showing the GO Enrichment of upregulated pathways in Ptpn2 cKO RGCs. (FIG. 11C) Volcano plot of RNA seq data of WT and Ptpn2 cKO RGCs after IFN-γ injection and injury. The log2 (FoldChange) of the difference was plotted on the x-axis and the −log 10 (pValue) was plotted on the y-axis. Red dots represent ISGs. (FIG. 11D) Sections of optic nerves from Ptpn2 foxed mice at 2WPI. The vitreous body was injected with AAV-Cre combined with respective AAV-shRNA. IFNγ was injected into the vitreous body immediately after optic nerve injury. Scale bar: 200 μm. (FIG. 11E) Sections of retinas from WT or Ptpn2 cKO mice with IFNγ treatment. The samples were collected at indicated time points after injury and stained for Tuj 1 (green), and p-STAT1 (red). Scale bar: 50 μm. (FIG. 11F) Quantification of the percentage of p-STAT3+RGCs in respective groups at 1 dpc. **p≤0.01, ANOVA followed by Dunnett's test. n=4 mice. Error bars indicate SEM. (FIG. 11G) The eyes of WT or ifngr1 foxed mice were injected with AAV-Cre combined with AAV-CNTF. The optic nerve regeneration was assessed at 2WPI. Quantification of regenerating axons at indicated distances distal to the lesion site. ANOVA followed by Bonferroni's test, n=4 mice. Error bars indicate SEM.

FIGS. 12A-12J. Neuronal Ptpn2 KO plus IFNγ activate cGAS in injured RGCs and neuronal Sting mediates the regeneration. (FIG. 12A) AAV-GFP or Cre was injected into the eyes of Ptpn2 flox mice. IFNγ injection and optic nerve crush were done two weeks after virus injection. Shown are representative western blots of retina lysates. GAPDH was used as the loading control. (FIG. 12B) Quantification of the band intensity in (FIG. 12A). 3 retinas were used in each group. **p≤0.01, ANOVA followed by Tukey's test. (FIG. 12C) Representative western blots of retinas from WT, Sting KO or Cgas KO mice. GAPDH was used as the loading control. (FIG. 12D) Quantification of Tuj 1+ surviving RGCs at 2 WPI. ANOVA followed by Dunnett's test, n=3-4 mice. Error bars indicate SEM. (FIG. 12E) Sections of optic nerves from WT mice at 2 WPI. The vitreous body was injected with either PBS or ADU-S100 with indicated concentration immediately after optic nerve injury. Scale bar: 200 μm. (FIG. 12F) Quantification of regenerating axons from WT mice with ADU-S100 treatment. **p≤0.01, *p≤0.05, ANOVA followed by Tukey's test, n=4-5 mice. Error bars indicate SEM. (FIG. 12G) Cas9 was stably transduced into N2a cells. Control or Sting sgRNA were transfected to N2a and then cells were lysed 72 hrs after transfection. Western blots show the STING expression. GAPDH was used as the loading control. (FIG. 12H) Whole-mount retina from Vglut2-Cre; Rosa26-LSL-Cas9-EGFP mouse was stained for Tuj 1 (red), and EGFP (green). Showing the expression of Cas9 in the RGCs. Scale bar: 50 μm. (FIG. 12I) Section of optic nerves from Vglut2-Cre; Rosa26-LSL-Cas9-EGFP mice at 2 WPI. The vitreous body was injected with AAV-ctrl-sgRNA or AAV-Sting-sgRNA. 25 Mm cGAMP was injected into the vitreous body immediately after optic nerve injury. Scale bar: 200 μm. (FIG. 12J) Quantification of regenerating axons at indicated distances from the lesion site. **p≤0.01, ANOVA followed by Bonferroni's test, n=3 mice. Error bars indicate SEM.

FIGS. 13A-13D. Elevation of IFNγ in injured peripheral axons does not depend on axonal transport. (FIG. 13A) Representative images of sciatic nerve sections of the double ligation model. Double ligation was performed on sciatic nerves and the nerves were fixed three hours after surgery. Sections were stained with IFNγ (red) and NFH (green) antibodies. Scale bar: 200 μm. (FIG. 13B) Representative images of DRG neurite in culture. IFNγ mRNA (red) was stained by in-situ hybridization and Tuj 1 protein was stained by the antibody. Arrowheads indicate the axonal IFNγ mRNA. Scale bar: 20 μm. (FIG. 13C) Quantification of IFNγ positive axons in FIG. 5I. **p≤0.01, Student's t test, n=4 mice. (FIG. 13D) Sections of spinal cord or optic nerve from Thy1-GFP animal at 3dpc after injury, stained with IFNγ (red). Asterisks labels the location of lesion site. Scale bar for spinal cord: 400 Scale bar for optic nerve: 10 μm.

FIGS. 14A-14H. Neuronal IFNGR1 and cGAS are not required for the axon regeneration of dorsal root ganglion neurons. (FIG. 14A) Expression level of respective ISGs in DRGs three days after sham surgery, DCA or SNA. *p≤0.05, **p≤0.01, ns, not significant, Student's t test, n=4 mice. (FIG. 14B) Diagram shows the intrathecal injection of AAVs leading to the expression of GFP or Cre in DRG neurons. (FIG. 14C) Section of DRG from Rosa26-LSL-TMT mouse injected with AAV-Cre was stained with Tuj 1 (green). Scale bar: 100 μm. (FIG. 14D) qPCR analysis of ISG expression in DRGs at 1dpc. AAV-GFP or Cre was injected into the sciatic nerve four weeks before injury. **p≤0.01, *p≤0.05, ANOVA followed by Tukey's test, n=3 mice. (FIG. 14E) AAV-GFP or Cre were injected intrathecally to Ifngr1-flox animals. Sciatic nerve crush was performed four weeks after injection. Shown are representative sections from sciatic nerves with respective virus injections at 3dpc. Regenerating axons were visualized by SCG10 staining. Dotted lines indicate the proximal side of the lesion site. Scale bar: 200 (FIG. 14F) Quantification of sensory axon regeneration of respective groups in (FIG. 14E). ns, not significant, Student's t test, n=4 mice. Error bars indicate SEM. (FIG. 14G) Representative images of DRG neurons in primary cultures treated with DMSO vehicle, or RU.521 (10 μM). DRG neurites were visualized by Tuj 1 staining. Scale bar: 400 (FIG. 14H) Quantification of lengths of the longest axon for each DRG neuron in (FIG. 14G). 10-20 cells from each mouse and 3 mice were quantified in each group. ns, not significant, Student's t test.

FIGS. 15A-15B. IFNγ receptor antibody infusion suppresses cGAS activation in the injured sciatic nerve. (FIG. 15A) Section of sciatic nerve injected with ctrl or IFNGR1 neutralizing antibody (5 mg/ml) at 1 dpc was stained with DAPI (blue) and cGAS (red). Dotted lines indicate the proximal side of the lesion site. Scale bar: 200 (FIG. 15B) Quantification of percentages of cGAS positive cells in injured nerves in (FIG. 15A). **p≤0.01, *p≤0.05, ANOVA followed by Bonferroni's test, n=4 mice. Error bars indicate SEM.

FIGS. 16A-16B. Exogenous cGAMP promotes axon elongation through importation. (FIG. 16A) Representative images of DRG neurons in primary cultures treated with DMSO vehicle, cGAMP (10 μM), DCPIB (100 nM) or CBX(10 μM). DRG neurites were visualized by Tuj 1 staining. Scale bar: 400 μm. (FIG. 16B) Quantification of lengths of the longest axon for each DRG neuron in (FIG. 16A). 10-20 cells from each mouse and 3 mice were quantified in each group. **p≤0.01, ANOVA followed by Tukey's test. Error bars indicate SEM.

### DETAILED DESCRIPTION

The subject invention pertains to compositions and methods of altering an antiviral mechanism in neurons for axon regeneration in the central nervous system (CNS) and/or the peripheral nervous system (PNS). In certain embodiments, the subject invention pertains to a novel methods to utilize the cGAS-STING pathway and STAT1 signaling in order to regenerate axons in the CNS. Advantageously, the subject methods can also treat a subject suffering from a central nervous system (CNS) injury, which can be caused by, for example, a spinal cord injury, traumatic brain injury, stroke, glaucoma, violent blow or jolt to the head or body, or any combination thereof.

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, or improving a condition, disease, or disorder in a subject. In other words, when administered to a subject, the amount is “therapeutically effective.” The actual amount will vary depending on a number of factors including, but not limited to, the particular condition, disease, or disorder being treated, prevented, or improved; the severity of the condition; the weight, height, age, and health of the patient; and the route of administration.

As used herein, the term “treatment” refers to eradicating; reducing; ameliorating; abatement; remission; diminishing of symptoms or delaying the onset of symptoms; slowing in the rate of degeneration or decline; making the final point of degeneration less debilitating; and/or improving a subject's physical or mental well-being or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, 50, 60, 70, 80, 90, 100 or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, 2 months, 3 months, 6 months, 9 months, 1 year, 1.5 years, 2 years, 2.5 years, 5 years or more than 10 years. The frequency and duration of administration of multiple doses of the compositions is such as treat a CNS or PNS injury. In certain embodiments, doses are administered daily, every other day, twice per week, weekly, biweekly, monthly, quarterly, twice per year, yearly, or biyearly. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of testing for a CNS or PNS injury. In certain embodiments, a test for a PNS injury is, for example, a sciatic nerve crush injury. In certain embodiments, a test for a CNS injury is, for example, an optic nerve crush injury. In some embodiments of the invention, the method comprises administration of the compounds at several time per day, including but not limiting to 2 times per day, 3 times per day, and 4 times per day.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, “vector” refers to a DNA molecule such as a plasmid for introducing a nucleotide construct, for example, a DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide a selectable characteristic, such as tetracycline resistance, hygromycin resistance or ampicillin resistance.

As used in herein, the terms “identical” or “percent identity”, in the context of describing two or more polynucleotide sequences, refer to two or more sequences or subsequences that are the same or have a specified percentage of nucleotides that are the same over the compared region. For example, a homologous nucleotide sequence used in the method of this invention has at least 80% sequence identity, preferably 85%, 90%, 91%, 92%, 93, 94%, 95%, 96%, 97%, 98%, 99%, or 100% identity, to a reference sequence, when compared and aligned for maximum correspondence over a comparison window, or over a designated region as measured using a comparison algorithms or by manual alignment and visual inspection. With regard to polynucleotide sequences, this definition also refers to the complement of a test sequence.

An endogenous nucleic acid is a nucleic acid that is naturally present in a cell. For example, a nucleic acid present in the genomic DNA of a cell is an endogenous nucleic acid.

An exogenous nucleic acid is any nucleic acid that is not naturally present in a cell. For example, a nucleic acid vector introduced into a cell constitutes an exogenous nucleic acid. Other examples of an exogenous nucleic acid include the vectors comprising a heterologous promoter linked to an endogenous nucleic acid, e.g., a nucleic acid encoding a kinase.

The subject invention provides for the use of “homologous nucleic acid sequences” or “homologs of nucleic acid sequences”. Homologs of nucleic acid sequences will be understood to mean any nucleotide sequence obtained by mutagenesis according to techniques well known to persons skilled in the art, and exhibiting modifications in relation to the parent sequences. For example, mutations in the regulatory and/or promoter sequences for the expression of a polypeptide that result in a modification of the level of expression of a polypeptide according to the invention provide for a “homolog of a nucleotide sequence”. Likewise, substitutions, deletions, or additions of nucleic acid to the polynucleotides of the invention provide for “homologs” of nucleotide sequences. In various embodiments, “homologs” of nucleic acid sequences have substantially the same biological activity as the corresponding reference gene, i.e., a gene homologous to a native gene would encode for a protein having the same biological activity as the corresponding protein encoded by the naturally occurring gene. Typically, a homolog of a gene shares a sequence identity with the gene of at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. These percentages are purely statistical and differences between two nucleic acid sequences can be distributed randomly and over the entire sequence length.

The phrase “a transformed cell” as used herein refers to a cell in which the cells are transformed with a DNA vector or plasmid disclosed herein.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

As used herein, “subject”, “host” or “organism” refers to any member of the phylum Chordata, more preferably any member of the subphylum vertebrata, or most preferably, any member of the class Mammalia, including, without limitation, humans and other primates, including non-human primates such as rhesus macaques, chimpanzees and other monkey and ape species; livestock, such as cattle, sheep, pigs, goats and horses; domestic mammals, such as dogs and cats; laboratory animals, including rabbits, mice, rats and guinea pigs; birds, including domestic, wild, and game birds, such as chickens, turkeys, ducks, and geese. The term does not denote a particular age or gender. Thus, adult, young, and new-born individuals are intended to be covered as well as male and female subjects. In some embodiments, a host cell is derived from a subject (e.g., tissue specific cells or stem cells). In some embodiments, the subject is a non-human subject.

### Compounds and Methods for Treating CNS or PNS Injury

Provided are compounds and compositions for treating CNS and/or PNS injury. Advantageously, the methods of the invention comprise administering to a subject an effective amount of IFNγ and a Protein Tyrosine Phosphatase Non-Receptor Type 2 (PTPN2) inhibitor, a STING agonist, or a combination thereof to the subject. In certain embodiments, the methods of the invention comprise inhibiting the expression of PTPN2 and administering IFNγ; administering a STING agonist; or a combination thereof.

In certain embodiments, the PTPN2 is suppressed using a small hairpin RNA (shRNA) targeting Ptpn2. In certain embodiments, PTPN2 can be inhibited with a PTPN2 inhibitor including, for example, compound 8 (Formula (I)), compound-182 (Formula (II)), ABBV-CLS-484 (Formula (III)), or any combination thereof.

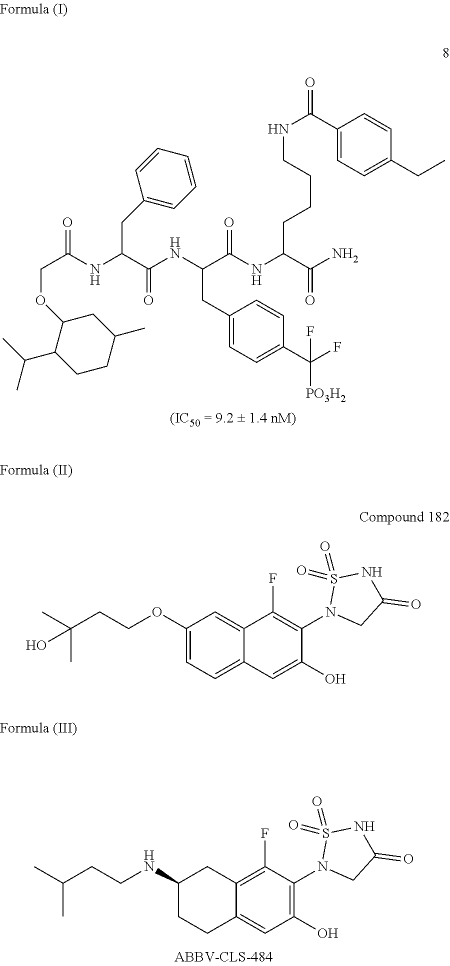

In certain embodiments, the concentration of the PTPN2 inhibitor is about 5 μM to about 10 μM in a composition relative to a composition that has only the PTPN2 inhibitor.

In certain embodiments, the STING agonist is 2′,3′-cGAMP, ADU-S100, 3′,3′-cGAMP, 5,6-Dimethylxanthenone-4-acetic acid (DMXAA), Cridanimod (10-carboxymethyl-9-acridanone; CMA), 4-(5,6-dimethoxy-1-benzothiophen-2-yl)-4-oxobutanoic acid (MSA 2), or any combination thereof. In certain embodiments, the concentration of the STING agonist is about 1 mM to about 25 mM in a composition relative to a composition that has only the STING agonist.

The skilled artisan will understand that the dosage of the compositions of the instant invention varies, depending upon, for example, the route of administration, other drugs being administered, and the age, condition, gender and seriousness of the CNS and/or PNS injury in the subject. An effective dose of a PTPN2 inhibitor or composition thereof, a STING agonist or a composition thereof, or a composition thereof of the invention generally ranges between about 0.001 μg/kg of body weight and 100 mg/kg of body weight. Examples of such dosage ranges include, but are not limited to, about 0.01 μg/kg to about 1 mg/kg; about 0.1 μg/kg to about 10 μg/kg; about 0.5 μg/kg to about 7.5 μg/kg; about 0.75 μg/kg to about 5 μg/kg; or about 1 μg/kg.

In some embodiments, the therapeutically effective amount of a composition of the subject invention can be administered through intracranial or intravitreous injection or by intravenous or subcutaneous administration or by sustained release systems such as semipermeable matrices of solid hydrophobic polymers containing the compounds of the invention. Administration may be also by way of other carriers or vehicles such as patches, micelles, liposomes, vesicles, implants (e.g. microimplants), synthetic polymers, microspheres, nanoparticles, and the like. In certain embodiments, the coordination compound compositions may be administered using a nanoparticle. In some embodiments, at least one compositions of the instant invention may be formulated for parenteral administration e.g., by injection, for example, bolus injection, intravenous administration, or continuous infusion. In addition, the compositions may be presented in unit dose form in ampoules, pre-filled syringes, and small volume infusion or in multi-dose containers with or without an added preservative. The compositions may be in forms of suspensions, solutions, or emulsions in oily or aqueous vehicles. The composition may further contain formulation agents such as suspending, stabilizing and/or dispersing agents. In further embodiments, the active ingredients of the compositions according to the instant invention may be in powder form, obtained by aseptic isolation of sterile solid or by lyophilization from solution for constitution with a suitable vehicle, e.g., sterile, pyrogen-free water, before use.

The subject compositions can further comprise one or more pharmaceutically acceptable carriers, and/or excipients, and can be formulated into preparations, for example, semi-solid or liquid forms, such as solutions or injections.

The term “pharmaceutically acceptable” as used herein means compatible with the other ingredients of a pharmaceutical composition and not deleterious to the recipient thereof.

Carriers and/or excipients according to the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

### Methods of Inhibiting PTPN2

The present disclosure relates to treating various types of CNS and/or PNS injuries by promoting axon regeneration in a subject, by inhibiting the expression of Ptpn2, which encodes the PTPN2 protein.

In certain embodiments, the administration of IFNγ, and the inhibition of the expression of Ptpn2 can be used for treating CNS and/or PNS injury.

Methods for treating a CNS and/or PNS injury can be performed in any subject, such as a mammal, including humans. In certain embodiments, the present invention provides methods for inhibiting Ptpn2 by mutating the nucleotide sequence encoding Ptpn2 with CRISPR/Cas9 technology. In preferred embodiments, amino acid residues and/or nucleotide sequences of Ptpn2 are mutated. In certain embodiments, the CRISPR/Cas9 system induces frameshift mutations to generate gene mutations, and the number of mutated nucleotides is determined by the DNA double-stranded break site, which is in turn determined by the single guide RNA (sgRNA) sequence. In certain embodiments, an sgRNA targeting Ptpn2 can be used with CRISPR/Cas9. In certain embodiments, the sgRNA sequence is GAACCATCGAGCGGGAGTTCG (SEQ ID NO: 19) or a sequence having 90% identity thereof (i.e., a homolog) or GCCATGTCGGCAACCATCGAG (SEQ ID NO: 20) or a sequence having 90% identity thereof (i.e., a homolog).

The methods can further encompass altering the expression of Ptpn2 so as to achieve down-regulation or inhibition of one or multiple target amino acids or to achieve inhibition of the PTPN2. In certain embodiments, a shRNA can be used to alter the expression of Ptpn2 so as to achieve down-regulation or inhibition of one or multiple target amino acids or to achieve inhibition of the PTPN2. In certain embodiments, the shRNA sequence is GACAGAGAAATGGTGTTTAA SEQ ID NO: 21) or a sequence having 90% identity thereof (i.e., a homolog).

In certain embodiments, transcriptional suppression of a target gene is mediated by a gene suppression agent exhibiting substantial sequence identity to a DNA sequence of a target nucleotide sequence or the complement thereof, including promoter sequences of Ptpn2. Inhibition of a target gene or amino acid of the present invention is sequence-specific and can comprise insertions, deletions, and single point mutations relative to the target sequence.

### MATERIALS AND METHODS

### Animals

This experiment utilized different mice to help prove our hypothesis. Wild-type (WT, C57BL/6J, Charles River) mice (7-8 weeks old) of both genders were used. Ptpn2-flox mice were generated from Ptpn2^{tm1a(EUCOMM)Wtsi}. Ptpn2^{tm1a(EUCOMM)Wtsi} are heterozygous mice carrying tm1a alleles generated by the European Conditional Mouse Mutagenesis Program (EUCOMM)/the International Mouse Phenotyping Consortium (IMPC) and they were crossed with Flp mice (gifts from Dr. Jun Xia at The Hong Kong University of Science and Technology) to generate the tm1c conditional ready alleles. Homozygous tm1c mice were used as Ptpn2-floxed mice in our experiments. Pten/Socs3 double-floxed mice were gifts from Dr. Zhigang He (Boston Children's Hospital (Boston, MA)). Jak1-floxed and Stat3-floxed mice were gifts from Dr. Zhenguo Wu (Hong Kong University of Science and Technology (Clear Water Bay, Hong Kong)). Thy1-GFP mice were gifts from Dr. Nancy Ip (Hong Kong University of Science and Technology (Clear Water Bay, Hong Kong)). Mtor-floxed, Ifngr1-floxed, Avl-CreER, Vglut2-Cre, Sting KO, Cgas KO, Mays KO, Rosa26-LSL-TMT and Rosa26-LSL-Cas9 mice were purchased from Jackson Laboratory (Bar Harbor, ME). Avl-CreER mice were crossed with Ptpn2-flox mice to produce the conditional knockout mice in DRG neurons. Pten/Socs3 double-floxed mice were crossed with Ptpn2-floxed mice to generate triple-floxed mice. Vglut2-Cre mice were crossed with LSL-Cas9 mice to induce Cas9 expression specifically in excitatory neurons. Genotypes were confirmed by PCR according to protocols from Jackson Laboratory. Both male and female mice were used for experiments. Experiments were performed in accordance with the guidelines of the Laboratory Animal Facility at the Hong Kong University of Science and Technology.

### Primary Neuronal Culture

In some embodiments, in-vitro screening models of axon regeneration are based on DRG electroporation and replating culture. Adult DRGs were cultured and replated according to the protocol described previously (Weng et al., 2018). In certain embodiments, adult mice were euthanized, and L4-L6 DRGs were dissected from both sides. DRGs were dissociated in 0.5% collagenase for 1.5 hours. Then the medium containing collagenase was replaced by Neurobasal A, and DRGs were gently pipetted 20 times for dissociation. For electroporation, dissociated DRG neurons were transfected by Mouse Neuron Nucleofector Kit in Lonza Nucleofector system using program G103. 6 DRGs and 5 μg plasmid were used for each reaction. Neurobasal-A with 10% B27 supplement was used as the culture medium for DRG neurons.

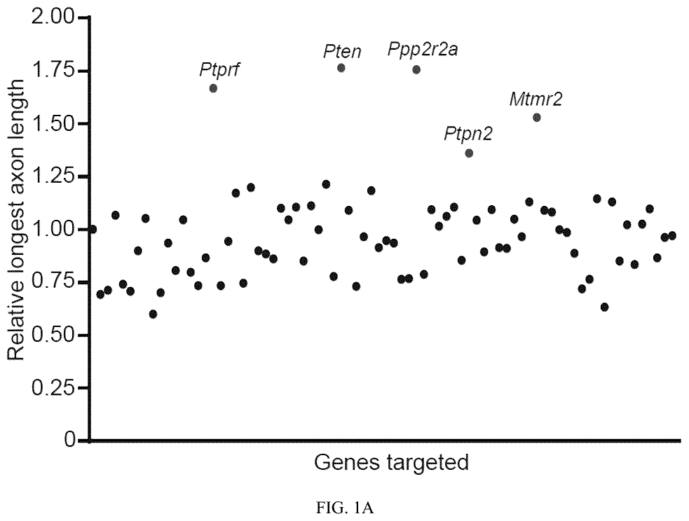

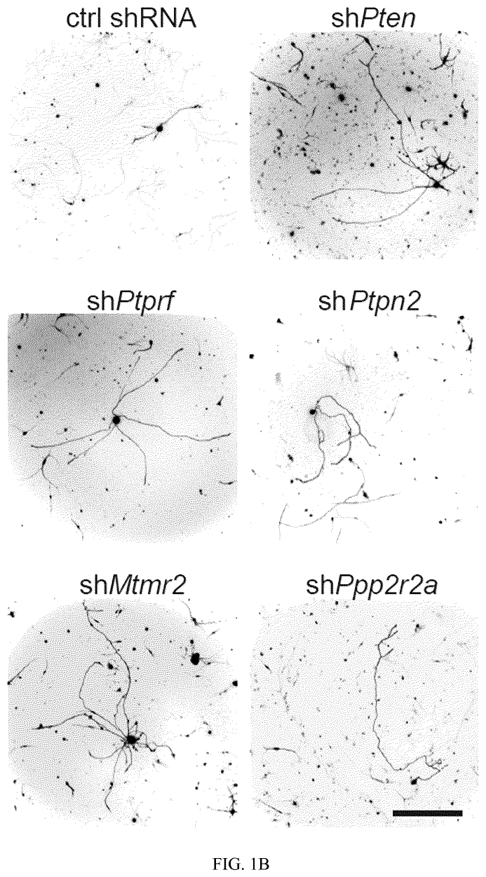

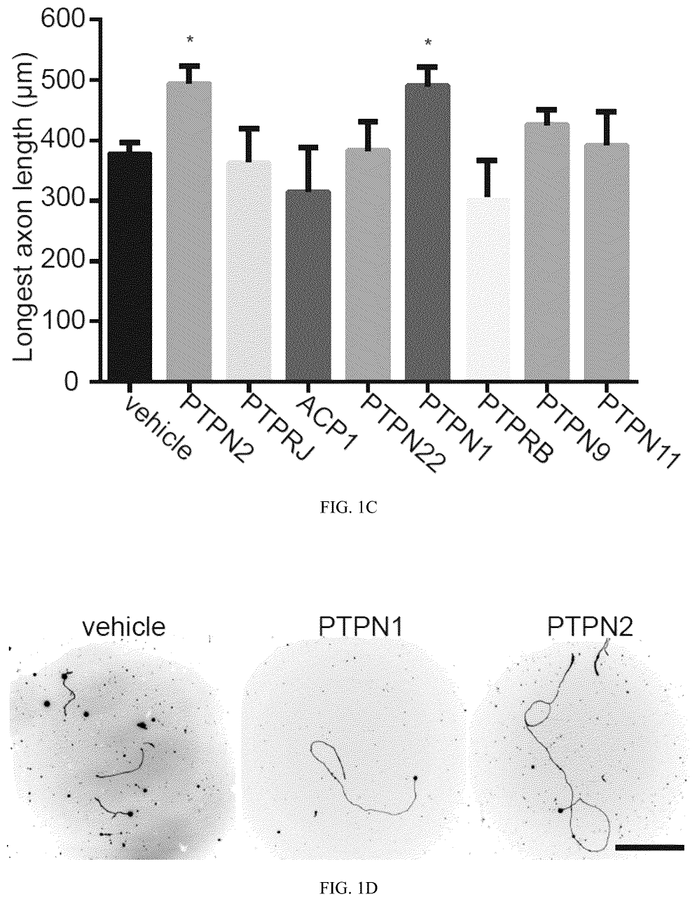

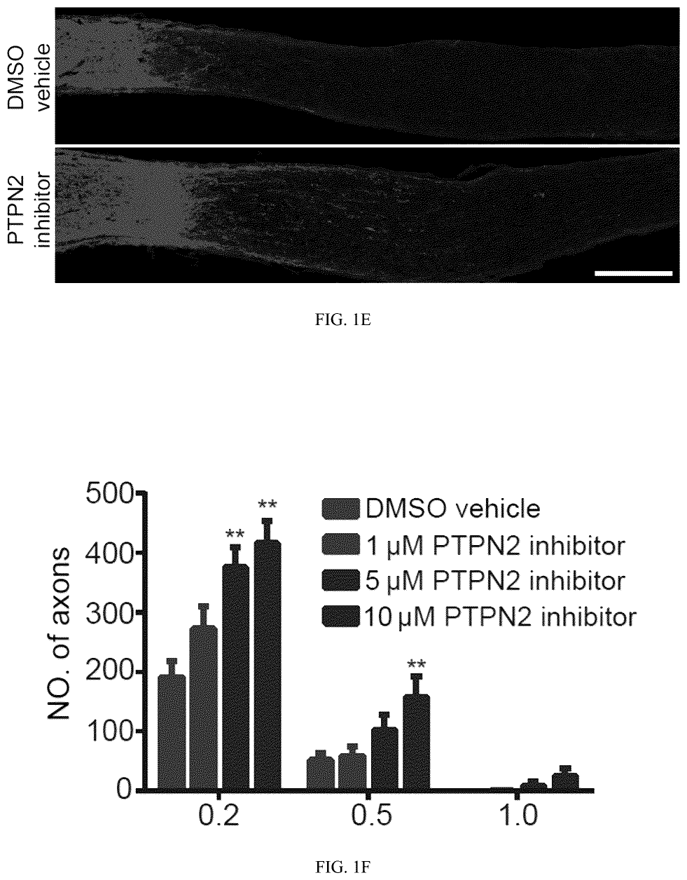

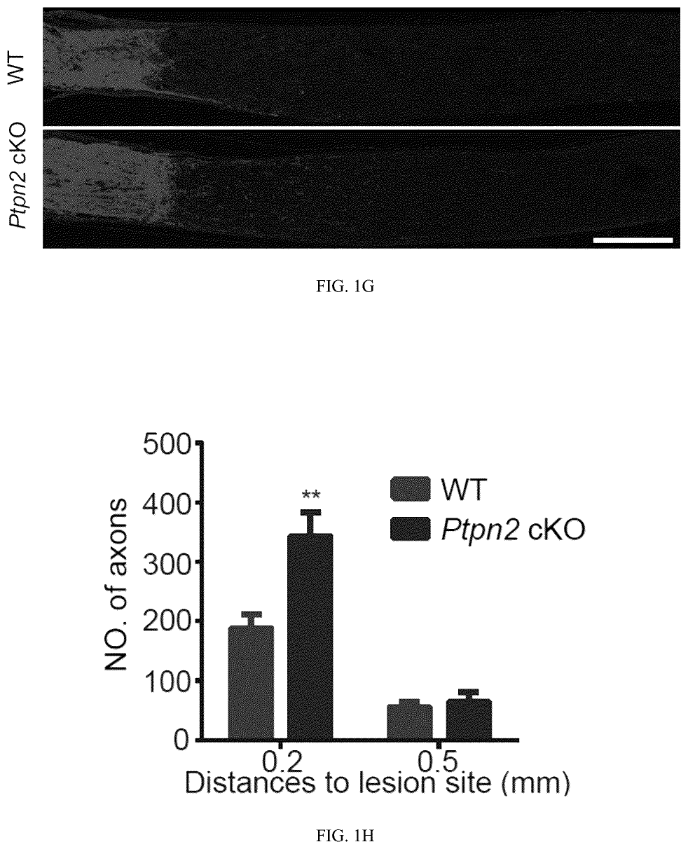

The culture was replated three days after electroporation. The DRG neurons in the primary culture were gently flushed by 20-30 pipetting to be resuspended in the culture medium. Then, the cells were seeded in a new plate. After 24 hours, cells were fixed by PFA and then stained by Tuj 1 and TurboGFP antibodies. The lengths of the longest axons in each DRG neuron were quantified by the NeuronJ plugin in ImageJ. To test the effect of phosphatase inhibitors in the replating culture, respective inhibitors (100 nM) were added to culture medium two days before replating and right after replating. In certain embodiments, the inhibitors are PTPN1, PTPN2, PTPN9, PTPN11, PTPN22, PTPRB, ACP1, and PTPRJ (FIG. 1C).

To culture embryonic DRG in the compartmented chamber, embryonic DRGs were dissected from E13.5 pregnant C57BL/6 mouse embryos in Leibovitz's L-15 medium on ice. Tissues were dissociated in TrypLE for 15 minutes at 37° C. Dissociated neurons were plated in the microfluidic chamber on cleaned glass coverslips pre-coated with poly-D-lysine and laminin in the density of 100000 cells per chamber with 5 μL culture medium supplemented with GlutaMAX (2 mM), B27 (20 ml/l), penicillin-Streptomycin, NGF (50 ng/μL) and 5-fluoro-2′-deoxyuridine (10 μM) into the somal compartment. After one hour, 200 μL medium was added into both the somal and axonal compartment. To perform in-vitro axotomy, axons in the axonal chamber were removed by a glass pipette connected to an aspirator. Then, a 150 μL medium was quickly added into the empty compartment. After 2 days in the vacuum, cells were fixed by 4% PFA and then stained by Tuj 1 antibody. The lengths and/or number of longest axons in each chamber were quantified by ImageJ.

### Neuro2A Cell Line

To test the knockout efficiency of Sting sgRNA, Neuro2A cells stably expressing SpCas9 were cultured in a humidified 5% CO₂ atmosphere at 37° C. using Dulbecco Modified Eagle Medium (DMEM) and supplemented with fetal bovine serum. Cells in 12-well plates were then transfected with 1 μg control or Sting sgRNA plasmid by Lipofectamine 3000 at the confluency between 70% to 80%. The transfection was done according to the manufacturer's protocol. Forty-eight hours after transfection, cell sorting was performed with the BD Bioscience (Franklin Lakes, NJ) FACSAria III instrument to isolate mCherry-positive cells. The harvested cells were lysed for the western blot experiment.

### PTPN2 Inhibitor

The small molecule inhibitor of PTPN2 (compound 8; Formula (I)) was synthesized as described previously (Zhang, S., Chen, L., Luo, Y., Gunawan, A., Lawrence, D. S., and Zhang, Z. Y. (2009). Acquisition of a potent and selective TC-PTP inhibitor via a stepwise fluorophore-tagged combinatorial synthesis and screening strategy. J. Am. Chem. Soc. 131, 13072-13079, which is hereby incorporated by reference in its entirety).

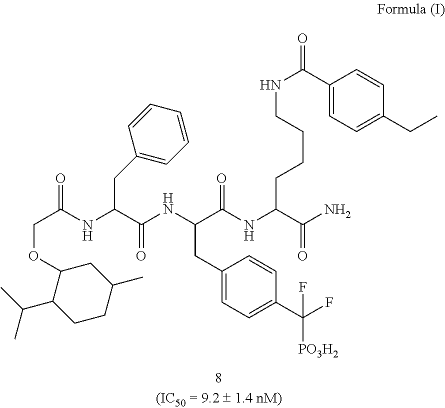

The small molecule inhibitors of PTPN2 (compound-182 (Formula (II) and ABBV-CLS-484 (Formula (III)) were described previously (Liang, S., Tran, E., Du, X. et al. A small molecule inhibitor of PTP1B and PTPN2 enhances T cell anti-tumor immunity. Nat Commun 14, 4524 (2023), which is hereby incorporated by reference in its entirety).

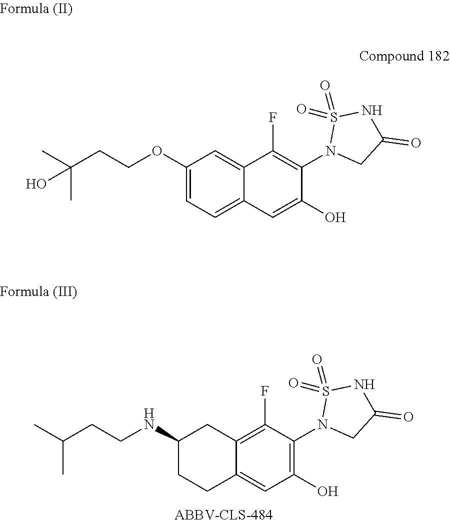 [SMILES: CC(C)(O)CCOc1ccc2cc(O)c(N3CC(=O)NS3(=O)=O)c(F)c2c1 ; CC(C)CCN[C@@H]1CCc2cc(O)c(N3CC(=O)NS3(=O)=O)c(F)c2C1]

### AAV Construct and Packaging

AAV serotype 2/1 was used for CNTF overexpression in the retina. AAV serotype 2/2 was used for shRNA or sgRNA expression in RGCs (Tables 1 and 2). AAV serotype 2/9 was used for shRNA expression in DRGs. The virus titer was measured by qPCR. The virus titer was 10¹³ GC/mL for all the experiments.

TABLE 1
Sequence for shRNA

| | |
|---|---|
| Control shRNA | GACCATCAATATGACTAGA |
| | (SEQ ID NO: 22) |
| Ifnar 1 shRNA | TGTGACACCTTGCTTGTTTAT |
| | (SEQ ID NO: 23) |
| Ifnar 2 shRNA | CCAGATGAACCTTGCACTATA |
| | (SEQ ID NO: 24) |
| Ifngr 1 shRNA | AGCTCTCCGTCCTCGTATTTC |
| | (SEQ ID NO: 25) |
| Ifngr 2 shRNA | GGGACTCCGTGTCAATTATTT |
| | (SEQ ID NO: 26) |
| Stat1 shRNA | CGCCTTTGGGAAGTATTATT |
| | (SEQ ID NO: 27) |
| Pten shRNA | GACTTAGACTTGACCTATATT |
| | (SEQ ID NO: 28) |
| Ptpn2 shRNA | GACAGAGAAATGGTGTTTAA |
| | (SEQ ID NO: 29) |
| Ifng shRNA | GAGCCAGATTATCTCTTTCTA |
| | (SEQ ID NO: 30) |
| Tubb6 shRNA | CCATTCATAATAAAATGCTAA |
| | (SEQ ID NO: 31) |
| Camk1 shRNA | CGGAAGACATTAGGGATATTT |
| | (SEQ ID NO: 32) |
| Apod shRNA | TGGAACCATGAACCAAGTAAA |
| | (SEQ ID NO: 33) |
| Fst shRNA | CCCAACTGCATCCCTTGTAAA |
| | (SEQ ID NO: 34) |

TABLE 2
Sequence for sgRNA

| | |
|---|---|
| Control sgRNA | GCGTCGTGACTGGGAAAACCC (SEQ ID |
| | NO: 35) |
| Sting sgRNA | ACCTGCATCCAGCCATCCCA (SEQ ID |
| | NO: 36) |
| | CACCTAGCCTCGCACGAACT (SEQ ID |
| | NO: 37) |

### Optic Nerve Injury

In certain embodiments, for surgeries, mice were anesthetized with a mixture of ketamine (80 mg/kg) and xylazine (10 mg/kg). Meloxicam (1 mg/kg) was treated as an analgesic after surgical operation. The procedures for intravitreous injection and optic nerve injury were conducted as previously described (Park et al., 2008). In brief, the edge of the eyelid was clamped with an artery clamp to expose the conjunctiva. 2 μL of virus, PTPN2 inhibitor (1 mM in DMSO as stock solution, 1-10 μM diluted in PBS as injection solution) or protein solution (Leptin: 2 μg/μL, insulin: 2 μg/μL, EGF: 1 μg/μL, bNGF: 1 μg/μL, IFNγ: 0.1 μg/μL, diluted in PBS) was injected into the vitreous body with a Hamilton micro-syringe. Eye ointment was applied to the cornea after the operation.

Intraorbital optic nerve injury was performed at 2 weeks to 4 weeks after the virus was injected or right after PTPN2 inhibitor or protein solution injection. The optic nerve was exposed through an incision on the conjunctiva and crushed by forceps (Dumont #5; Fine Science Tools) for 2 s at 1-2 mm distal to the optic disk. To label the regenerated axons, 2 μL CTB-FITC (1 μg/μL) was injected into the vitreous body two days before the mice were terminated.

### Cortical Injection of AAV

AAV-Cre was injected to neonatal Rosa26-LSL-TMT or Ptpn2-flox mice to induce TMT expression or Ptpn2 conditional knockout. Neonatal mice were first cryoanesthetized and 2 μL virus was injected to sensorimotor cortex by injector attached with a glass pipette. Mice were then placed on a warm pad and then returned to their cages when they were fully awake. Mice were euthanized two weeks after injection to check the TMT or Ptpn2 expression.

### Spinal Cord Injury

The procedure of T8 spinal cord complete crush has been described in previous art (Liu et al., 2010). In certain embodiments, an incision can be made on the muscle over the thoracic vertebrae. Then, a laminectomy was performed to expose the dorsal part of the T8 spinal cord. The spinal cord crush was done by Dumont #5 forceps. After surgery, the wounded muscle and skin were sutured. The mice were euthanized three hours or three days after surgery.

### Sciatic Nerve Injury

To induce Ptpn2 cKO in Avl-CreER; Ptpn2-flox mice, tamoxifen (20 mg/ml in corn oil) was administered to mice by oral gavage at 100 mg/kg for three consecutive days. Sciatic nerve crush was performed two weeks after tamoxifen administration.

To knock down Ifng in DRG neurons, the AAV9-ctrl or Ifng-shRNA was delivered by intrathecal injection four weeks before sciatic nerve injury. In certain embodiments, an incision was made on the muscle at the middle thigh. Then, the exposed nerve was crushed by Dumont (Jura, Switzerland) #2 forceps for 20 seconds. For nerve injection of antibodies or compounds, 2 μL ctrl, IFNGR1 or IFNAR1 antibody (5 mg/mL), RU.521 (1 μM), or DMSO vehicle were injected to sciatic nerve right after nerve crush. The mice were euthanized three days after surgery in order to assess axon regeneration.

For double-ligation injury model, sciatic nerves were exposed and ligated by 6-0 surgical sutures for about 3 hours. The two ligation sites were placed 1 mm apart from each other. Mice were euthanized and perfused by 4% PFA after ligation.

For the ex-vivo model of sciatic nerve injury, the nerves were crushed and then cut into about 3 mm to about 5 mm segments. The injured nerve segments were incubated in DMEM supplemented with about 10% FBS and about 1% penicillin/streptomycin. Translation was inhibited by 200 μg/ml anisomycin. After about a 3-hour incubation, nerve segments were fixed by 4% PFA for about 2 hours and then proceeded to the frozen section.

### Immunohistochemistry

Mice were first perfused by PBS and then 4% PFA for 5 minutes for fixation. Respective tissues were dissected and then post-fixed in 4% PFA for two hours. Fixed tissues were incubated in 30% sucrose overnight and then embedded in an optimum cutting temperature (OCT) compound. Frozen sections were then permeabilized with 0.1% Triton X-100, blocked by 4% normal goat serum for 30 min, and incubated by respective primary antibodies overnight. After PBS washing three times, sections were incubated by secondary antibodies for two hours and washed three times again. Images were taken by Leica SP8 confocal microscope.

### RNA In Situ Hybridization

A commercial kit for RNA-FISH, hybridization chain reaction (HCR) 3.0 was used to assess the localization of IFNγ mRNA in cultured neurons. The probe set was designed by the manufacturer and the RNA-FISH assay was performed according to the manufacturer's protocol. In certain embodiments, cells were fixed by 4% PFA for about 10 minutes. Coverslips were pre-treated by hybridization buffer for about 30 minutes and then incubated with 5 nM probe in 37° C. overnight. B1 HCR amplifier was used for the amplification step. The Tuj 1 staining was performed after amplification. RNA-FISH experiments on tissue sections were performed by using RNAscope Multiplex Fluorescent Reagent Kit v2 according to the manufacturer's protocol. The Tuj 1 staining was performed after TSA amplification.

### Western Blot

DRG, sciatic nerve, or cell pellet was first homogenized and lysed in RIPA buffer for 45 minutes. The RIPA buffer consists of 50 mM Tris-HCl at pH 8.0, 150 mM NaCl, 1% Nonidet P-40, 0.5% Na-deoxycholate, 0.5% SDS. The EDTA-free complete ULTRA tablets (Roche) and PhosSTOP Complete Easypack (Roche) was added to the RIPA lysis buffer before use. The tissue lysis was centrifuged at 10,000 G for 15 minutes. 5×SDS sample buffer [300 mM Tris-HCl buffer, 10% SDS, 5% beta-mercaptoethanol, 50% glycerol, 0.05% bromophenol blue] was mixed with the supernatant and heated to 100° C. for 15 minutes. Western blotting was performed according to the standard protocol that is well known in the art.

### RNA Extraction and qPCR

L4-L6 DRGs or sciatic nerves were dissected out, and RNA extraction was performed by RNeasy Mini Kit according to the manufacturer's protocol, in certain embodiments, the manufacturer can be Qiagen (Hilden, Germany). qPCR was done by the protocol of LightCycler 480 SYBR Green. Three replicates for each sample were used in each run, and Gapdh expression was used as the loading control. Related primers for qPCR are listed below:

| | PCR primer: Ifit1 forward: |
|---|---|
| (SEQ ID NO: 1) | |
| | GTTCTGCTCTGCTGAAAACCC |
| | PCR primer: Ifit1 reverse: |
| (SEQ ID NO: 2) | |
| | CCTGGTCACCATCAGCATTC |
| | PCR primer: Ifi204 forward: |
| (SEQ ID NO: 3) | |
| | TTCCACTGAAGATGGGTGGC |
| | PCR primer: Ifi204 reverse: |
| (SEQ ID NO: 4) | |
| | TCTGGGTTGAGTGGCTTTCC |
| | PCR primer: Mx2 forward: |
| (SEQ ID NO: 5) | |
| | ACCAGAGTGCAAGTGAGGAGCT |
| | PCR primer: Mx2 reverse: |
| (SEQ ID NO: 6) | |
| | GTACTAGGGCAGTGATGTCCTG |
| | PCR primer: Sprr1a forward: |
| (SEQ ID NO: 7) | |
| | GCCTGAAGACCTGATCACCA |
| | PCR primer: Sprr1a reverse: |
| (SEQ ID NO: 8) | |
| | GGTAGCACAAGGCAATGGGA |
| | PCR primer: Gadd45a forward: |
| (SEQ ID NO: 9) | |
| | CTGCAGAGCAGAAGACCGAA |
| | PCR primer: Gadd45a reverse: |
| (SEQ ID NO: 10) | |
| | GGGTCTACGTTGAGCAGCTT |
| | PCR primer: Tubb6 forward: |
| (SEQ ID NO: 11) | |
| | AATGGTGCCCTGGTCTAAGC |
| | PCR primer: Tubb6 reverse: |
| (SEQ ID NO: 12) | |
| | CTGGTCTGCTGGGACTGTTC |
| | PCR primer: Apod forward: |
| (SEQ ID NO: 13) | |
| | GGTGTGGCATGCCTGACTAT |
| | PCR primer: Apod reverse: |
| (SEQ ID NO: 14) | |
| | GCTCACTGTCAGTTTCTCTCAG |
| | PCR primer: Fst forward: |
| (SEQ ID NO: 15) | |
| | TGACAATGCCACATACGCCA |
| | PCR primer: Fst reverse: |
| (SEQ ID NO: 16) | |
| | TTCTTCCGAGATGGAGTTGC |
| | PCR primer: Camk1 reverse: |
| (SEQ ID NO: 17) | |
| | AACTGACCAGGCACAGACG |
| | PCR primer: Camk1 reverse: |
| (SEQ ID NO: 18) | |
| | CCCTAATGTCTTCCGCCTGC |

### RNA Sequencing

For single RGC isolation, 7-8 weeks old WT or Ptpn2 KO mice received IFNγ injection and optic nerve injury. 2 days after injury, micro-Ruby (500 nL, 5% wt/vol. Invitrogen (Waltham, MA)) was gently injected into the optic nerve. The mice were euthanized one day later. The retinas were dissected immediately and digested with 0.5 mg/mL papain for 35 min. Then the fetal bovine serum was added to stop the digestion. After centrifugation and resuspension into Neurobasal A, single RGC was isolated by mouth-pipetting. With a mouth pipette, micro-Ruby positive RGC was pipetted from the original medium drop into a new drop, repeating several times, 5-10 RGCs from one retina were pipetted into a tube containing lysis buffer as a replicate.

cDNA preparation of isolated RGCs or RNA extraction from DRGs was performed by SMART-Seq2 protocol (Picelli et al., 2014). In brief, cells were lysed in 0.2% Triton X-100. Oligo-dT and dNTP were added into the lysis buffer, and the buffer was incubated at 72° C. for 3 minutes. SuperScript™ II (manufactured by Thermo Fisher Scientific (Waltham, MA)) reverse transcriptase and TSO primer were used for reverse transcription. KAPA HiFi HotStart ReadyMix and ISPCR primer were used to amplify cDNA for 25 cycles. cDNA was purified from the PCR product by Ampure XP beads. cDNA was sent to Novogene for Illumina sequencing. Raw reads were first aligned to GRCm38 (mm10) mouse reference genome using STAR (Dobin et al., 2013), and gene counts were calculated using featureCounts. Differential expression genes (DEGs) were assessed with R package DESeq2 (Love et al., 2014). Heatmaps were generated using pheatmap package.

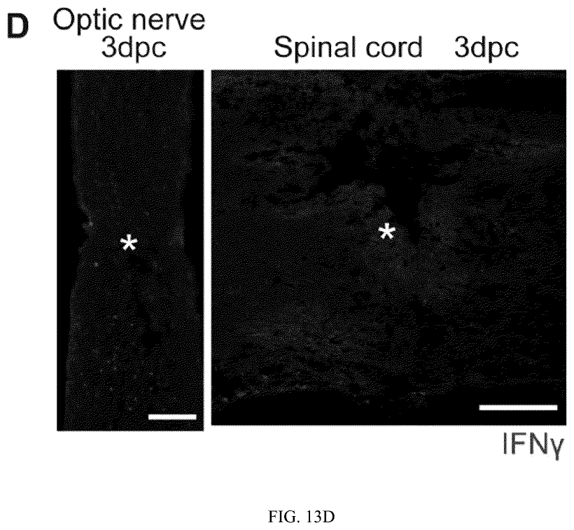

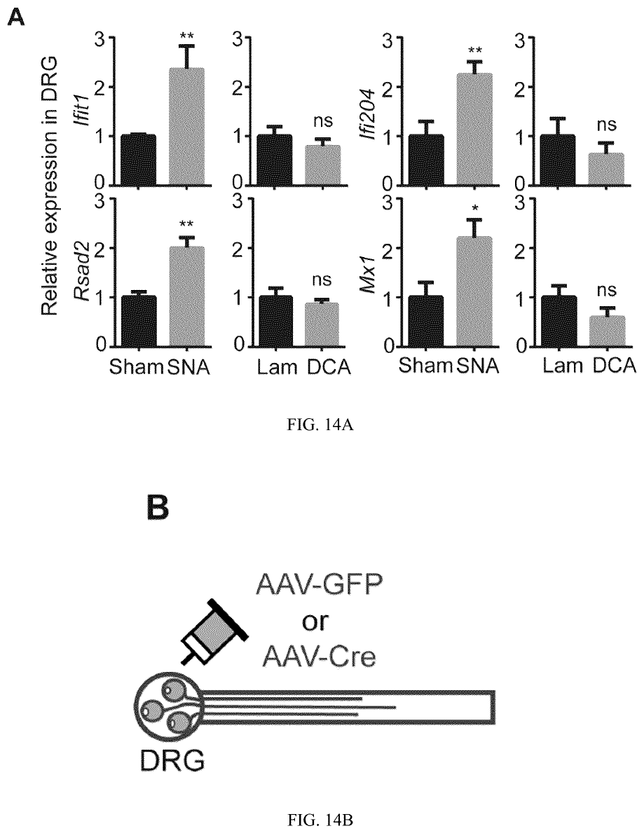

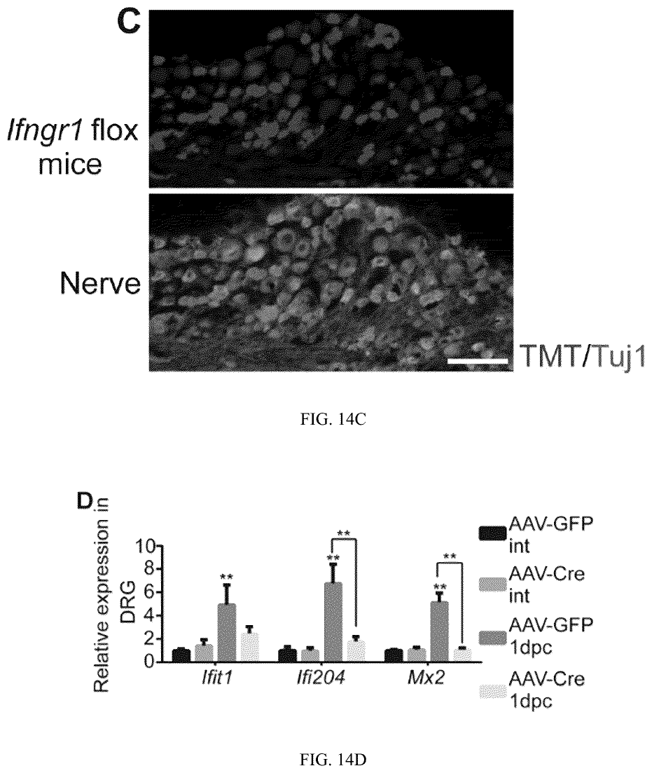

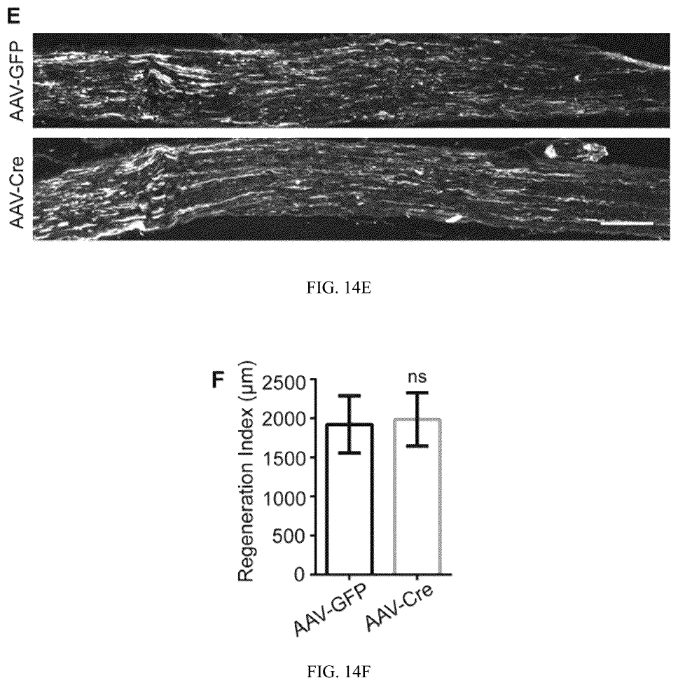

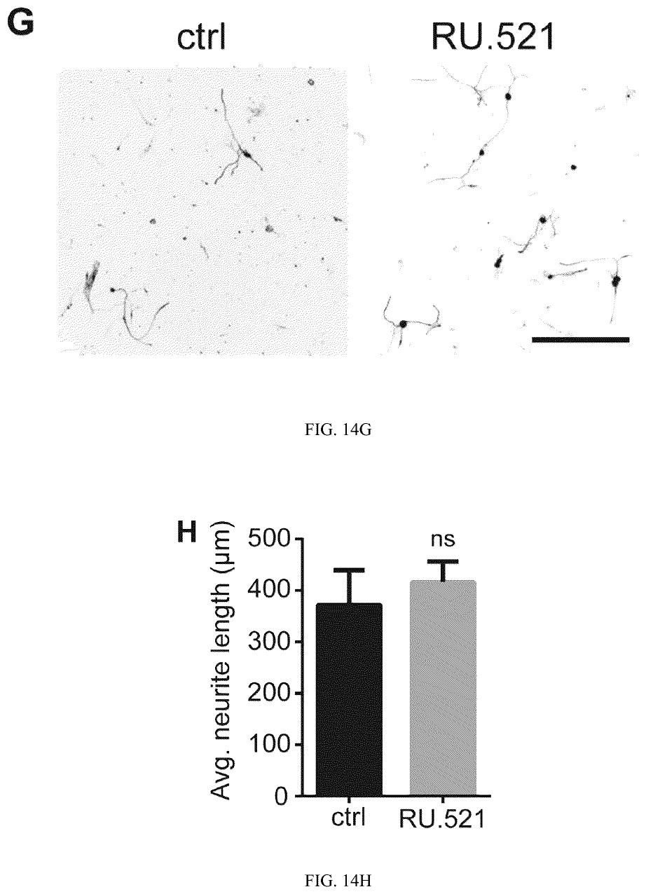

To analyze the previously published dataset (Palmisano et al., 2019), we obtained the fragments per kilobase (FPKM) data of ISGs from Supplementary Dataset 1 attached in the paper and modified the relative expression data by using bar charts in FIG. 14A.

### Quantification and Statistical Analysis

For quantification of neurite lengths in DRG culture. Cells were fixed and stained with TurboGFP (phosphatase shRNA library screening) or Tuj 1. The lengths of the longest axons in each DRG neuron were quantified by the NeuronJ plugin in ImageJ. For phosphatase screening, 5-10 neurons were quantified for each shRNA. For the compound test, 10-20 cells from each mouse and 3 mice were quantified in each group.

To quantify the axon regeneration after sciatic nerve crush, signal intensity from SCG10 immunostaining was measured by ImageJ at different distances from the proximal lesion site. The distance between the lesion site and the column with half the intensity of the lesion site was considered as the regeneration index.

The number of surviving RGCs was determined by whole-mount Tuj 1 staining. The retina was gently exposed and dissected and was washed with 1×PBS 3 times in a 24-well plate. After incubation in 4% normal goat serum (NGS) for 30 mins, the retina was incubated with Tuj 1 antibody overnight at room temperature. After being washed three times with PBS, the retina was incubated with secondary antibodies for one hour at room temperature (about 20° C. to about 22° C.). After being washed with PBS, the retina was mounted onto glass slides. The images were taken by a confocal microscope (Zeiss, LSM 880; 63× objective). Twelve images were taken from the peripheral and central regions of each retina. The number of Tuj 1+cells was counted in a blinded fashion.

To quantify the number of regenerating axons, the sections of optic nerves (thickness t=8 μm) were stained with the FITC antibody and imaged under a confocal microscope (Zeiss, LSM 880; 10X× objective). Five images were taken for quantification from each optic nerve. The number of regenerated axons at indicated distances from the lesion site were estimated by the following formula: Σa_{d}=πr²× [average axon numbers/mm]/t, where r is the radius of the nerve at the quantification site, the mm is the nerve width at the quantification site, the t indicates the section thickness (8 μm). Axon numbers were counted in a blinded fashion.

To quantify the cGAS positive cells in injured sciatic nerves, a 100 μm-by-100 μm square was drawn on sciatic nerve sections at different distance to the lesion center. All the cells (indicated by DAPI staining) within the square were quantified. Cells in intact sciatic nerve sections stained with cGAS antibody were used as the standard of cGAS negative cells. Four nerves were quantified in each group.

All the statistical tests in this experiment were performed using GraphPad Prism 6 software by GraphPad (San Diego, CA). Analysis was performed using a t-test when comparing the mean between two independent groups. Analysis was performed using one-way analysis of variance (ANOVA) followed by Dunnett's or Tukey's post-test as indicated for multiple groups. An estimate of variation is indicated by the standard error of the mean (SEM). **p≤0.01, *p≤0.05.

TABLE 1

| | | |
|---|---|---|
| REAGENT or RESOURCE | SOURCE | IDENTIFIER |

Antibodies

| | | |
|---|---|---|
| Rabbit anti-FITC | Invitrogen | Cat#71-1900; |
| | | RRID: |
| | | AB_2533978 |
| Mouse anti-GAPDH | ProteinTech | Cat#60004-1-Ig; |
| | | RRID: |
| | | AB_2107436 |
| Mouse anti-Tuj1 | BioLegend | Cat#801202; |
| | | RRID: |
| | | AB_10063408 |
| Rabbit anti-Tuj1 | BioLegend | Cat#802001; |
| | | RRID: |
| | | AB_2564645 |
| Rabbit anti-TurboGFP | Evrogen | Cat#AB513 |
| Rabbit anti-IFNγ | Abcam | Cat#ab9657; |
| | | RRID: |
| | | AB_2123314 |
| Chicken anti-NFH | Aves Labs | Cat#NFH; RRID: |
| | | AB_2313552 |
| Chicken anti-GFAP | Aves Labs | Cat#GFAP; RRID: |
| | | AB_2313547 |
| Rabbit anti-cGAS | Cell Signaling | Cat#31659; RRID: |
| | | AB_2799008 |
| Rabbit anti-STING | Cell Signaling | Cat#13647; RRID: |
| | | AB_2732796 |
| Rabbit anti-SCG10 | Novus | Cat#NBP1-49461; |
| | | RRID: |
| | | AB_10011569 |
| Rat anti-CD45 | BioLegend | Cat#103101; |
| | | RRID: |
| | | AB_312966 |
| Rabbit anti-S100β | Cell Signaling | Cat#9550; RRID: |
| | | AB_10949319 |
| Rabbit anti-pSTAT1 | Cell Signaling | Cat#9167; RRID: |
| | | AB_561284 |
| Rabbit anti-STAT1 | Cell Signaling | Cat#14994; RRID: |
| | | AB_2737027 |
| Rabbit anti-pSTAT3 | Cell Signaling | Cat#9145; RRID: |
| | | AB_2491009 |
| Mouse control neutralizing antibody | BioXCell | Cat#BE0089; |
| | | RRID: |
| | | AB_1107769 |
| Mouse IFNGR1 neutralizing antibody | BioXCell | Cat#BE0029; |
| | | RRID: |
| | | AB_1107576 |
| Mouse IFNAR1 neutralizing antibody | BioXCell | Cat#BE0241; |
| | | RRID: |
| | | AB_2687723 |
| Goat anti Mouse 488 | Invitrogen | Cat#A-11029; |
| | | RRID: |
| | | AB_138404 |
| Goat anti Mouse 555 | Invitrogen | Cat#A-21424; |
| | | RRID: |
| | | AB_141780 |
| Goat anti Mouse Cy5 | Invitrogen | Cat#A10524; |
| | | RRID: |
| | | AB_2534033 |
| Goat anti Rabbit 488 | Invitrogen | Cat#A-11008; |
| | | RRID: |
| | | AB_143165 |
| Goat anti Rabbit 555 | Invitrogen | Cat#A-21429; |
| | | RRID: |
| | | AB_2535850 |
| Goat anti Rabbit Cy5 | Invitrogen | Cat#A-10523; |
| | | RRID: |
| | | AB_2534032 |
| Goat anti Chicken 488 | Invitrogen | Cat#A-11039; |
| | | RRID: |
| | | AB_142924 |
| Goat anti Chicken 555 | Invitrogen | Cat#A-21437; |
| | | RRID: |
| | | AB_2535858 |
| Goat anti Chicken 647 | Invitrogen | Cat#A-21449; |
| | | RRID: |
| | | AB_2535866 |

Bacterial and Virus Strains

| | | |
|---|---|---|
| pAAV.hSyn.eGFP.WPRE.bGH | James M. Wilson | Cat#105539; |
| | (unpublished data) | RRID: |
| | | Addgene_105539 |
| pAAV.hSyn.HI.eGFP-Cre.WPRE.SV40 | Penn Vector | Cat#P1848 |
| pAAV.U6.shRLuc.CMV.EGFP.SV40 | Penn Vector | Cat#P1867 |
| pAAV-U6 sgRNA(SapI)_hSyn-GFP-KASH-bGH | (Swiech et al., 2015) | Cat#60958; RRID: |
| | | Addgene_60958 |

Chemicals, Peptides, and Recombinant Proteins

| | | |
|---|---|---|
| Cholera Toxin B Subunit, FITC Conjugate | Sigma-Aldrich | Cat#C1655 |
| IFNγ | Prospec | Cat#cyt-358 |
| leptin | Prospec | Cat#cyt-351 |
| insulin | ThermoFisher | Cat#12585014 |
| EGF | Prospec | Cat#cyt-217 |
| NGF | Prospec | Cat#cyt-579 |
| RU.521 | MedChemExpress | Cat#HY-114180 |
| C-176 | MedChemExpress | Cat# HY-112906 |
| H-151 | MedChemExpress | Cat# HY-112693 |
| 2′, 3′-cGAMP | MedChemExpress | Cat#HY-100564A |
| DMXAA | MedChemExpress | Cat# HY-10964 |
| ADU-S100 | MedChemExpress | Cat#HY-12885A |
| Anisomycin | Sigma-Aldrich | Cat# A9789 |
| Paraformaldehyde (PFA) | Sigma-Aldrich | Cat#30525-89-4 |
| Optimal Cutting Temperature compound | SAKURA | Cat#4583 |
| Triton X-100 | Sigma-Aldrich | Cat#T8787 |
| Normal goat serum | Invitrogen | Cat#50062Z |
| Neurobasal A | Gibco | Cat#10888022 |
| Neurobasal | Gibco | Cat#21103049 |
| Leibovitz's L-15 medium | Gibco | Cat#21083027 |
| TrypLE | Gibco | Cat#12604021 |
| GlutaMAX | Gibco | Cat#35050061 |
| 5-fluoro-2′-deoxyuridine | Sigma | Cat#F0503 |
| B27 supplement | Gibco | Cat#17504044 |
| penicillin-streptomycin | Gibco | Cat#15140122 |
| Fetal bovine serum | HyClone | Cat#SH3007103 |
| DMEM | Gibco | Cat#12800017 |
| Papain | Sigma-Aldrich | Cat#P4762 |
| DMSO | Sigma-Aldrich | Cat#D2650 |
| Sucrose | Invitrogen | Cat#15503022 |
| DAPI | Sigma-Aldrich | Cat#D9542 |
| Collagenase | Roche | Cat#11088858001 |
| Laminin | Gibco | Cat#23017015 |
| PhosSTOP | Roche | Cat#04906837001 |
| cOmplete Tablets | Roche | Cat#05892791001 |

Critical Commercial Assays

| | | |
|---|---|---|
| Mouse Neuron Nucleofector Kit | Lonza | Cat#VPG-1001 |
| HCR 3.0 RNA-FISH Kit | Molecular | N/A |
| | Instrument | |
| RNAScope Multiplex Fluorescence V2 | ACDBio | Cat#323133 |
| KAPA HiFi HotStart ReadyMix | Roche | Cat#KK2601 |
| SuperScript II Reverse Transcriptase | Invitrogen | Cat#18064014 |
| LightCycler 480 SYBR Green I Master | Roche | Cat#04707516001 |
| RNAeasy mini Kit | QIAGEN | Cat#74104 |

Experimental Models: Cell Lines

| | | |
|---|---|---|
| SpCas9 Neuro2a | GeneCopoeia | Cat#SL509 |

Experimental Models: Organisms/Strains

| | | |
|---|---|---|
| Mouse: Adult C57B16/J | Charles River | N/A |
| Mouse: Ptpn2 flox | This paper | N/A |
| Mouse: Ptpn2^{tmla(EUCOMM)Wtsi} | International Mouse | Cat#: MGI97806 |
| | Phenotyping | |
| | Consortium | |
| Mouse: Jak1 flox | This paper | N/A |
| Mouse: Stat3 flox | (Moh et al., 2007) | Cat#: 016923; |
| | | RRID: |
| | | IMSR_JAX: 016923 |
| Mouse: Mtor flox | (Risson et al., 2009) | Cat#: 011009; |
| | | RRID: |
| | | IMSR_JAX: 011009 |
| Mouse: Ifngr1 flox | (Lee et al., 2013) | Cat#025394; |
| | | RRID: |
| | | IMSR_JAX: 025394 |
| Mouse: Pten/Socs3 flox | (Sun et al., 2011) | N/A |
| Mouse: Advillin-CreER | (Lau et al., 2011) | Cat#032027; |
| | | RRID: |
| | | IMSR_JAX: 032027 |
| Mouse: Sting knockout | (Jin et al., 2011) | Cat#025805; |
| | | RRID: |
| | | IMSR_JAX: 025805 |
| Mouse: Cgas knockout | (Schoggins et al., | Cat#026554; |
| | 2014) | RRID: |
| | | IMSR_JAX: 026554 |
| Mouse: Mavs knockout | (Sun et al., 2006) | Cat#008634; |
| | | RRID: |
| | | IMSR_JAX: 008634 |
| Mouse: Vglut2-Cre | (Vong et al., 2011) | Cat#028863; |
| | | RRID: |
| | | IMSR_JAX: 028863 |
| Mouse: Rosa26-LSL-Cas9 knockin | (Platt et al., 2014) | Cat#026175; |
| | | RRID: |
| | | IMSR_JAX: 026175 |
| Mouse: Rosa26-LSL-TMT knockin | (Madisen et al., | Cat#: 007905; |
| | 2010) | RRID: |
| | | IMSR_JAX: 007905 |
| Mouse: Thy1-GFP line M | (Feng et al., 2000) | Cat#007788; |
| | | RRID: |
| | | IMSR_JAX: 007788 |

Oligonucleotides

| | | |
|---|---|---|
| shRNA and sgRNA sequence: Tables 1 and 2 | This invention | N/A |
| PCR primers: Ifit1 forward: | This invention | N/A |
| GTTCTGCTCTGCTGAAAACCC (SEQ ID NO: 1) | | |
| PCR primers: Ifit1 reverse: | This invention | N/A |
| CCTGGTCACCATCAGCATTC (SEQ ID NO: 2) | | |
| PCR primers: Ifi204 forward: | This invention | N/A |
| TTCCACTGAAGATGGGTGGC (SEQ ID NO: 3) | | |
| PCR primers: Ifi204 reverse: | This invention | N/A |
| TCTGGGTTGAGTGGCTTTCC (SEQ ID NO: 4) | | |
| PCR primers: Mx2 forward: | This invention | N/A |
| ACCAGAGTGCAAGTGAGGAGCT (SEQ ID NO: 5) | | |
| PCR primers: Mx2 reverse: | This invention | N/A |
| GTACTAGGGCAGTGATGTCCTG (SEQ ID NO: 6) | | |

Software and Algorithms

| | | |
|---|---|---|
| Image J | https://imagej.nih.gov/ij/ | RRID: SCR_002285 |
| Prism 6 | https://www.graphpad.com/ | N/A |
| RStudio | https://www.rstudio.com/ | N/A |
| R | https://www.r-project.org/ | RRID: SCR_001905 |

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### RESULTS

### Example 1—PTPN2 Inhibits Axon Regeneration

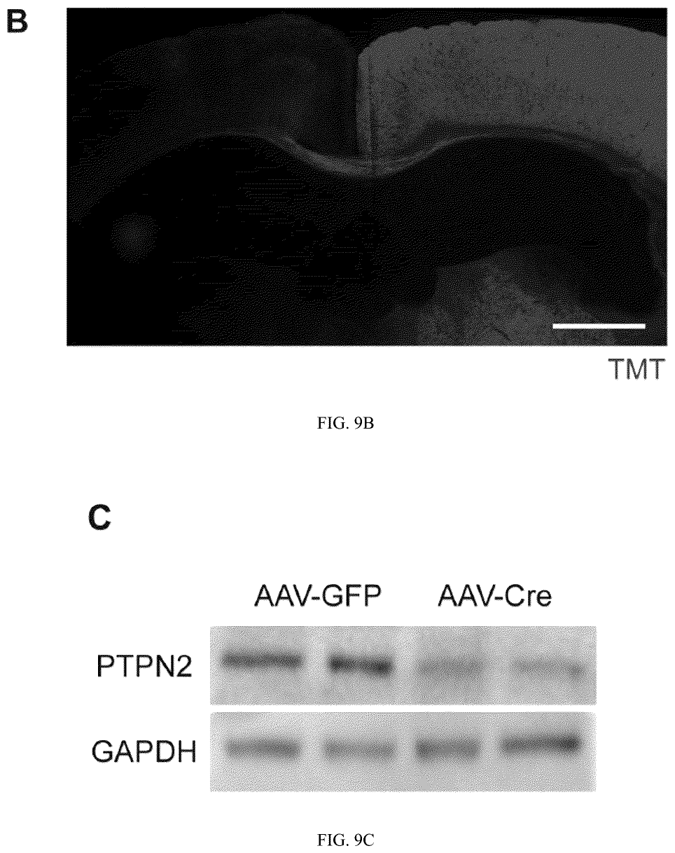

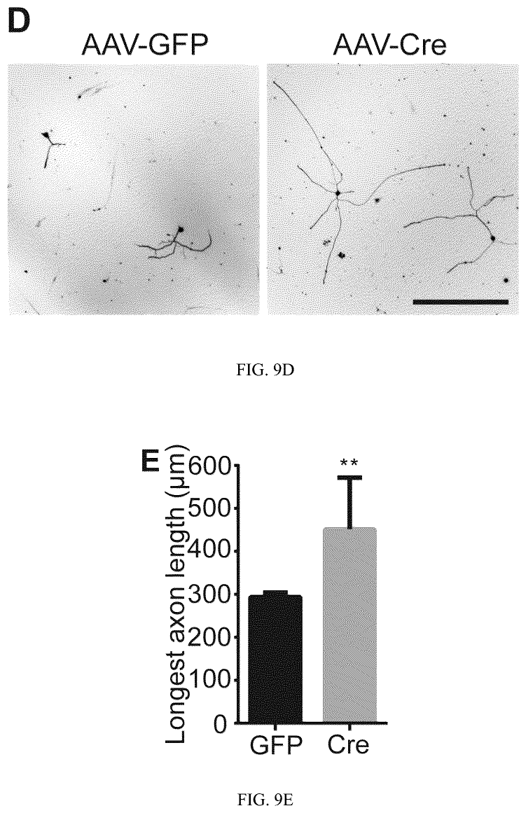

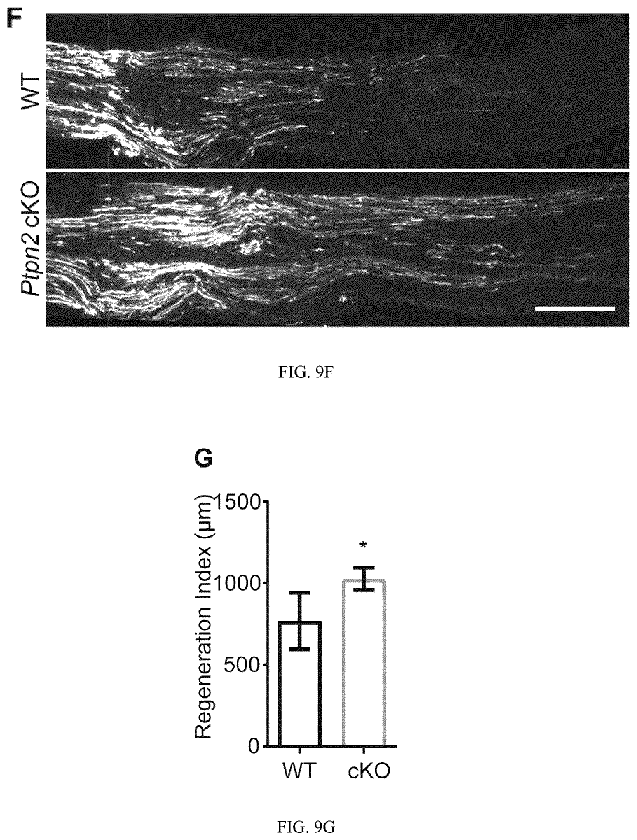

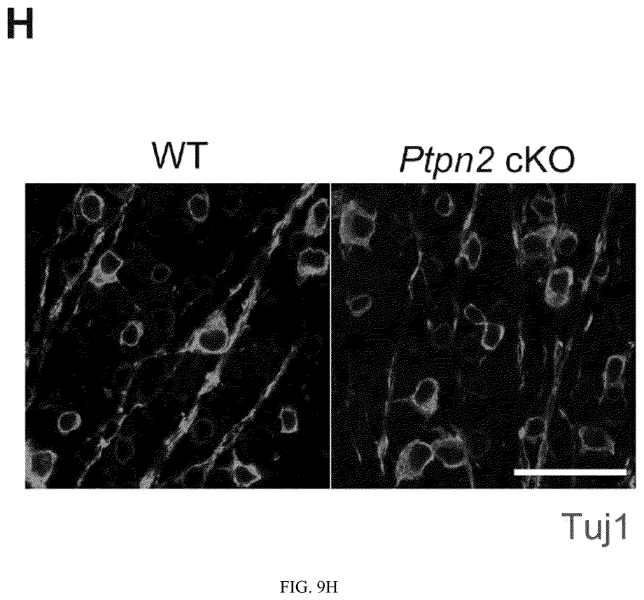

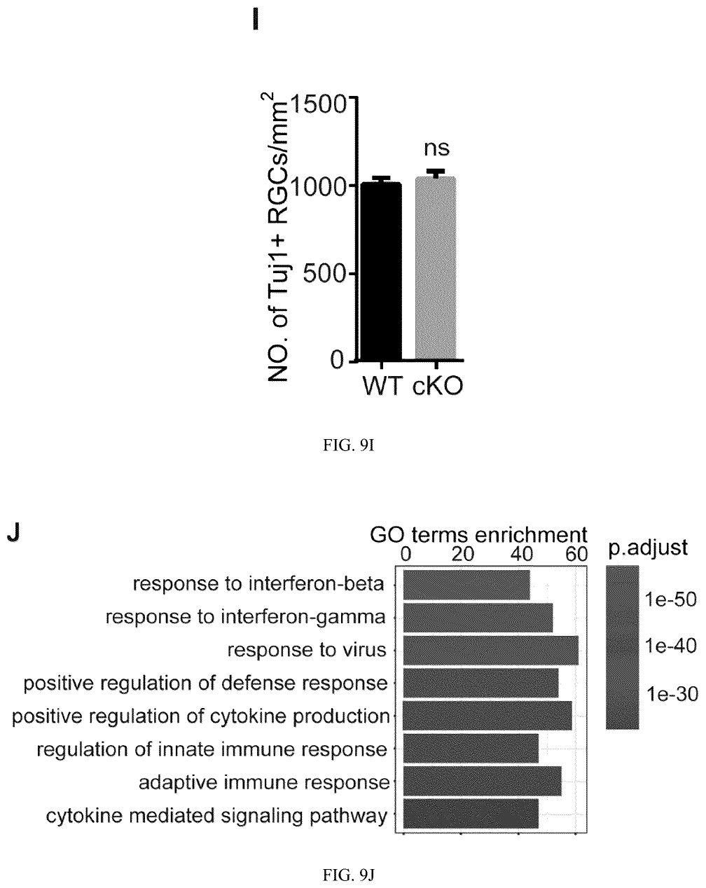

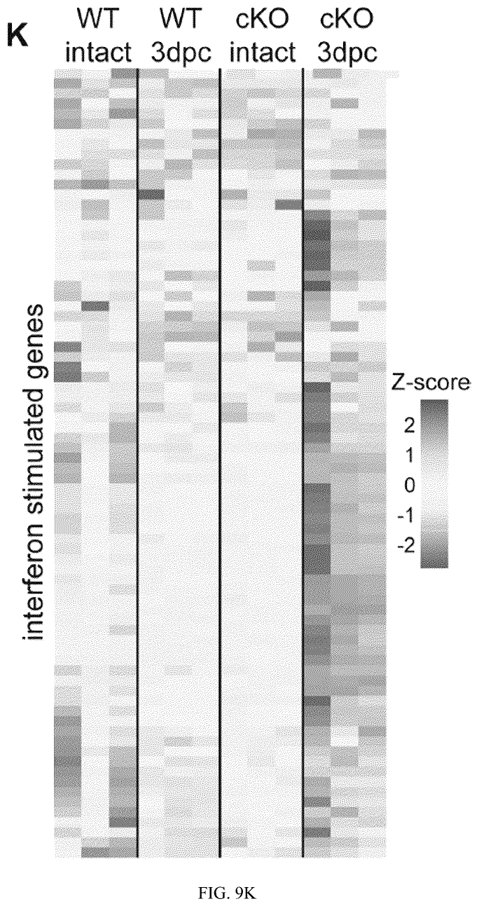

We used the DRG replating assay as a screening system to search for intrinsic suppressors of axon growth (Frey et al., 2015; Saijilafu et al., 2013). Several phosphatases have been identified as suppressors of axon regeneration (Fisher et al., 2011; Park et al., 2008; Shen et al., 2009), and they are also viable targets for therapeutic development (Zhang, 2017). To identify phosphatases that inhibit axon elongation, we collected a list through the Dharmacon mouse shRNA library (Waterbeach, United Kingdom) targeting phosphatases, checked the gene expression using the in situ Allen brain atlas, and arrived at about 84 genes that could be expressed in the brain. We knocked down individual genes using short hairpin RNA (shRNA) in cultured adult dorsal root ganglion (DRG) neurons and evaluated the resulting effect on axon elongation. We found that five phosphatase genes inhibited axon growth, including previously identified Pten and Ptprf (FIGS. 1A-1B). We conducted a small-scale screening using a panel of specific protein tyrosine phosphatase inhibitors (Zhang, 2017 and unpublished). PTPN2 was also identified from the tyrosine phosphatase inhibitor screening (FIGS. 1C-1D). We subsequently used the optic nerve injury model to assess the effect of PTPN2 inhibition in the CNS in vivo. Directly injecting the PTPN2 inhibitor (Zhang et al., 2009) into the vitreous humor of adult wild-type (WT) mice elicited modest axon regeneration at two weeks after optic nerve crush in a dose-dependent manner (FIGS. 1E-1F). To confirm, we generated Ptpn2 floxed mice (FIGS. 9A). The knockout efficacy was verified by injecting adeno-associated virus carrying Cre recombinase (AAV-Cre) into the neonatal mouse cortex and performing western blotting of the cortical tissue (FIGS. 9B-9C). Consistent with the shRNA transfection result, the deletion of Ptpn2 from DRG neurons through the addition of AAV-Cre into the culture increased the axon elongation (FIGS. 9D and 9E). Next, we generated tamoxifen-inducible conditional knockout of Ptpn2 in DRG neurons by crossing Advillin-CreERT2 with Ptpn2 foxed mice (Ptpn2 cKO) for in-vivo experiments. Then we knocked out Ptpn2 in adult dorsal root ganglion neurons and assessed the peripheral axon regeneration after sciatic nerve crush. Ptpn2 deletion promoted peripheral sensory axon regeneration at 2 days after crush (FIGS. 9F-9G). Next, we injected AAV-hSyn-Cre into the eyes of adult Ptpn2 foxed mice to induce conditional knockout (KO) in RGCs. Then, we performed optic nerve crush and assessed axon regeneration two weeks post injury. We found RGC survival rates to be comparable between the control and KO groups (FIGS. 9H-9I). Cholera toxin b subunit (CTB) labeling of the optic nerves showed modestly more regenerating axons in mice injected with AAV-Cre than in the control mice (FIGS. 1G-1H). The results from the PTPN2 inhibitor and KO experiments indicated that PTPN2 plays an inhibitory role in axon regeneration in vivo.

### Example 2—IFNγ Boosts Ptpn2 cKO Induced Axon Regeneration and Synergizes with Pten/Socs3 Codeletion

PTPN2 negatively regulates several cellular signaling pathways, such as, for example, hormones, growth factors, and cytokines, by dephosphorylating receptor and non-receptor protein tyrosine kinases. We speculated that Ptpn2 deletion may promote axon growth by interacting with injury signals, and then sought to explore the transcriptional effect of Ptpn2 deletion in injured neurons. We designed an RNA-seq experiment comparing the transcriptomes in four different groups of DRGs (WT intact, WT 3dpc, Ptpn2 cKO intact and Ptpn2 cKO 3dpc). To elucidate which signaling pathway was activated in Ptpn2 cKO DRGs after injury, we first identified the differentially expressed genes (DEG, fold change>2, p adjust<0.1) in cKO 3dpc group by comparing with the WT 3dpc group. Gene Ontology analysis showed that the upregulated genes were significantly enriched in the categories related to host defense response and interferon-related signaling (FIG. 9J). According to previous study, interferon stimulated genes (ISGs) are the downstream genes upregulated by interferon stimulation (Hubel et al., 2019; Schneider et al., 2014; Schoggins, 2019). To directly check the expression level of these genes, we searched for these ISGs in our transcriptomic data. We found that 39 of 78 ISGs expressed in DRGs were upregulated in cKO injury compared to WT injury. However, only 6 of 78 ISGs were upregulated in cKO intact compared to WT intact (FIG. 9K). Ptpn2 deletion alone did not enhance the expression of ISGs before injury. This result suggested that Ptpn2 deletion interacted with injury signals to enhance ISG signaling.

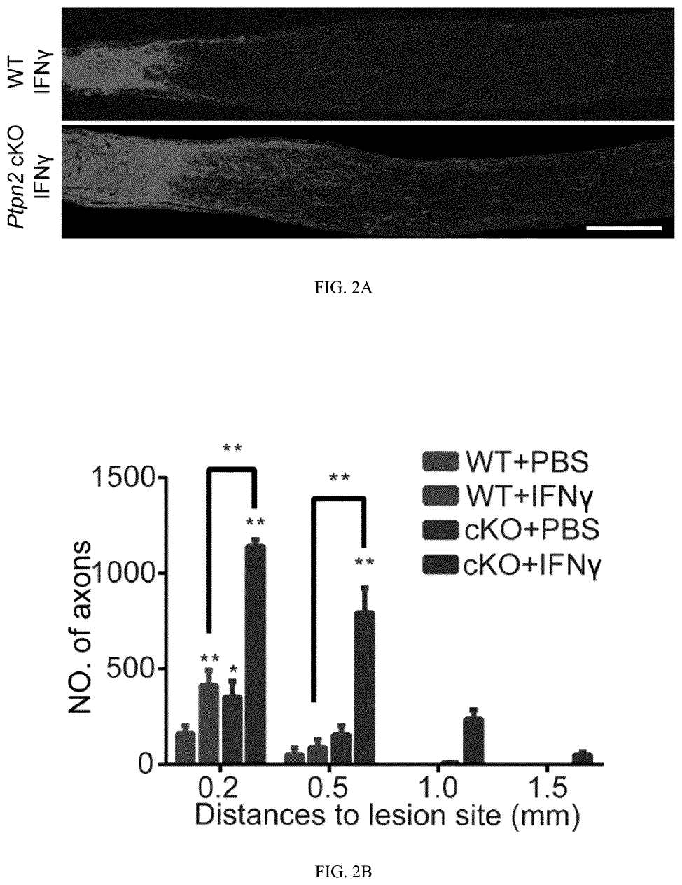

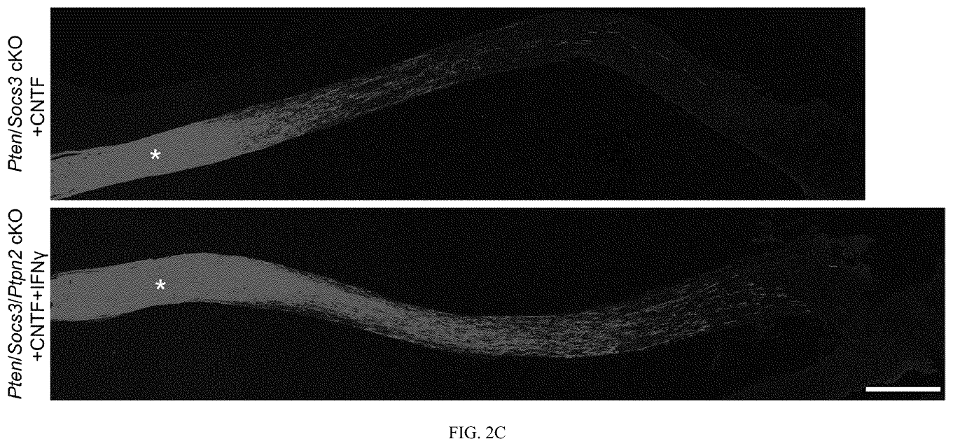

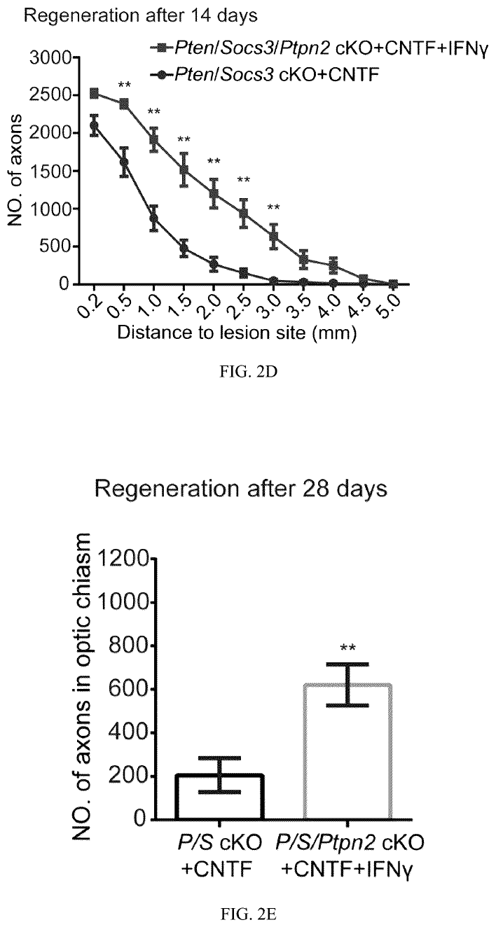

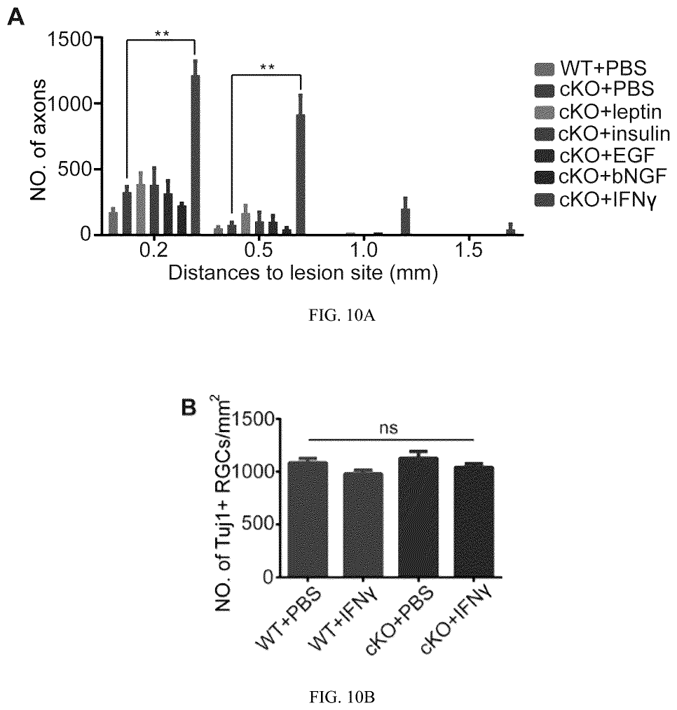

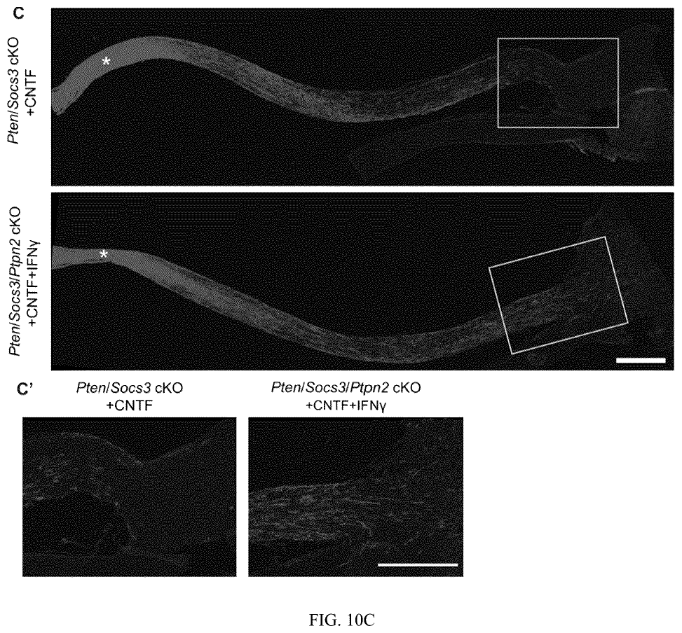

To assess whether exogenous ligands can further enhance the axon regeneration induced by Ptpn2 deletion, we injected individual candidate recombinant proteins or peptides into the eyes of Ptpn2 KO mice at the time of optic nerve crush. In two weeks, low-dose IFNγ, but not leptin, insulin, EGF, or NGF, significantly boosted axon regeneration (FIG. 10A). We repeated the experiment by including more control groups, and found that while IFNγ alone induced modest regrowth, IFNγ plus Ptpn2 KO elicited robust regeneration (FIGS. 2A-2B). The RGC survival rates were comparable among the four groups (FIG. 10B). Then, we wondered whether this strategy could further enhance the regrowth induced by Pten/Socs3 codeletion, by which not only long-distance axon regeneration but also synaptic reconnection has been shown after optic nerve or optic tract lesions (Bei et al., 2016; Li et al., 2015; Sun et al., 2011). To test this idea, we injected AAV-Cre into the vitreous body of Pten/Socs3 double floxed or Pten/Socs3/Ptpn2 triple floxed mice to generate double or triple knockout of the floxed genes in RGCs, together with AAV-CNTF. At the time of injury, we injected vehicle into the Pten/Socs3 mutant and IFNγ into the Pten/Socs3/Ptpn2 mutant. At two weeks after injury, CTB labeling showed a very significant increase in axon regeneration in the triple KO group (FIG. 2C). A synergistic effect was observed at 1 mm or longer distal to the lesion site (FIG. 2D). The enhanced growth continued at four weeks, and about 3-fold more axons could be found at the optic chiasm (FIGS. 2E, FIG. 10C). Thus, IFNγ boosted the Ptpn2 cKO effect and synergized with Pten/Socs3 codeletion.

### Example 3—Neuronal Ptpn2 Deletion Amplifies IFNγ Response and Sustains STAT1 Activation to Promote Axon Regeneration

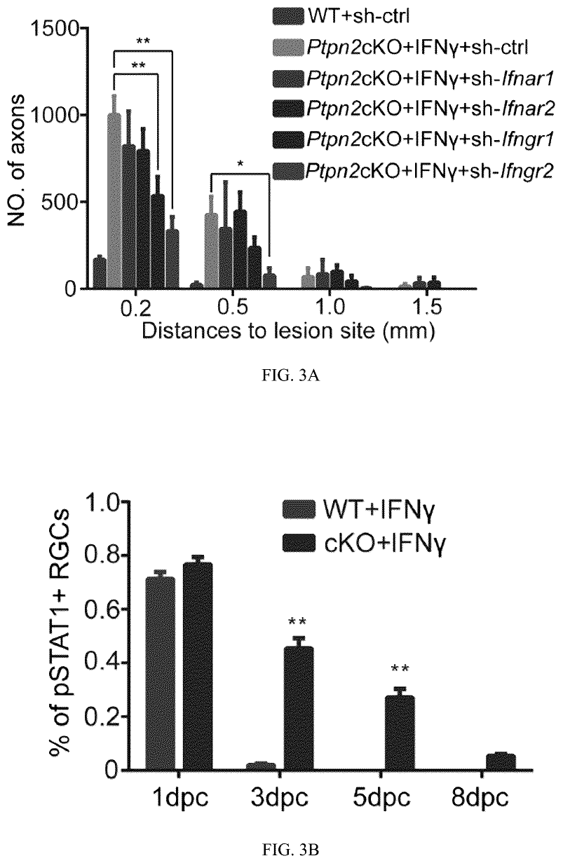

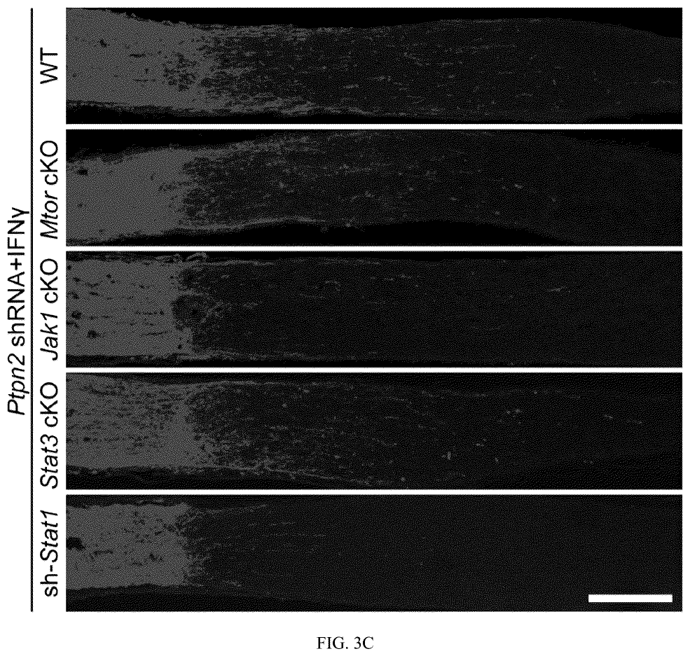

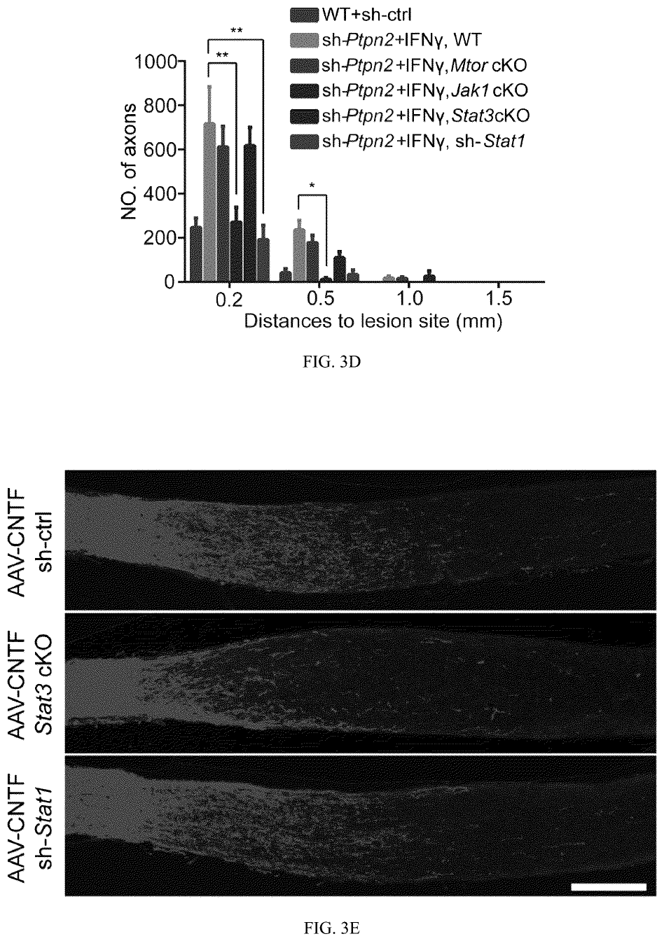

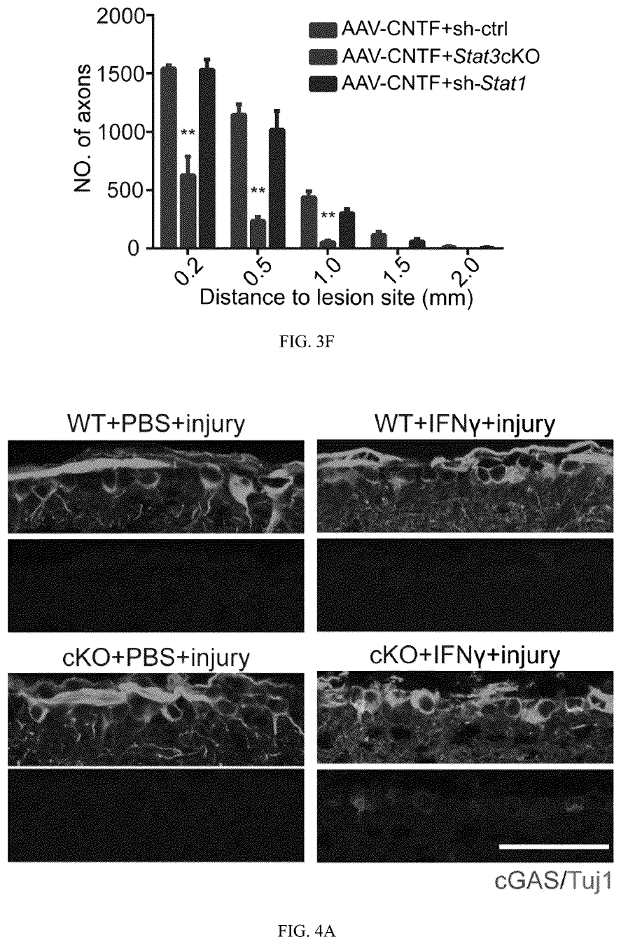

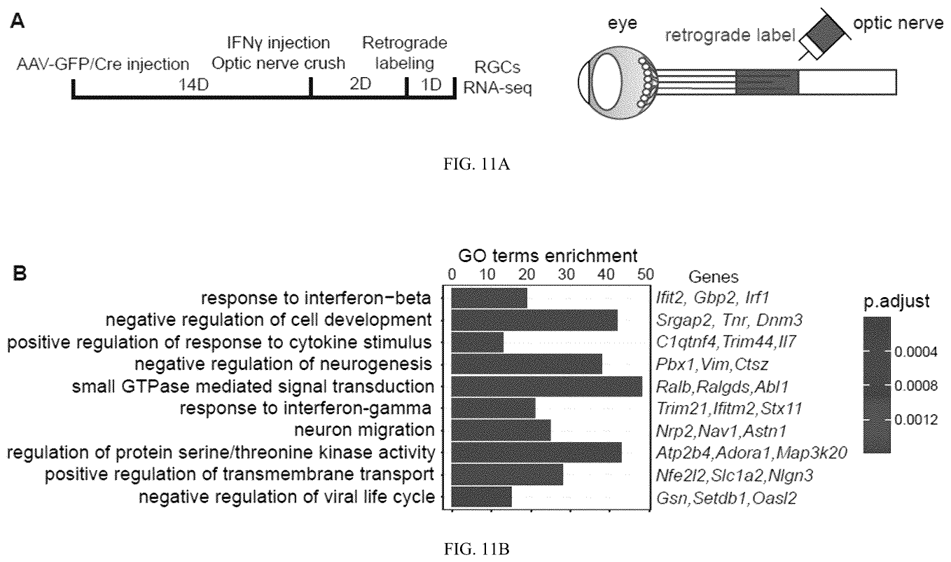

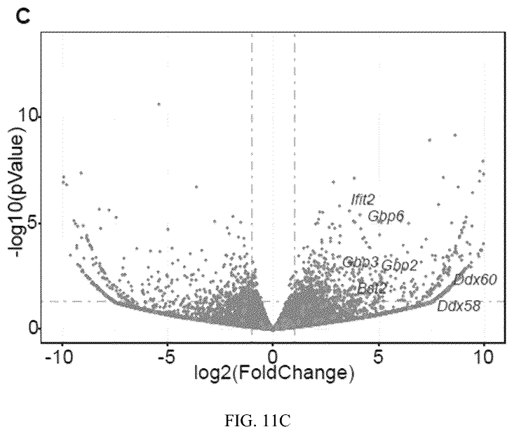

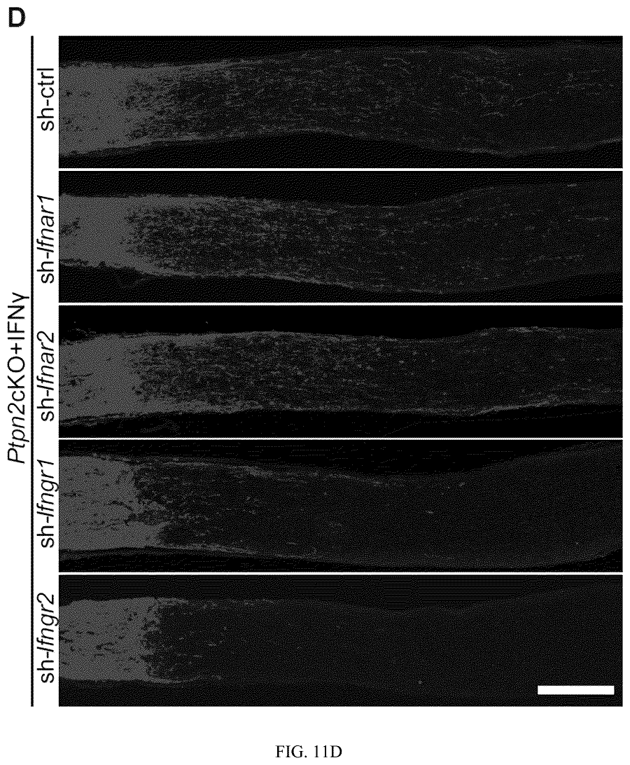

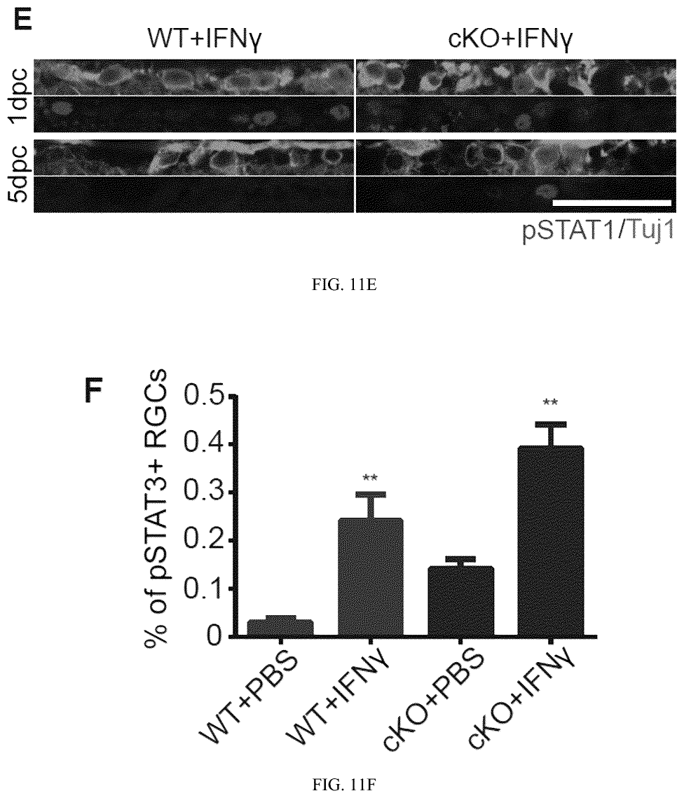

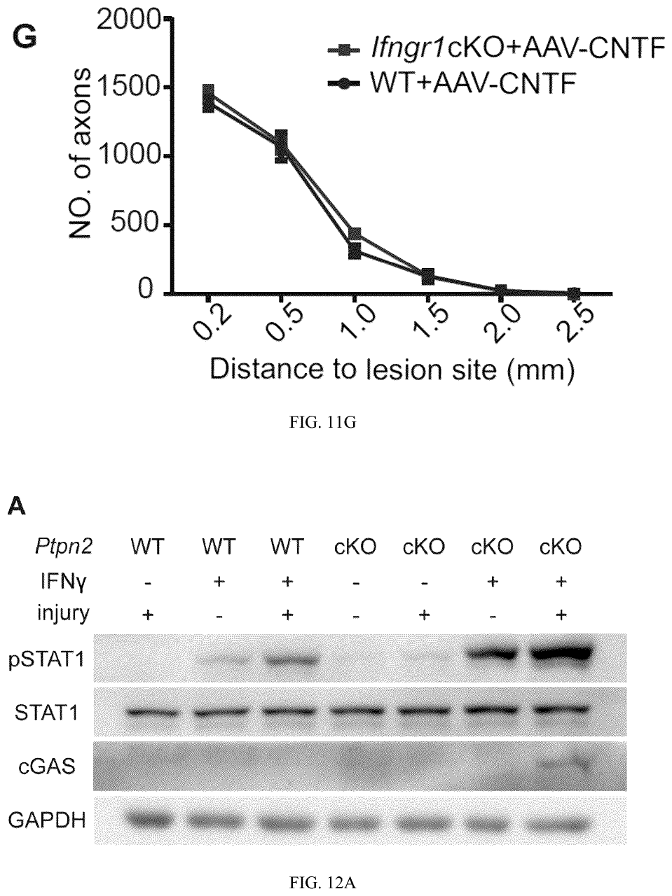

To investigate the mechanism by which IFNγ stimulated axonal regeneration of RGCs upon Ptpn2 deletion, we performed retrograde labeling of RGCs, isolated RGCs by cell sorting, and conducted RNA-Seq (FIG. 11A) at three days after injury. Analysis showed that interferon response genes were among the most prominently affected (FIG. 11B). ISGs are commonly expressed in the immune cells in response to viral infection, and our data showed that ISGs could also be induced in RGCs (FIG. 11C). It is known that the type II interferon IFNγ activates cellular responses through its interactions with a heterodimeric receptor consisting of interferon gamma receptor 1 (IFNGR1) and interferon gamma receptor 2 (IFNGR2); both receptors are required to elicit the full function of IFNγ signaling. Thus, we evaluated whether the growth effect induced by Ptpn2 KO plus IFNγ was IFNGR-dependent. We generated AAVs expressing either Ifngr1 shRNA (sh-Ifngr 1) or Ifngr2 shRNA (sh-Ifngr2) and injected them into mouse eyes to decrease the level of the IFNGR complex in RGCs. In mice with Ptpn2 KO plus IFNγ, Ifngr1 or Ifngr2 KD evidently suppressed the enhanced axon regeneration of the injured optic nerve (FIG. 3A and FIG. 11D). We also included AAVs carrying Ifnar1 shRNA (sh-Ifnar1) or Ifnar2 shRNA (sh-Ifnar2) as controls, since interferon-α/β receptor subunit 1 (IFNAR1) and interferon-α/β receptor subunit 2 (IFNAR2) form the interferon-α/β receptor (IFNAR) complex that is activated by type I interferons (IFN-I) but not by IFNγ. As expected, Ifnar1 and Ifnar2 KD did not affect the regrowth (FIG. 3A and FIG. 11D). This data suggests that IFNγ stimulates axon regeneration specifically through its ligand-receptor interaction. Thus, neuronal Ptpn2 deletion amplifies the IFNγ response in RGCs to promote axon regeneration.

Next, we examined how downstream signaling is amplified and contributes to the axon regeneration induced by Ptpn2 KO plus IFNγ. Canonically, IFNGR engaged by IFNγ leads to the activation of JAK1 and JAK2 and the subsequent phosphorylation of STAT1 and STAT3. Phosphorylated STAT1 and STAT3 translocate to the nucleus and stimulate the transcription of target genes. By performing immunostaining, we found that phosphorylated STAT1 (p-STAT1) was barely detectable in WT RGCs. IFNγ administration induced p-STAT1 accumulation in the nuclei of RGCs at 1 day post crush (dpc) but not at 3 dpc. In contrast, p-STAT1 was enhanced up to 5 dpc in the Ptpn2 KO group (FIG. 3B and FIG. 11E); phosphorylated STAT3 (p-STAT3) was also activated (FIG. 11F). It has been shown that GP130-STAT3 signaling mediates the regeneration induced by CNTF (Smith et al., 2009; Sun et al., 2011), and mTOR signaling is the central mediator of axon regeneration induced by Pten deletion (Park et al., 2008). To determine which STAT(s) can mediate the growth and whether this effect has any crosstalk with mTOR, we carried out loss-of-function experiments using Ptpn2 shRNA combined with either conditional KO for Jak1, Stat3, mTOR, or shRNA knockdown for Stat1. We found that either Jak1 cKO or Stat1 KD almost completely blocked the regeneration, but neither Stat3 cKO, nor mTOR cKO showed any significant suppression of the regrowth (FIG. 3C and FIG. 3D). Then, we evaluated whether IFNγ signaling is involved in the regeneration induced by AAV-CNTF. Neither Stat1 KD (FIG. 3E and FIG. 3F), nor Ifngr deletion (FIG. 11G) decreased the regeneration induced by AAV-CNTF, which was largely suppressed by Stat3 cKO (FIGS. 3E and 3F).

In conclusion, Ptpn2 KO plus IFNγ promotes axon regeneration mainly through the IFNGR-JAK1-STAT1 signaling axis, distinct from the STAT3 or mTOR pathway; therefore, sustained STAT1 activation mediates the regeneration. The combination of three independent mechanisms likely contributes to the observed synergy produced by the triple knockout of Pten, Socs3, and Ptpn2.

### Example 4—the cGAS-Sting Pathway is Essential for the Regeneration Induced by Ptpn2 Deletion Plus IFNγ

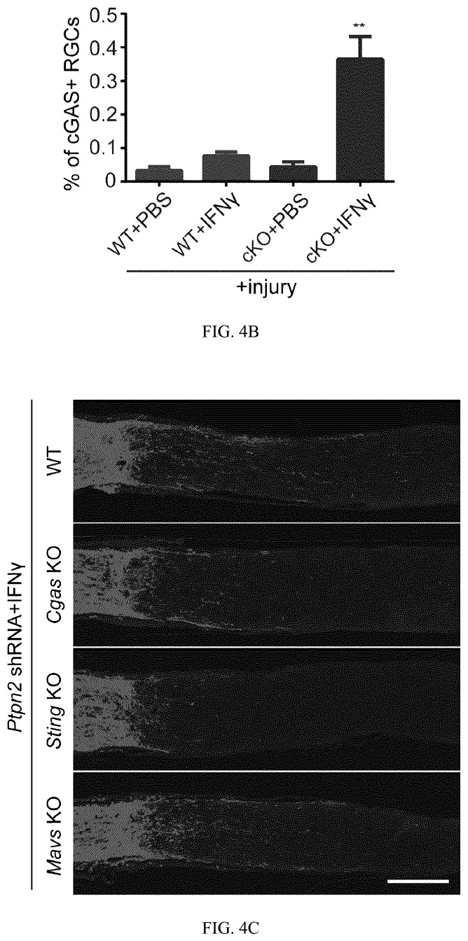

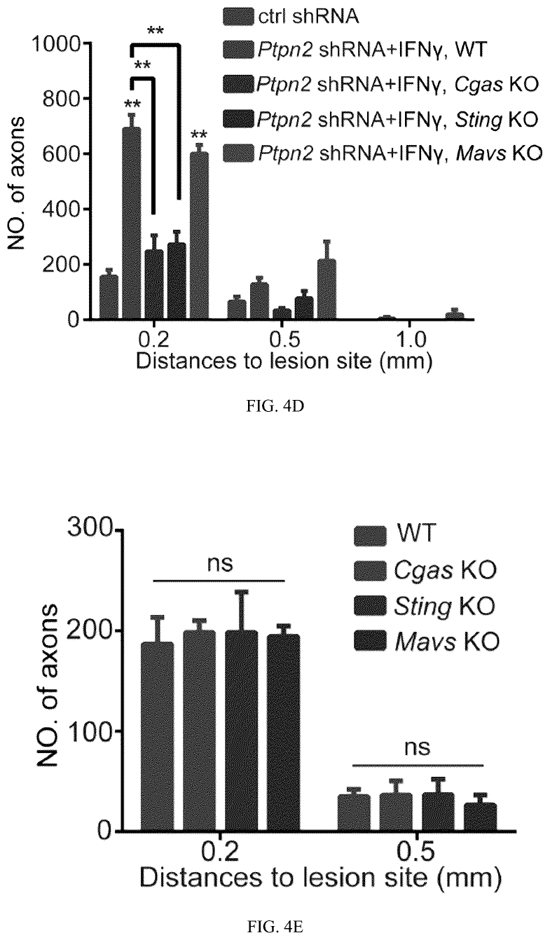

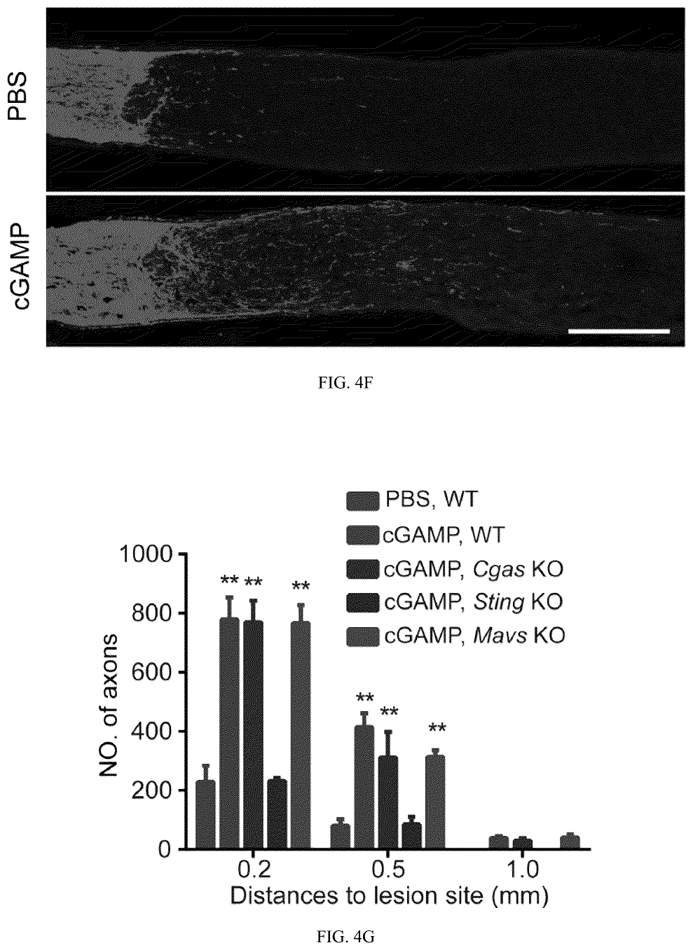

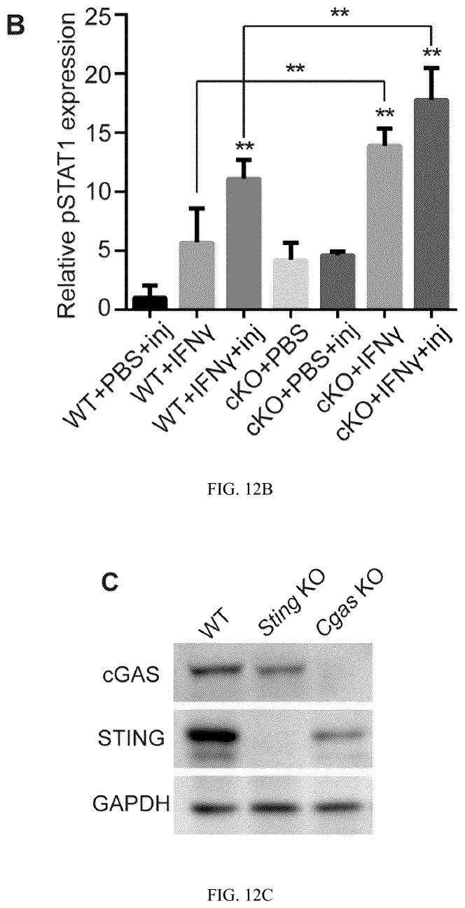

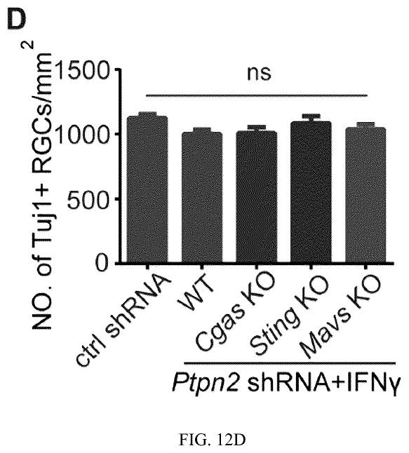

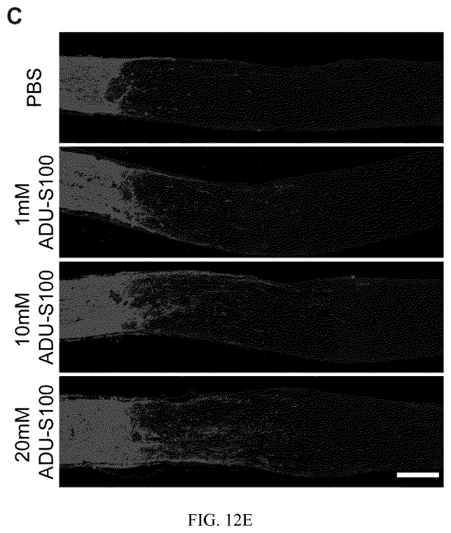

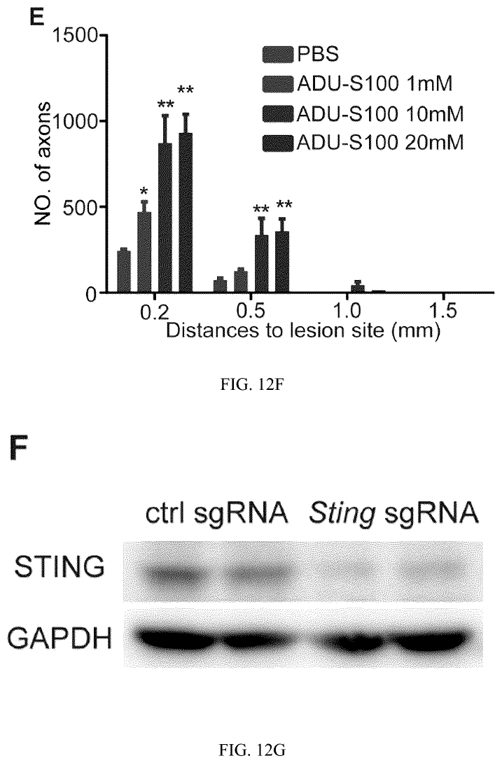

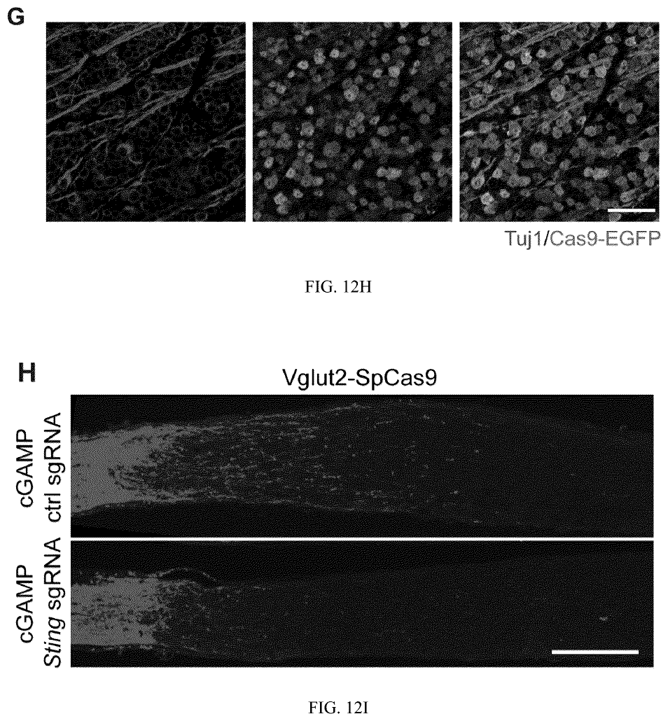

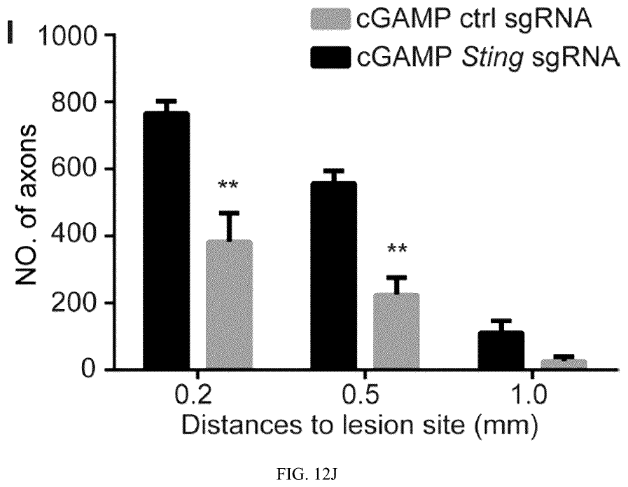

In some embodiments, STAT1 activation plays a critical role in regeneration and is consistent with the RNA-seq data we collected on the upregulation of many ISGs. We speculated that ISGs could be functionally linked with the regeneration and investigated which specific ISGs may be downstream effectors. In some macrophages, PTPN2 negatively regulates the cGAS-STING pathway (Xia et al., 2019). cGAS and STING are also ISGs transcriptionally induced by interferon-STAT1 activation through a positive feedback mechanism (Ma et al., 2015a; Ma et al., 2015b). After sensing cytosolic double-strand DNA, cGAS is activated and catalyzes the production of the second messenger cGAMP, which binds to and activates STING (Ablasser and Chen, 2019). We examined whether cGAS-STING signaling is involved in the regeneration effect. By performing western blotting, we found that cGAS was detectable only in mouse retinas with Ptpn2 cKO plus IFNγ after optic nerve injury (FIG. 12A), when p-STAT1 was most elevated among all groups (FIG. 12B). With immunostaining, we verified that cGAS was upregulated in RGCs at 2 days after injury in mice with Ptpn2 cKO in addition to IFNγ, but not in the PBS, IFNγ-only, or Ptpn2-cKO-only group (FIGS. 4A and 4B). This result indicates that the injury signal is required to induce cGAS. Next, we injected AAV-shRNA-Ptpn2 into Cgas, Sting (FIG. 12C), or Mays (mitochondrial antiviral signaling protein) of KO mice to knock down Ptpn2 in RGCs and assess the optic nerve regeneration by IFNγ stimulation. Distinct from STING, MAVS is an adaptor protein that transduces signals from cytosolic RNA sensors. At 2 weeks after injury, axon regeneration was almost completely blocked in Cgas or Sting KO mice but not in Mays KO mice (FIGS. 4C and 4D). RGC survival was comparable among all groups (FIG. 12D). None of the KOs alone affected the axon regeneration (FIG. 4E).

To test whether activating the STING pathway can promote axon regeneration, we directly injected 2′,3′-cGAMP into the mouse eye and performed the optic nerve crush. STING ligand 2′,3′-cGAMP is a cyclic dinucleotide (CDN) produced in mammalian cells, and exogenous 2′,3′-cGAMP can be transported into cells and activate STING. 2 weeks after injury, 2′,3′-cGAMP stimulated significant axon regeneration (FIG. 4F), which was almost completely blocked in Sting KO mice but not in Cgas or Mays KO mice (FIG. 4G). The synthetic CDN ADU-S100, which is under clinical trials for cancer immunotherapy, also showed a regenerative effect in a dose-dependent manner (FIG. 12F). To assess whether neuronal STING is essential for the regeneration induced by cGAMP, we crossed Vglut2-cre mice with cre-dependent SpCas9 mice (Vglut2-SpCas9) to express Cas9 in excitatory neurons and injected AAV-sgRNA into the eye to knock out STING in RGCs (FIGS. 12G and 12J). We found that cGAMP-induced regeneration was suppressed in Vglut2-SpCas9 mice injected with AAV-sgRNA-Sting but not in those injected with AAV-sgRNA-ctrl (FIGS. 12I-12J). Thus, through these loss- and gain-of-function experiments, we demonstrated that neuronal cGAS-STING signaling in RGCs plays a critical role in mediating the axon regeneration induced by Ptpn2 deletion plus IFNγ.

### Example 5—Axonal IFNγ is Locally Translated after Peripheral Axon Injury

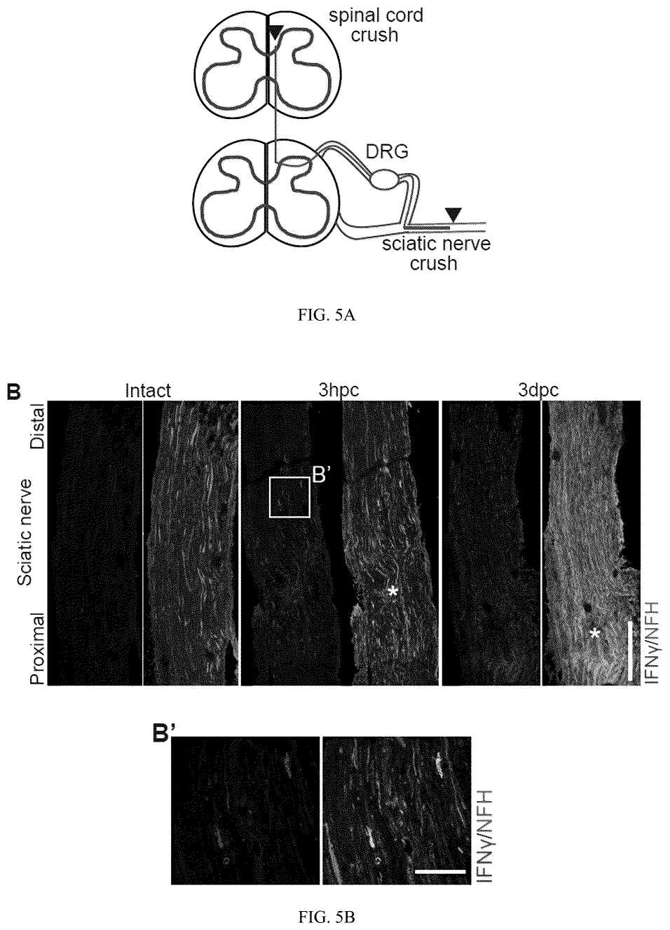

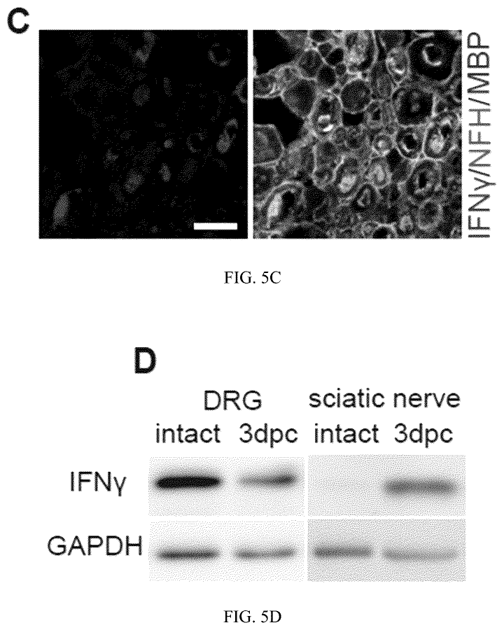

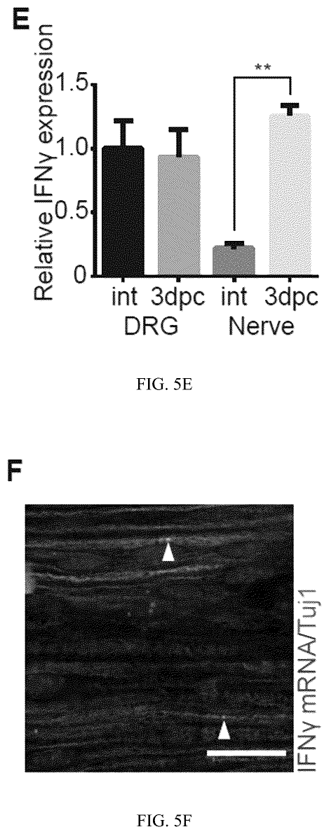

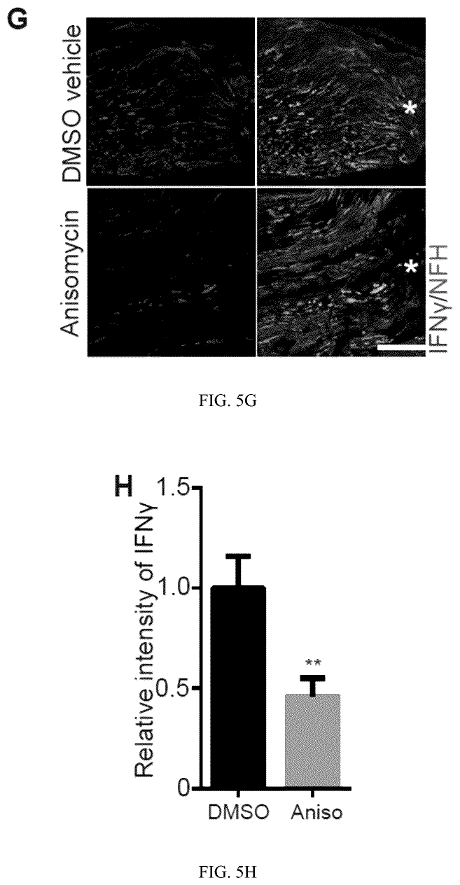

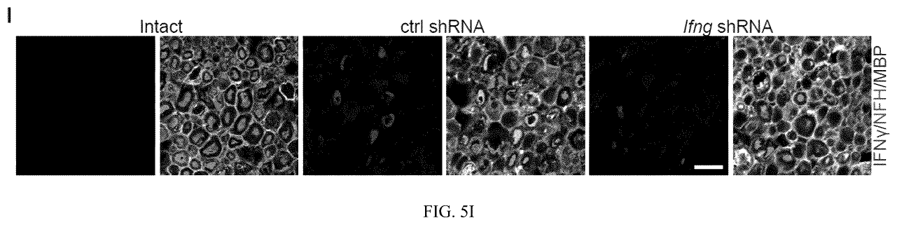

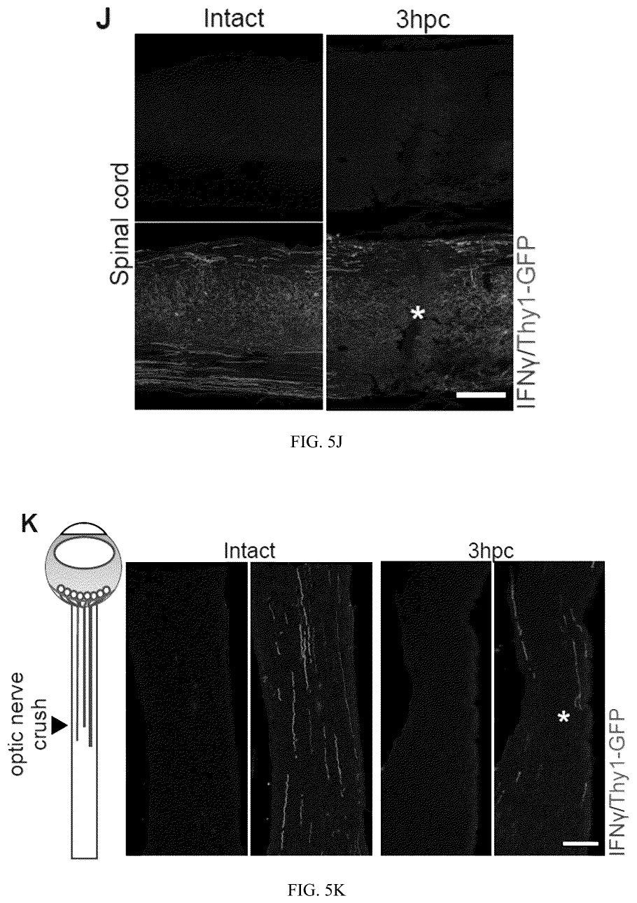

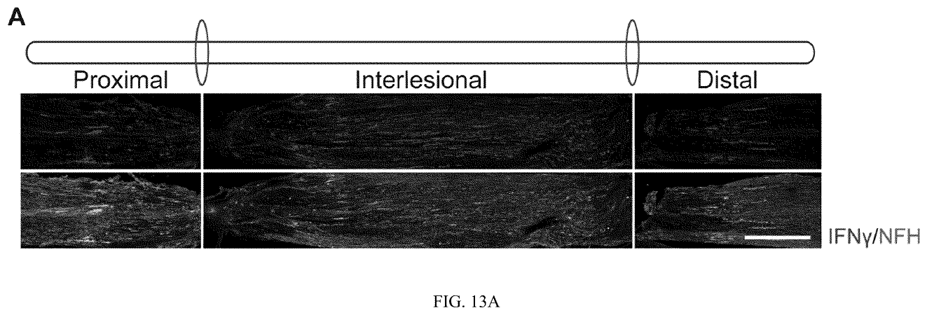

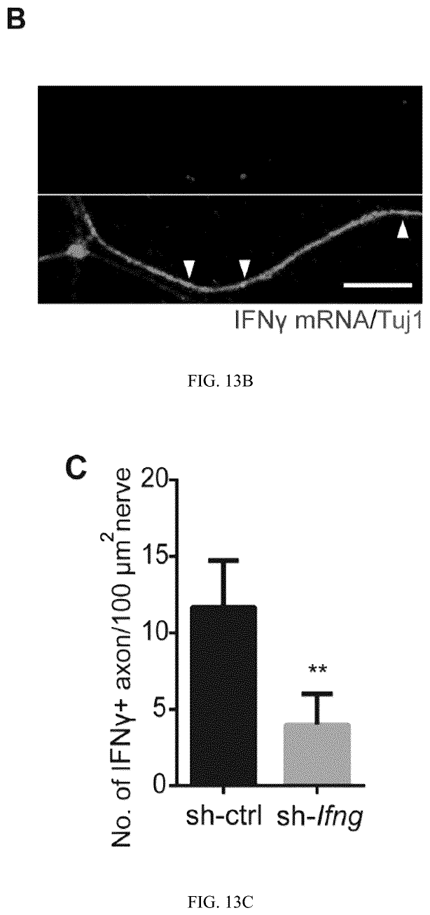

Since axons spontaneously regenerate after injury in the adult peripheral nervous system, we were curious how the IFNγ and cGAS-STING pathways are regulated and whether they play any role in the regeneration process. Three days after sciatic nerve injury in adult WT mice, we examined the level of IFNγ in the DRGs and sciatic nerves (FIG. 5A). We performed immunostaining and found that elevated IFNγ almost exclusively colocalized with axons in the injured sciatic nerve at 3 hours post crush (hpc) and at 3 days post crush (dpc) (FIGS. 5B and 5C). Western blot showed that the level of IFNγ was high in DRGs and not significantly changed after injury, while IFNγ was weakly detected in the uninjured nerve and evidently increased after injury (FIGS. 5D-5E), consistent with immunostaining. Then, we carried out the double-ligation experiment to check whether the IFNγ elevation in the injured axons was due to the axonal transport from either the DRG neuron soma or the axon terminal. We found that IFNγ signal showed up in the proximal ligation site, the interlesional segment, and the distal ligation site, without obvious accumulations at the notches (FIG. 13A), indicating that axonal transport did not play an essential role in increasing the axonal IFNγ.

To examine whether Ifng mRNA was transported to axons before injury, we performed in situ hybridization chain reaction (HCR) (Choi et al., 2016) coupled with Tuj 1 immunostaining to detect Ifng mRNA in cultured DRG neurons. The result indicated that Ifng mRNA colocalized with the DRG neurites (FIG. 13B). Then we further asked if Ifng mRNA was also stored in peripheral axons of DRG neuron in vivo. For in-vivo experiment, we used RNAScope to amplify the signal of Ifng mRNA on sections of sciatic nerves. Consistent with the in-vitro experiment, Ifng mRNA was detected in axons of sciatic nerves (FIG. 5F). To confirm whether the Ifng mRNA was translated in axons, we utilized an ex-vivo model to test the effect of translation inhibition. Sciatic nerve was first crushed and cut into segments. The injured nerve segments were then incubated in DMEM with a translation inhibitor, anisomycin. We then performed IFNγ immunostaining after a 3-hour incubation. Quantification indicated that anisomycin treatment reduced over 50% of the IFNγ signal (FIGS. 5G and 5H). To exclude the possibility that axonal IFNγ is derived from non-neuronal cells within the sciatic nerve, we delivered AAV9-shRNA-Ifng intrathecally to knock down Ifng in DRG neurons. As shown by the immunostaining and quantification of IFNγ levels in the cross sections of the injured sciatic nerves, this knockdown method effectively infected DRG neurons and deceased the axonal level of IFNγ by over 60% (FIGS. 5I and 13C). In dramatic contrast to what we found in the PNS, we did not detect the elevation of axonal IFNγ in either the central spinal branches of the DRG neurons after spinal cord injury (FIGS. 5J and 13D) or in the axons of the optic nerve after injury (FIGS. 5K and 13D), in which Thy1-GFP mice were used to label individual axons. Our results indicate that IFNγ is locally translated within the axons upon peripheral axotomy.

### Example 6—Axonal IFNγ is Required for Peripheral Axon Regeneration

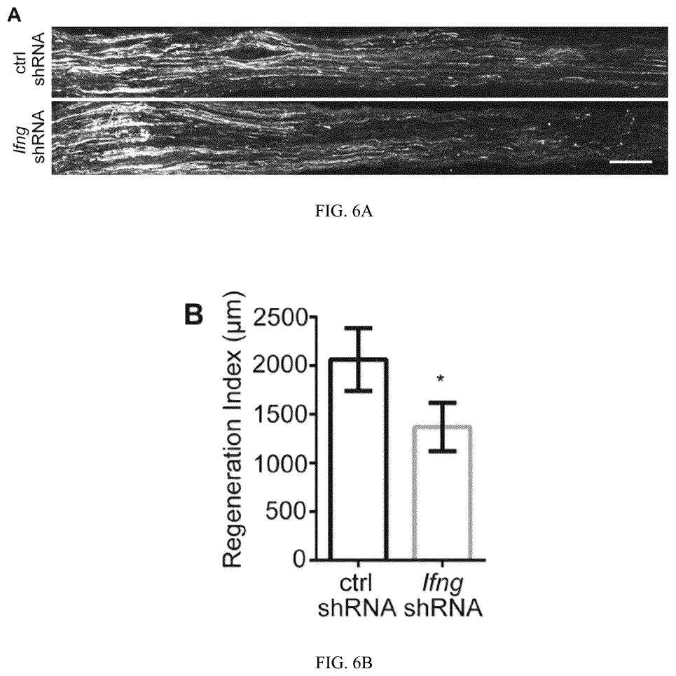

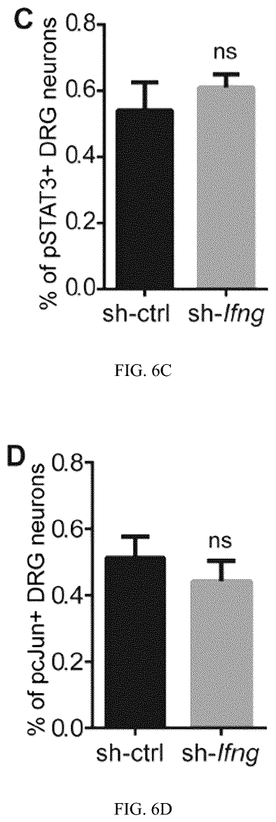

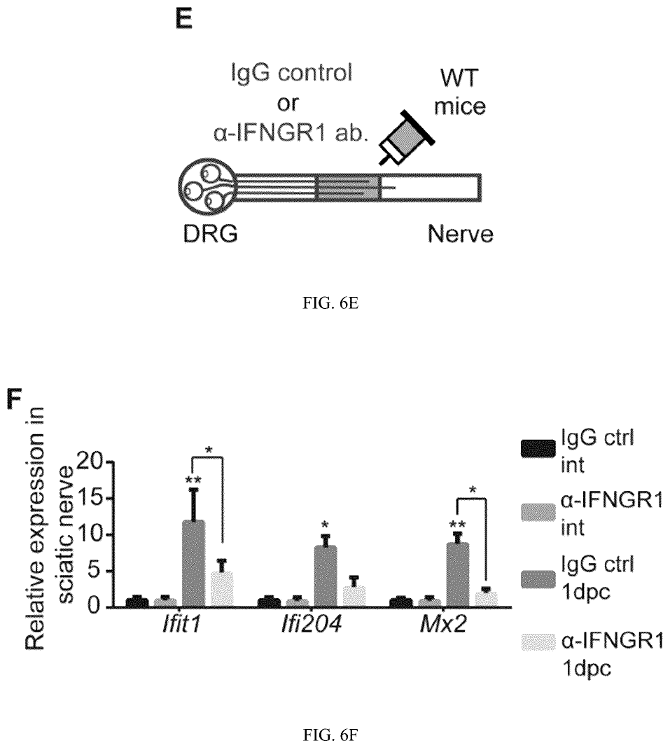

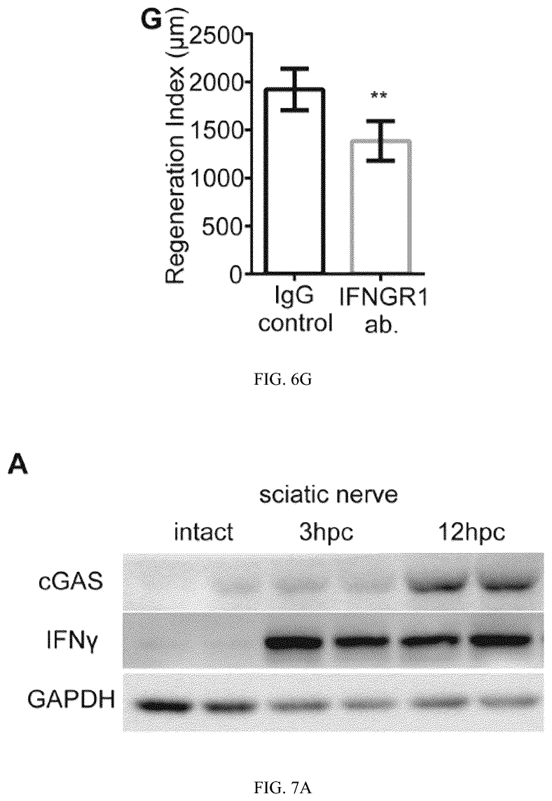

To examine the function of IFNγ in the regeneration, we delivered AAV9-shRNA-Ifng intrathecally to knock down Ifng in DRG neurons. At 3 dpc, we evaluated the sensory axon regeneration by SCG10 staining and observed that AAV-shRNA-Ifng partially suppressed spontaneous axon regeneration in comparison with control shRNA (FIGS. 6A and 6B). Then we investigated the underlying mechanism. Firstly, the elevation of neither p-STAT3 nor phosphorylated cJun (p-cJun) in injured DRG neurons was affected by Ifng knockdown (FIGS. 6C and 6D), indicating that regeneration inhibition was unlikely mediated by targeting canonical retrograde injury signals (Shin et al., 2012). Next, we analyzed a published transcriptomic dataset comparing the DRG response to sciatic nerve axotomy (SNA) and dorsal column axotomy (DCA). The result showed that four ISGs were upregulated at 3 dpc after SNA, but not DCA (FIG. 14A). To examine whether IFNγ signaling regulates axonal regeneration in a cell-autonomous fashion, we deleted Ifngr1 from DRG neurons by intrathecal delivery of AAV9-Cre into Ifngr1 foxed mice (FIG. 14B). The efficacy of AAV was demonstrated using the Tomato Cre reporter mouse line, and over 90% of lumbar DRG neurons were infected by AAV (FIG. 14C). Then, we crushed the sciatic nerve and examined the regeneration. Although Ifngr1 KO decreased the expression of some ISGs (FIG. 14D), we did not observe obvious differences in the axon regeneration between the AAV-Cre and AAV-GFP groups (FIG. 14E and 14F). In the isolated DRG neuron culture, RU.521, a potent and selective inhibitor of cGAS, did not show any effect on the axon elongation, indicating that cGAS in DRG neurons is unlikely to be critical for axon regeneration (FIG. 14G and 14H). Then, we assessed whether IFNγ may influence the regeneration locally at the lesion site by injecting IFNGR1 antibody into the sciatic nerve at the time of injury (FIG. 6E). The efficacy of the antibody was demonstrated by the suppression of some of the signature ISGs in the injured sciatic nerve (FIG. 6F), which also indicates that the released IFNγ from axons triggered the expression of many ISGs in the cells of the injured sciatic nerve. We found that spontaneous regeneration was inhibited by IFNGR1 antibody to a similar degree as by Ifng shRNA, but not by the control antibody (FIG. 6G). Our results support the notion that axonal IFNγ stimulates axon regeneration in a non-cell-autonomous manner after sciatic nerve injury.

### Example 7—IFNγ Elevates cGAS Expression in the Injured Sciatic Nerve to Promote Axon Regeneration

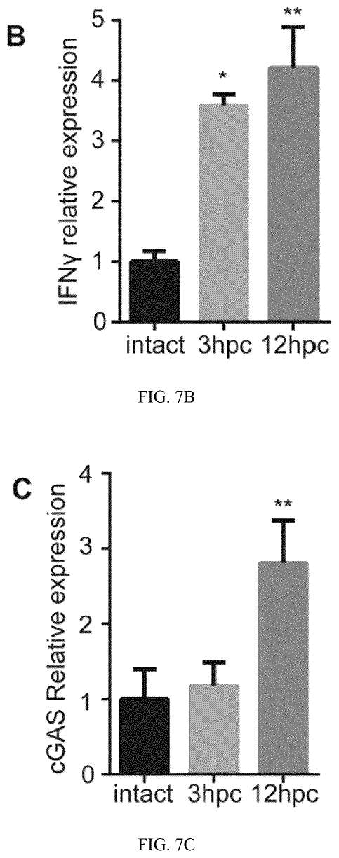

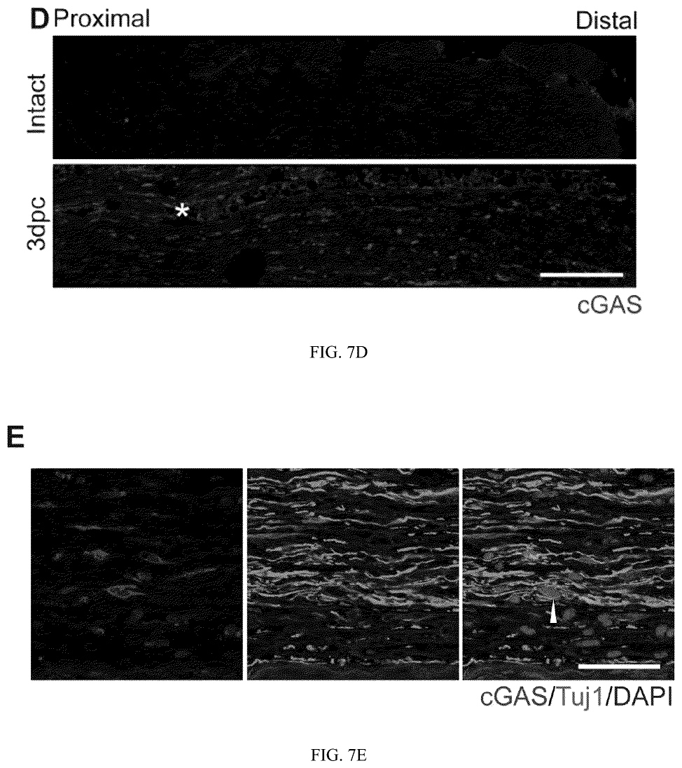

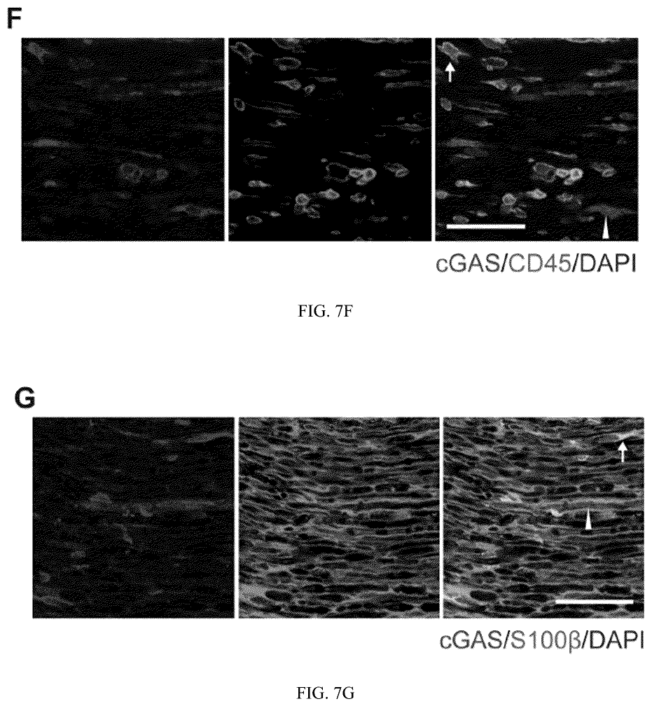

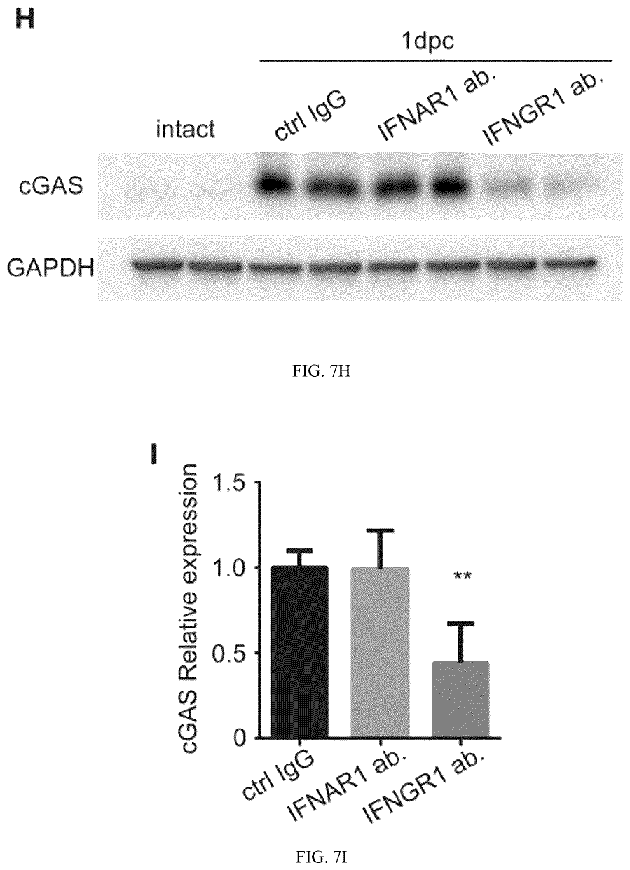

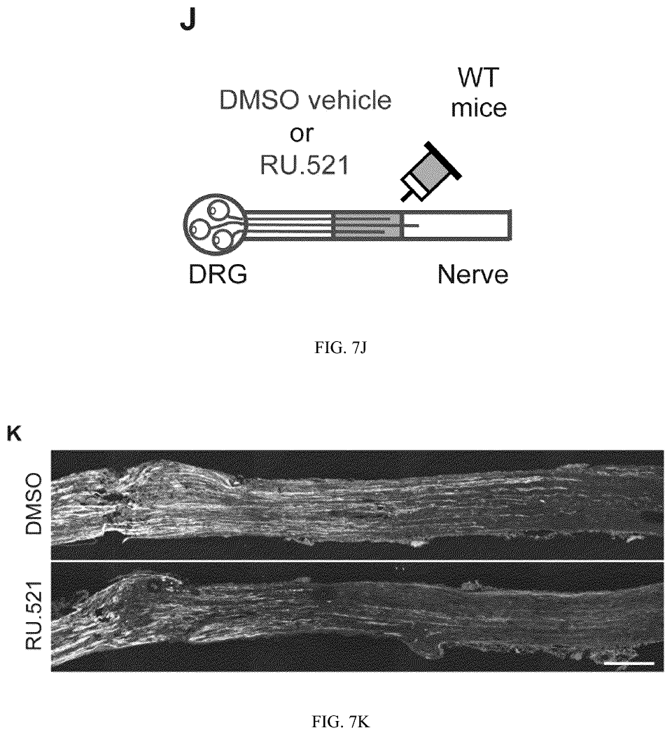

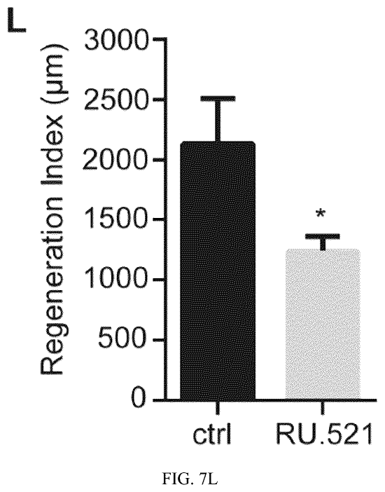

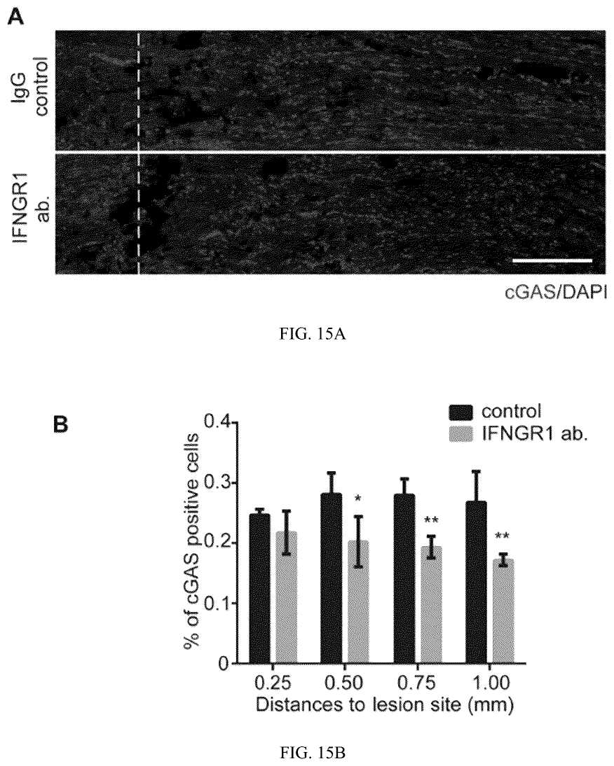

Peripheral axon regeneration is facilitated by many different types of cells in the injured nerves (Cattin and Lloyd, 2016; Kalinski et al., 2020), and we hypothesized that IFNγ may regulate axon regeneration indirectly by stimulating these non-neuronal cells. From our RGC experiments (FIG. 4A), one such candidate molecule is cGAS. By performing western blotting of the injured sciatic nerve protein samples, we found that IFNγ was increased within 3 hours, while cGAS was significantly elevated at 12 hpc (FIGS. 7A-7C). At 3 dpc, we performed immunostaining of the sciatic nerve and found that many cGAS+cells were observed around and distal to the lesion site (FIG. 7D). cGAS did not colocalize with the axonal marker Tuj 1 (FIG. 7E), but with the hematopoietic cell marker CD45 (FIG. 7F) and the Schwann cell marker S100b (FIG. 7G). To test whether IFNγ stimulates cGAS expression, we infused the IFNGR1 antibody into the lesioned nerve. Western blot analysis shows that the cGAS level was evidently suppressed but not affected by either control antibody or IFNAR1 antibody infusion (FIGS. 7H-7I). Quantification also shows that the number of cGAS+cells distal to the lesion site was decreased (FIGS. 15A-15B). Overall, these results indicated that IFNγ may be secreted or released from injured or degenerating axons and likely stimulates cGAS expression in the Schwann cells and/or hematopoietic cells in the sciatic nerve.

Next, we examined the role of the cGAS elevation in axon regeneration. We injected RU.521 into the crushed sciatic nerve to suppress cGAS expression within the nerve (FIG. 7J). RU.521 administration inhibited axon regeneration by over 40% at 3 days post crush (FIGS. 7K and 7L). Thus, cGAS expression in Schwann cells and/or hematopoietic cells stimulated by axonal IFNγ is essential for the axonal regeneration in the PNS.

### Example 8—Injury-Induced cGAMP Promotes Peripheral Regeneration Through Axonal Sting

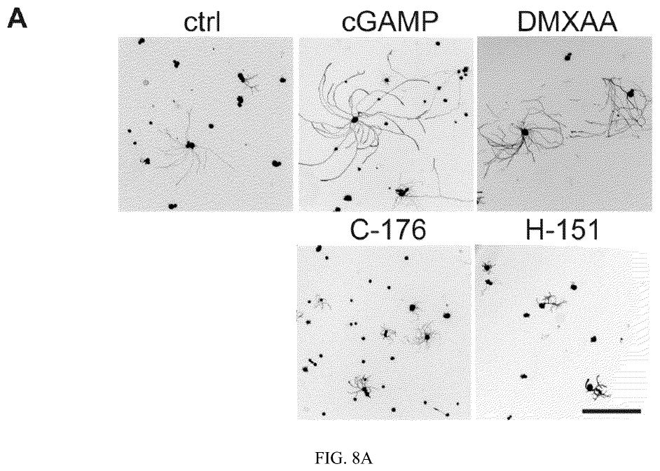

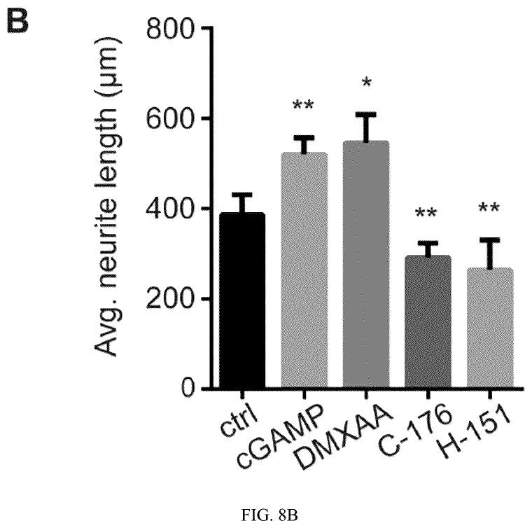

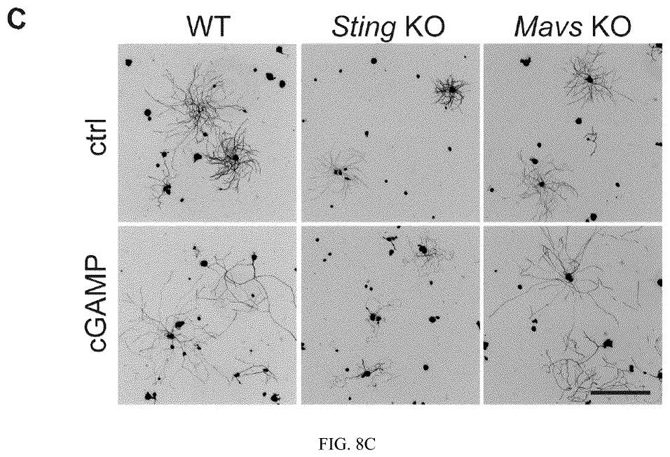

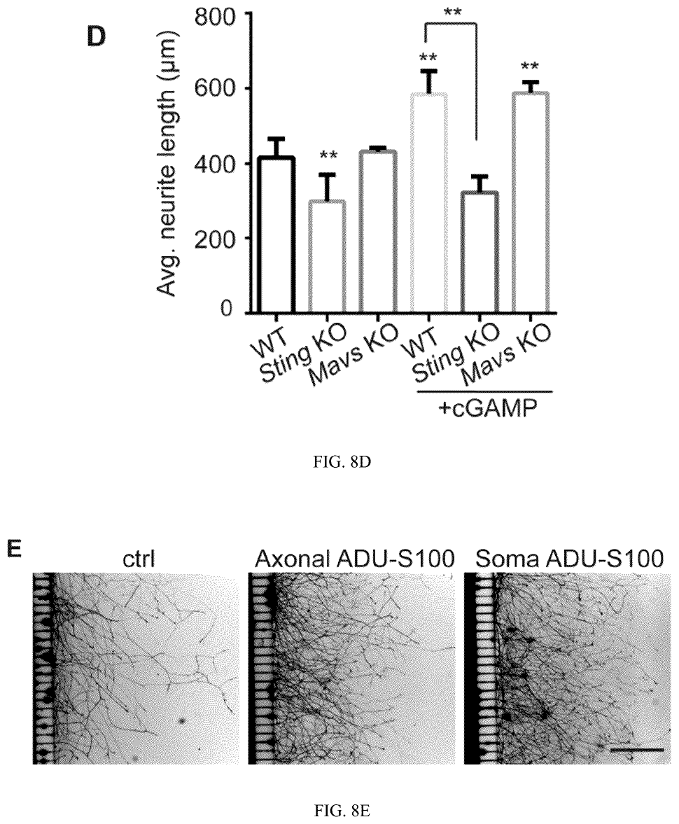

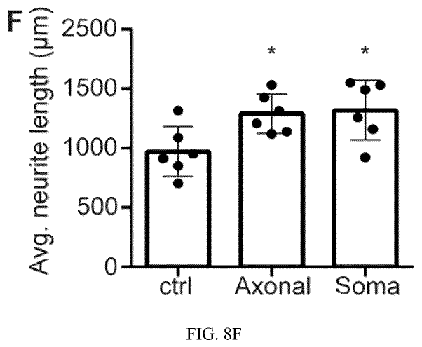

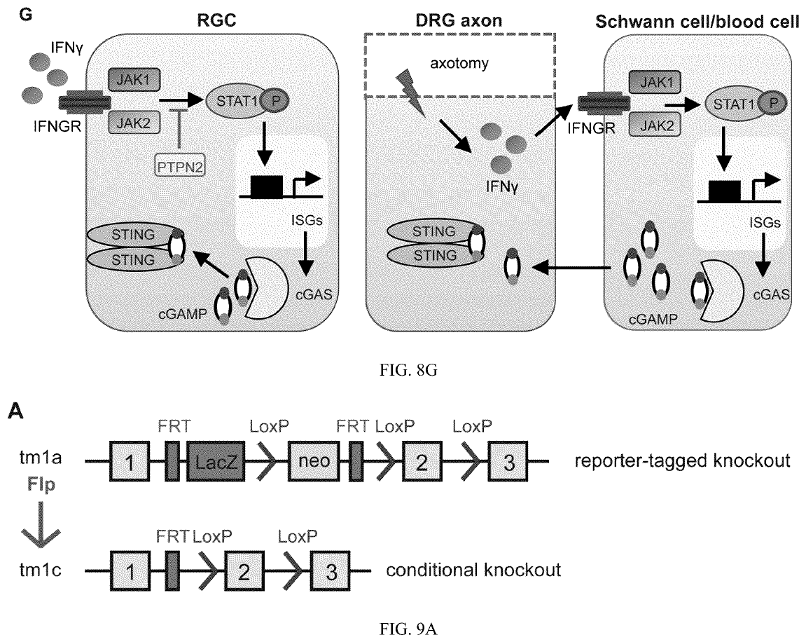

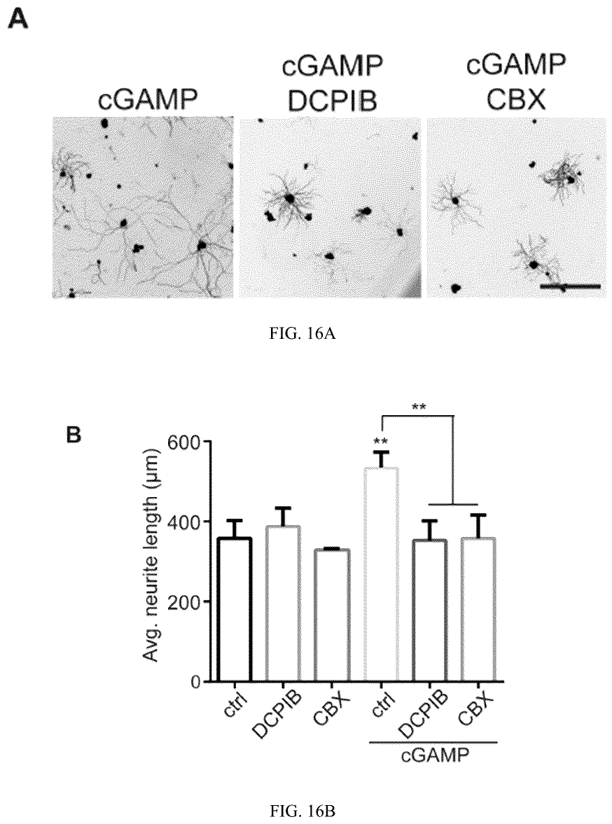

We hypothesized that cGAS elevation in non-neuronal cells within the sciatic nerve may produce cGAMP and enhance axon regeneration by activating STING. In DRG culture, either cGAMP or DMXAA, a mouse-specific STING ligand, enhanced the axon elongation (FIGS. 8A-8B) STING inhibitors decreased the axon growth in vitro (FIGS. 8A-8B). This result was also confirmed by using Sting KO mice (FIGS. 8C-8D). The growth effect of cGAMP on DRG culture was completely abolished with Sting deletion (FIGS. 8C-8D). As a control, Ma vs KO did not show any evident effects with or without cGAMP. As cGAS elevation was mostly found in the lesion site and the distal nerve, this immediately raised the question of how cGAMP works to promote the regrowth. By using microfluidic chambers, we cultured DRG neurons with their cell bodies and axons in separate compartments, where compounds could be added to one compartment without affecting the other. We allowed axons to grow through the channels initially and then severed axons by vacuum aspiration to mimic axotomy. Then, ADU-S100 was added to the axonal compartment or the soma compartment. We found that ADU-S100 treatment on either side enhanced axon elongation (FIGS. 8E-8F). LRRC8 volume-regulated anion channels have been identified as cGAMP transporters (Lahey et al., 2020; Zhou et al., 2020). Adding LRRC8 blocker DCPIB or CBX into DRG culture could inhibit the growth effect induced by cGAMP (FIGS. 16A-16B), indicating that cGAMP is imported into DRG neurons through these channels. Thus, locally produced 2′,3′-cGAMP could enhance the axon regeneration by activating STING in the axons.

### Example 9—Methods of Axon Regeneration

First, in the CNS, the activation of the IFNγ-STAT1 pathway promotes axon regeneration through a neuron-autonomous mechanism. This result suggests that retinas control their immune response by multiple layers of inhibition, and the removal or suppression of such inhibitors (e.g., PTPN2) can assist RGC axon regeneration. Second, in the PNS, IFNγ is secreted by injured axons and promotes axon regeneration through a non-cell-autonomous mechanism by upregulating cGAS expression in Schwann cells and blood cells. Finally, cGAS may produce the immunotransmitter 2′,3′-cGAMP, and neuronal STING senses cGAMP to mediate axon regeneration. Collectively, our study shows that different immune responses after CNS and PNS injury mediate the differential axon regeneration outcomes and elucidates how the antiviral mechanism is coordinated to promote axon regeneration.

Upon virus infection, intracellular pattern recognition receptors (PRRs) sense cytosolic viral DNA or RNA, then trigger the expression of interferons to activate JAK-STAT signaling (Ma et al., 2015a; Ma et al., 2015b). In our study, we found that neuronal cGAS in the CNS or non-neuronal cGAS in the PNS could be upregulated by IFNγ-STAT1 signaling and facilitate axon regeneration. In our study, we discovered that IFNγ is synthesized in axons after axotomy and can modify the niche for axon regeneration. Surprisingly, the production of IFNγ only occurs in the peripheral, but not in the central axons of DRG. Given the vulnerability of CNS neurons, this unilateral enhancement could be a mechanism to prevent the potential detrimental effect of cell death induced by excessive IFNγ, while also restricting the extent of the environmental injury response

In our experiment, we observed the secretion of IFNγ from axons after sciatic nerve injury. Consistent with our results, Song et al. found that treating peripheral axons with IFNγ in the compartmental culture reduced retrograde viral infection of the neuronal cell body (Song et al., 2016). These results suggested that neuroinflammation may be involved in the antipathogen function and axonal regeneration at the same time. After nerve injury, IFNγ stimulated cGAS expression in Schwann cells and blood cells in the sciatic nerve. cGAS produced cGAMP, which might subsequently further increase the level of interferons in the nerve and DRG. Such a feedback loop possibly simultaneously maintains the expression of ISGs to sustain axon regeneration and prevents potential viral spreading. Our findings also raise possible implications for loss of the regeneration capacity in CNS neurons and the evolution of pathogen resistance (Blackshaw, 2022; Wu et al., 2018).

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Ablasser, A., and Chen, Z. J. (2019). cGAS in action: Expanding roles in immunity and inflammation. Science (New York, N.Y 363. 10.1126/science.aat8657.
Baldwin, K. T., Carbajal, K. S., Segal, B. M., and Giger, R. J. (2015). Neuroinflammation triggered by beta-glucan/dectin-1 signaling enables CNS axon regeneration. Proc Natl Acad Sci USA 112, 2581-2586. 10.1073/pnas.1423221112.
Bei, F., Lee, H. H., Liu, X., Gunner, G., Jin, H., Ma, L., Wang, C., Hou, L., Hensch, T. K., Frank, E., et al. (2016). Restoration of Visual Function by Enhancing Conduction in Regenerated Axons. Cell 164, 219-232. 10.1016/j.cell.2015.11.036.
Ben-Yaakov, K., Dagan, S. Y., Segal-Ruder, Y., Shalem, O., Vuppalanchi, D., Willis, D. E., Yudin, D., Rishal, I., Rother, F., Bader, M., et al. (2012). Axonal transcription factors signal retrogradely in lesioned peripheral nerve. EMBO J 31, 1350-1363. 10.1038/emboj.2011.494.
Benowitz, L. I., and Popovich, P. G. (2011). Inflammation and axon regeneration. Curr Opin Neurol 24, 577-583. 10.1097/WCO.0b013e32834c208d.
Blackshaw, S. (2022). Why Has the Ability to Regenerate Following CNS Injury Been Repeatedly Lost Over the Course of Evolution? Front Neurosci 16, 831062. 10.3389/fnins.2022.831062.
Bray, E. R., Yungher, B. J., Levay, K., Ribeiro, M., Dvoryanchikov, G., Ayupe, A. C., Thakor, K., Marks, V., Randolph, M., Danzi, M. C., et al. (2019). Thrombospondin-1 Mediates Axon Regeneration in Retinal Ganglion Cells. Neuron 103, 642-657 e647. 10.1016/j.neuron.2019.05.044.
Cattin, A. L., and Lloyd, A. C. (2016). The multicellular complexity of peripheral nerve regeneration. Current opinion in neurobiology 39, 38-46. 10.1016/j.conb.2016.04.005.
Chandran, V., Coppola, G., Nawabi, H., Omura, T., Versano, R., Huebner, E. A., Zhang, A., Costigan, M., Yekkirala, A., Barrett, L., et al. (2016). A Systems-Level Analysis of the Peripheral Nerve Intrinsic Axonal Growth Program. Neuron 89, 956-970. 10.1016/j.neuron.2016.01.034.
Cheng, Y. C., Snavely, A., Barrett, L. B., Zhang, X., Herman, C., Frost, D. J., Riva, P., Tochitsky, I., Kawaguchi, R., Singh, B., et al. (2021). Topoisomerase I inhibition and peripheral nerve injury induce DNA breaks and ATF3-associated axon regeneration in sensory neurons. Cell Rep 36, 109666. 10.1016/j.celrep.2021.109666.
Choi, H. M., Calvert, C. R., Husain, N., Huss, D., Barsi, J. C., Deverman, B. E., Hunter, R. C., Kato, M., Lee, S. M., Abelin, A. C., et al. (2016). Mapping a multiplexed zoo of mRNA expression. Development 143, 3632-3637. 10.1242/dev.140137.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21. 10.1093/bioinformatics/bts635.
Dodd, G. T., Decherf, S., Loh, K., Simonds, S. E., Wiede, F., Balland, E., Merry, T. L., Munzberg, H., Zhang, Z. Y., Kahn, B. B., et al. (2015). Leptin and insulin act on POMC neurons to promote the browning of white fat. Cell 160, 88-104. 10.1016/j.cell.2014.12.022.
Donnelly, C. R., Jiang, C., Andriessen, A. S., Wang, K., Wang, Z., Ding, H., Zhao, J., Luo, X., Lee, M. S., Lei, Y. L., et al. (2021). STING controls nociception via type I interferon signalling in sensory neurons. Nature 591, 275-280. 10.1038/s41586-020-03151-1.
Duan, X., Qiao, M., Bei, F., Kim, I. J., He, Z., and Sanes, J. R. (2015). Subtype-Specific Regeneration of Retinal Ganglion Cells following Axotomy: Effects of Osteopontin and mTOR Signaling. Neuron 85, 1244-1256. 10.1016/j.neuron.2015.02.017.
Fawcett, J. W. (2020). The Struggle to Make CNS Axons Regenerate: Why Has It Been so Difficult? Neurochem Res 45, 144-158. 10.1007/s11064-019-02844-y.
Feng, G., Mellor, R. H., Bernstein, M., Keller-Peck, C., Nguyen, Q. T., Wallace, M., Nerbonne, J. M., Lichtman, J. W., and Sanes, J. R. (2000). Imaging neuronal subsets in transgenic mice expressing multiple spectral variants of GFP. Neuron 28, 41-51. 10.1016/s0896-6273(00)00084-2.
Filiano, A. J., Xu, Y., Tustison, N. J., Marsh, R. L., Baker, W., Smirnov, I., Overall, C. C., Gadani, S. P., Turner, S. D., Weng, Z., et al. (2016). Unexpected role of interferon-gamma in regulating neuronal connectivity and social behaviour. Nature 535, 425-429. 10.1038/nature18626.
Fisher, D., Xing, B., Dill, J., Li, H., Hoang, H. H., Zhao, Z., Yang, X. L., Bachoo, R., Cannon, S., Longo, F. M., et al. (2011). Leukocyte common antigen-related phosphatase is a functional receptor for chondroitin sulfate proteoglycan axon growth inhibitors. J Neurosci 31, 14051-14066. 10.1523/JNEUROSCI.1737-11.2011.
Frey, E., Valakh, V., Karney-Grobe, S., Shi, Y., Milbrandt, J., and DiAntonio, A. (2015). An in vitro assay to study induction of the regenerative state in sensory neurons. Experimental neurology 263, 350-363. 10.1016/j.expneurol.2014.10.012.
He, Z., and Jin, Y. (2016). Intrinsic Control of Axon Regeneration. Neuron 90, 437-451. 10.1016/j.neuron.2016.04.022.
Hilton, B. J., Husch, A., Schaffran, B., Lin, T. C., Burnside, E. R., Dupraz, S., Schelski, M., Kim, J., Muller, J. A., Schoch, S., et al. (2022). An active vesicle priming machinery suppresses axon regeneration upon adult CNS injury. Neuron 110, 51-69 e57. 10.1016/j.neuron.2021.10.007.
Holt, C. E., Martin, K. C., and Schuman, E. M. (2019). Local translation in neurons: visualization and function. Nat Struct Mol Biol 26, 557-566. 10.1038/s41594-019-0263-5.
Hu, Z., Deng, N., Liu, K., Zhou, N., Sun, Y., and Zeng, W. (2020). CNTF-STAT3-IL-6 Axis Mediates Neuroinflammatory Cascade across Schwann Cell-Neuron-Microglia. Cell Rep 31, 107657. 10.1016/j.celrep.2020.107657.
Hubel, P., Urban, C., Bergant, V., Schneider, W. M., Knauer, B., Stukalov, A., Scaturro, P., Mann, A., Brunotte, L., Hoffmann, H. H., et al. (2019). A protein-interaction network of interferon-stimulated genes extends the innate immune system landscape. Nat Immunol 20, 493-502. 10.1038/s41590-019-0323-3.
Jin, L., Hill, K .K., Filak, H., Mogan, J., Knowles, H., Zhang, B., Perraud, A. L., Cambier, J. C., and Lenz, L. L. (2011). MPYS is required for IFN response factor 3 activation and type I IFN production in the response of cultured phagocytes to bacterial second messengers cyclic-di-AMP and cyclic-di-GMP. J Immunol 187, 2595-2601. 10.4049/jimmunol.1100088.
Kalinski, A. L., Yoon, C., Huffman, L. D., Duncker, P. C., Kohen, R., Passino, R., Hafner, H., Johnson, C., Kawaguchi, R., Carbaj al, K. S., et al. (2020). Analysis of the immune response to sciatic nerve injury identifies efferocytosis as a key mechanism of nerve debridement. Elife 9. 10.7554/eLife.60223.
Kar, A. N., Lee, S. J., and Twiss, J. L. (2018). Expanding Axonal Transcriptome Brings New Functions for Axonally Synthesized Proteins in Health and Disease. Neuroscientist 24, 111-129. 10.1177/1073858417712668.
Katkeviciute, E., Hering, L., Montalban-Arques, A., Busenhart, P., Schwarzfischer, M., Manzini, R., Conde, J., Atrott, K., Lang, S., Rogler, G., et al. (2021). Protein tyrosine phosphatase nonreceptor type 2 controls colorectal cancer development. J Clin Invest 131. 10.1172/JCI140281.
Kong, G., Zhou, L., Serger, E., Palmisano, I., De Virgiliis, F., Hutson, T. H., McLachlan, E., Freiwald, A., La Montanara, P., Shkura, K., et al. (2020). AMPK controls the axonal regenerative ability of dorsal root ganglia sensory neurons after spinal cord injury. Nat Metab 2, 918-933. 10.1038/s42255-020-0252-3.
LaFleur, M. W., Nguyen, T. H., Coxe, M. A., Miller, B. C., Yates, K. B., Gillis, J. E., Sen, D. R., Gaudiano, E. F., Al Abosy, R., Freeman, G. J., et al. (2019). PTPN2 regulates the generation of exhausted CD8(+) T cell subpopulations and restrains tumor immunity. Nat Immunol 20, 1335-1347. 10.1038/s41590-019-0480-4.
Lahey, L. J., Mardjuki, R. E., Wen, X., Hess, G. T., Ritchie, C., Carozza, J. A., Böhnert, V., Maduke, M., Bassik, M. C., and Li, L. (2020). LRRC8A: C/E heteromeric channels are ubiquitous transporters of cGAMP. Molecular Cell 80, 578-591. e575.
Lau, J., Minett, M. S., Zhao, J., Dennehy, U., Wang, F., Wood, J. N., and Bogdanov, Y. D. (2011). Temporal control of gene deletion in sensory ganglia using a tamoxifen-inducible Advillin-Cre-ERT2 recombinase mouse. Mol Pain 7, 100. 10.1186/1744-8069-7-100.
Lee, S. H., Carrero, J. A., Uppaluri, R., White, J. M., Archambault, J. M., Lai, K. S., Chan, S. R., Sheehan, K. C., Unanue, E. R., and Schreiber, R. D. (2013). Identifying the initiating events of anti-Listeria responses using mice with conditional loss of IFN-gamma receptor subunit 1 (IFNGR1). J Immunol 191, 4223-4234. 10.4049/jimmunol.1300910.
Li, S., He, Q., Wang, H., Tang, X., Ho, K. W., Gao, X., Zhang, Q., Shen, Y., Cheung, A., Wong, F., et al. (2015). Injured adult retinal axons with Pten and Socs3 co-deletion reform active synapses with suprachiasmatic neurons. Neurobiol Dis 73, 366-376. 10.1016/j.nbd.2014.09.019.
Liu, H., Zhang, H., Wu, X., Ma, D., Wu, J., Wang, L., Jiang, Y., Fei, Y., Zhu, C., Tan, R., et al. (2018). Nuclear cGAS suppresses DNA repair and promotes tumorigenesis. Nature 563, 131-136. 10.1038/s41586-018-0629-6.
Liu, K., Lu, Y., Lee, J. K., Samara, R., Willenberg, R., Sears-Kraxberger, I., Tedeschi, A., Park, K. K., Jin, D., Cai, B., et al. (2010). PTEN deletion enhances the regenerative ability of adult corticospinal neurons. Nat Neurosci 13, 1075-1081. nn.2603 [pii] 10.1038/nn.2603.
Love, M. I., Huber, W., and Anders, S. (2014). Moderated estimation of fold change and dispersion for RNA-seq data with DESeq2. Genome Biol 15, 550. 10.1186/s13059-014-0550-8.
Ma, F., Li, B., Liu, S. Y., Iyer, S. S., Yu, Y., Wu, A., and Cheng, G. (2015a). Positive feedback regulation of type I IFN production by the IFN-inducible DNA sensor cGAS. J Immunol 194, 1545-1554. 10.4049/jimmunol.1402066.
Ma, F., Li, B., Yu, Y., Iyer, S. S., Sun, M., and Cheng, G. (2015b). Positive feedback regulation of type I interferon by the interferon-stimulated gene STING. EMBO Rep 16, 202-212. 10.15252/embr.201439366.
Madisen, L., Zwingman, T. A., Sunkin, S. M., Oh, S. W., Zariwala, H. A., Gu, H., Ng, L. L., Palmiter, R. D., Hawrylycz, M. J., Jones, A. R., et al. (2010). A robust and high-throughput Cre reporting and characterization system for the whole mouse brain. Nat Neurosci 13, 133-140. 10.1038/nn.2467.
Mahar, M., and Cavalli, V. (2018). Intrinsic mechanisms of neuronal axon regeneration. Nat Rev Neurosci 19, 323-337. 10.1038/s41583-018-0001-8.
Manguso, R. T., Pope, H. W., Zimmer, M. D., Brown, F. D., Yates, K. B., Miller, B. C., Collins, N. B., Bi, K., LaFleur, M. W., Juneja, V. R., et al. (2017). In vivo CRISPR screening identifies Ptpn2 as a cancer immunotherapy target. Nature 547, 413-418. 10.1038/nature23270.
Moh, A., Iwamoto, Y., Chai, G. X., Zhang, S. S., Kano, A., Yang, D. D., Zhang, W., Wang, J., Jacoby, J. J., Gao, B., et al. (2007). Role of STAT3 in liver regeneration: survival, DNA synthesis, inflammatory reaction and liver mass recovery. Lab Invest 87, 1018-1028. 10.1038/labinvest.3700630.
Neumann, S., and Woolf, C. J. (1999). Regeneration of dorsal column fibers into and beyond the lesion site following adult spinal cord injury. Neuron 23, 83-91.
Palmisano, I., Danzi, M. C., Hutson, T. H., Zhou, L., McLachlan, E., Serger, E., Shkura, K., Srivastava, P. K., Hervera, A., Neill, N. O., et al. (2019). Epigenomic signatures underpin the axonal regenerative ability of dorsal root ganglia sensory neurons. Nat Neurosci 22, 1913-1924. 10.1038/s41593-019-0490-4.
Park, K. K., Liu, K., Hu, Y., Smith, P. D., Wang, C., Cai, B., Xu, B., Connolly, L., Kramvis, I., Sahin, M., and He, Z. (2008). Promoting axon regeneration in the adult CNS by modulation of the PTEN/mTOR pathway. Science (New York, N.Y 322, 963-966.
Picelli, S., Faridani, O. R., Bjorklund, A. K., Winberg, G., Sagasser, S., and Sandberg, R. (2014). Full-length RNA-seq from single cells using Smart-seq2. Nat Protoc 9, 171-181. 10.1038/nprot.2014.006.
Platt, R. J., Chen, S., Zhou, Y., Yim, M. J., Swiech, L., Kempton, H. R., Dahlman, J. E., Parnas, O., Eisenhaure, T. M., Jovanovic, M., et al. (2014). CRISPR-Cas9 knockin mice for genome editing and cancer modeling. Cell 159, 440-455. 10.1016/j.cell.2014.09.014.
Qiu, J., Cafferty, W. B., McMahon, S. B., and Thompson, S. W. (2005). Conditioning injury-induced spinal axon regeneration requires signal transducer and activator of transcription 3 activation. J Neurosci 25, 1645-1653. 25/7/1645 [pii] 10.1523/JNEUROSCI.3269-04.2005.
Risson, V., Mazelin, L., Roceri, M., Sanchez, H., Moncollin, V., Corneloup, C., Richard-Bulteau, H., Vignaud, A., Baas, D., Defour, A., et al. (2009). Muscle inactivation of mTOR causes metabolic and dystrophin defects leading to severe myopathy. J Cell Biol 187, 859-874. 10.1083/jcb.200903131.
Saijilafu, Hur, E. M., Liu, C. M., Jiao, Z., Xu, W. L., and Zhou, F. Q. (2013). PI3K-GSK3 signalling regulates mammalian axon regeneration by inducing the expression of Smad1. Nat Commun 4, 2690. 10.1038/ncomms3690.
Sas, A. R., Carbajal, K. S., Jerome, A. D., Menon, R., Yoon, C., Kalinski, A. L., Giger, R. J., and Segal, B. M. (2020). A new neutrophil subset promotes CNS neuron survival and axon regeneration. Nat Immunol 21, 1496-1505. 10.1038/s41590-020-00813-0.
Schneider, W. M., Chevillotte, M. D., and Rice, C. M. (2014). Interferon-stimulated genes: a complex web of host defenses. Annu Rev Immunol 32, 513-545. 10.1146/annurev-immunol-032713-120231.
Schoggins, J. W. (2019). Interferon-Stimulated Genes: What Do They All Do? Annu Rev Virol 6, 567-584. 10.1146/annurev-virology-092818-015756.
Schoggins, J. W., MacDuff, D. A., Imanaka, N., Gainey, M. D., Shrestha, B., Eitson, J. L., Mar, K. B., Richardson, R. B., Ratushny, A. V., Litvak, V., et al. (2014). Pan-viral specificity of IFN-induced genes reveals new roles for cGAS in innate immunity. Nature 505, 691-695. 10.1038/nature12862.
Schroder, K., Hertzog, P. J., Ravasi, T., and Hume, D. A. (2004). Interferon-gamma: an overview of signals, mechanisms and functions. J Leukoc Biol 75, 163-189. 10.1189/jlb.0603252.
Shen, Y., Tenney, A. P., Busch, S. A., Horn, K. P., Cuascut, F. X., Liu, K., He, Z., Silver, J., and Flanagan, J. G. (2009). PTPsigma is a receptor for chondroitin sulfate proteoglycan, an inhibitor of neural regeneration. Science (New York, N.Y 326, 592-596. 1178310 [pii] 10.1126/science.1178310.
Shin, J. E., Cho, Y., Beirowski, B., Milbrandt, J., Cavalli, V., and DiAntonio, A. (2012). Dual leucine zipper kinase is required for retrograde injury signaling and axonal regeneration. Neuron 74, 1015-1022. 10.1016/j.neuron.2012.04.028.
Silver, J., Schwab, M. E., and Popovich, P. G. (2014). Central nervous system regenerative failure: role of oligodendrocytes, astrocytes, and microglia. Cold Spring Harb Perspect Biol 7, a020602. 10.1101/cshperspect.a020602.
Smith, P. D., Sun, F., Park, K. K., Cai, B., Wang, C., Kuwako, K., Martinez-Carrasco, I., Connolly, L., and He, Z. (2009). SOCS3 deletion promotes optic nerve regeneration in vivo. Neuron 64, 617-623. S0896-6273(09)00937-4 [pii] 10.1016/j.neuron.2009.11.021.
Song, R., Koyuncu, O. O., Greco, T. M., Diner, B. A., Cristea, I. M., and Enquist, L. W. (2016). Two Modes of the Axonal Interferon Response Limit Alphaherpesvirus Neuroinvasion. mBio 7, e02145-02115. 10.1128/mBio.02145-15.
Sun, F., Park, K. K., Belin, S., Wang, D., Lu, T., Chen, G., Zhang, K., Yeung, C., Feng, G., Yankner, B. A., and He, Z. (2011). Sustained axon regeneration induced by co-deletion of PTEN and SOCS3. Nature 480, 372-375. nature10594 [pii] 10.1038/nature10594.
Sun, Q., Sun, L., Liu, H. H., Chen, X., Seth, R. B., Forman, J., and Chen, Z. J. (2006). The specific and essential role of MAVS in antiviral innate immune responses. Immunity 24, 633-642. 10.1016/j.immuni.2006.04.004.
Swiech, L., Heidenreich, M., Banerjee, A., Habib, N., Li, Y., Trombetta, J., Sur, M., and Zhang, F. (2015). In vivo interrogation of gene function in the mammalian brain using CRISPR-Cas9. Nat Biotechnol 33, 102-106. 10.1038/nbt.3055.
Tedeschi, A., and Bradke, F. (2017). Spatial and temporal arrangement of neuronal intrinsic and extrinsic mechanisms controlling axon regeneration. Current opinion in neurobiology 42, 118-127. 10.1016/j.conb.2016.12.005.
Tedeschi, A., Dupraz, S., Laskowski, C. J., Xue, J., Ulas, T., Beyer, M., Schultze, J. L., and Bradke, F. (2016). The Calcium Channel Subunit Alpha2delta2 Suppresses Axon Regeneration in the Adult CNS. Neuron 92, 419-434. 10.1016/j.neuron.2016.09.026.
Terenzio, M., Schiavo, G., and Fainzilber, M. (2017). Compartmentalized Signaling in Neurons: From Cell Biology to Neuroscience. Neuron 96, 667-679. 10.1016/j.neuron.2017.10.015.
Tran, N. M., Shekhar, K., Whitney, I. E., Jacobi, A., Benhar, I., Hong, G., Yan, W., Adiconis, X., Arnold, M. E., Lee, J. M., et al. (2019). Single-Cell Profiles of Retinal Ganglion Cells Differing in Resilience to Injury Reveal Neuroprotective Genes. Neuron 104, 1039-1055 e1012. 10.1016/j.neuron.2019.11.006.
Tsarouchas, T. M., Wehner, D., Cavone, L., Munir, T., Keatinge, M., Lambertus, M., Underhill, A., Barrett, T., Kassapis, E., Ogryzko, N., et al. (2018). Dynamic control of proinflammatory cytokines Il-1beta and Tnf-alpha by macrophages in zebrafish spinal cord regeneration. Nat Commun 9, 4670. 10.1038/s41467-018-07036-w.
Vong, L., Ye, C., Yang, Z., Choi, B., Chua, S., Jr., and Lowell, B. B. (2011). Leptin action on GABAergic neurons prevents obesity and reduces inhibitory tone to POMC neurons. Neuron 71, 142-154. 10.1016/j.neuron.2011.05.028.
Wang, Q., Zhang, S., Liu, T., Wang, H., Liu, K., Wang, Q., and Zeng, W. (2018). Sarm1/Myd88-5 Regulates Neuronal Intrinsic Immune Response to Traumatic Axonal Injuries. Cell Rep 23, 716-724. 10.1016/j.celrep.2018.03.071.
Warre-Cornish, K., Perfect, L., Nagy, R., Duarte, R. R. R., Reid, M. J., Raval, P., Mueller, A., Evans, A. L., Couch, A., Ghevaert, C., et al. (2020). Interferon-gamma signaling in human iPSC-derived neurons recapitulates neurodevelopmental disorder phenotypes. Sci Adv 6, eaay9506. 10.1126/sciadv.aay9506.
Weng, Y. L., Wang, X., An, R., Cassin, J., Vissers, C., Liu, Y., Liu, Y., Xu, T., Wang, X., Wong, S. Z. H., et al. (2018). Epitranscriptomic m(6)A Regulation of Axon Regeneration in the Adult Mammalian Nervous System. Neuron 97, 313-325 e316. 10.1016/j.neuron.2017.12.036.
Wiede, F., Lu, K. H., Du, X., Liang, S., Hochheiser, K., Dodd, G. T., Goh, P. K., Kearney, C., Meyran, D., Beavis, P. A., et al. (2020). PTPN2 phosphatase deletion in T cells promotes anti-tumour immunity and CAR T-cell efficacy in solid tumours. EMBO J 39, e103637. 10.15252/embj.2019103637.
Wu, X., Dao Thi, V. L., Huang, Y., Billerbeck, E., Saha, D., Hoffmann, H. H., Wang, Y., Silva, L. A. V., Sarbanes, S., Sun, T., et al. (2018). Intrinsic Immunity Shapes Viral Resistance of Stem Cells. Cell 172, 423-438 e425. 10.1016/j.cell.2017.11.018.
Xia, T., Yi, X.-M., Wu, X., Shang, J., and Shu, H.-B. (2019). PTPN1/2-mediated dephosphorylation of MITA/STING promotes its 20S proteasomal degradation and attenuates innate antiviral response. Proceedings of the National Academy of Sciences 116, 20063-20069.
Yang, C., Wang, X., Wang, J., Wang, X., Chen, W., Lu, N., Siniossoglou, S., Yao, Z., and Liu, K. (2020). Rewiring Neuronal Glycerolipid Metabolism Determines the Extent of Axon Regeneration. Neuron 105, 276-292 e275. 10.1016/j.neuron.2019.10.009.
Yasuda, M., Nagappan-Chettiar, S., Johnson-Venkatesh, E. M., and Umemori, H. (2021). An activity-dependent determinant of synapse elimination in the mammalian brain. Neuron 109, 1333-1349 e1336. 10.1016/j.neuron.2021.03.006.
Yin, Y., Cui, Q., Li, Y., Irwin, N., Fischer, D., Harvey, A. R., and Benowitz, L. I. (2003). Macrophage-derived factors stimulate optic nerve regeneration. J Neurosci 23, 2284-2293.
Zhang, D., Liu, C., Li, H., and Jiao, J. (2020). Deficiency of STING Signaling in Embryonic Cerebral Cortex Leads to Neurogenic Abnormalities and Autistic-Like Behaviors. Adv Sci (Weinh) 7, 2002117. 10.1002/advs.202002117.
Zhang, S., Chen, L., Luo, Y., Gunawan, A., Lawrence, D. S., and Zhang, Z. Y. (2009). Acquisition of a potent and selective TC-PTP inhibitor via a stepwise fluorophore-tagged combinatorial synthesis and screening strategy. J Am Chem Soc 131, 13072-13079. 10.1021/ja903733z.
Zhang, Z. Y. (2017). Drugging the Undruggable: Therapeutic Potential of Targeting Protein Tyrosine Phosphatases. Acc Chem Res 50, 122-129. 10.1021/acs.accounts.6b00537.
Zhang, Z. Y., Dodd, G. T., and Tiganis, T. (2015). Protein Tyrosine Phosphatases in Hypothalamic Insulin and Leptin Signaling. Trends Pharmacol Sci 36, 661-674. 10.1016/j.tips.2015.07.003.
Zhou, C., Chen, X., Planells-Cases, R., Chu, J., Wang, L., Cao, L., Li, Z., Lopez-Cayuqueo, K. I., Xie, Y., and Ye, S. (2020). Transfer of cGAMP into bystander cells via LRRC8 volume-regulated anion channels augments STING-mediated interferon responses and anti-viral immunity. Immunity 52, 767-781. e766.

We claim:

1. A method for activating STAT1 signaling, the cGAS-STING pathway, or a combination thereof in a subject with central nervous system (CNS) or peripheral nervous system (PNS) injury, the method comprising:
a) administering IFNγ and a Protein Tyrosine Phosphatase Non-Receptor Type 2 (PTPN2) inhibitor to the subject; administering a STING agonist to the subject; or administering a combination thereof to the subject; or
b) inhibiting the expression of PTPN2 in the subject and administering IFNγ to the subject; administering a STING agonist to the subject; or doing a combination thereof.

2. The method of claim 1, wherein the PTPN2 is inhibited using a short hairpin RNA (shRNA) targeting Ptpn2 or a PTPN2 inhibitor, wherein the PTPN2 inhibitor is compound 8 (Formula (I)), compound-182 (Formula (II)), ABBV-CLS-434 (Formula (III)), or any combination thereof:

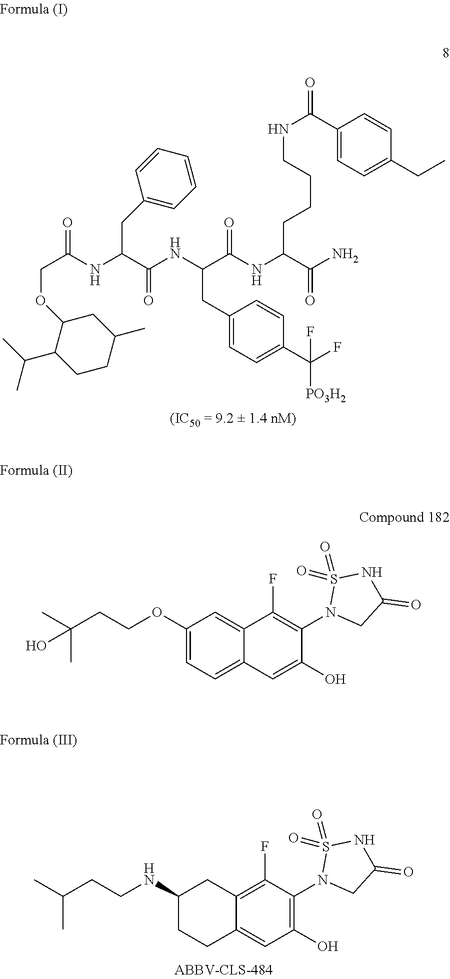

3. The method of claim 2, wherein the concentration of the PTPN2 inhibitor is about 5 μM to about 10 μM.

4. The method of claim 1, wherein the STING agonist is 2′,3′-cGAMP, ADU-S100, 3′,3′-cGAMP, 5,6-Dimethylxanthenone-4-acetic acid (DMXAA), Cridanimod (10-carboxymethyl-9-acridanone), 4-(5,6-dimethoxy-1-benzothiophen-2-yl)-4-oxobutanoic acid (MSA 2), or any combination thereof.

5. The method of claim 1, wherein the concentration of the STING agonist is about 1 mM to about 25 mM.

6. The method of claim 1, further comprising activating STAT3 signaling and mTOR in the subject.

7. The method of claim 1, wherein the activation of STAT1 signaling, the cGAS-STING pathway, or a combination thereof enhances axon regeneration.

8. The method of claim 5, wherein axon regeneration comprises neural repair.

9. The method of claim 5, wherein axon regeneration occurs in injured axons.

10. The method of claim 1, wherein the shRNA is GACAGAGAAATGGTGTTTAA (SEQ ID NO: 21).

11. The method of claim 1, wherein the CNS injury is a spinal cord injury, a traumatic brain injury, or a combination thereof or is caused by a stroke, glaucoma, violent blow, or jolt to the head or body of the subject, or any combination thereof.

12. The method of claim 1, wherein the inhibiting the expression of PTPN2 further comprising inhibiting the expression of Pten, Socs3, or a combination thereof.

13. The method of claim 1, wherein the inhibiting the expression of PTPN2 comprises administering a CRISPR-Cas9 enzyme and a sgRNA targeting PTPN2 to the subject.

14. The method of claim 1, wherein the IFNγ, the PTPN2 inhibitor, the STING agonist, or any combination thereof are administered by intracranial or intravitreous injection.

15. The method of claim 13, wherein the sgRNA is GAACCATCGAGCGGGAGTTCG (SEQ ID NO: 19) or GCCATGTCGGCAACCATCGAG (SEQ ID NO: 20).


SEQUENCE LISTING (37 sequences)

SEQ ID NO: 1 - DNA, 21 bp, synthetic construct
  source 1..21: mol_type = other DNA
gttctgctctgctgaaaaccc

SEQ ID NO: 2 - DNA, 20 bp, synthetic construct
  source 1..20: mol_type = other DNA
cctggtcaccatcagcattc

SEQ ID NO: 3 - DNA, 20 bp, synthetic construct
  source 1..20: mol_type = other DNA
ttccactgaagatgggtggc

SEQ ID NO: 4 - DNA, 20 bp, synthetic construct
  source 1..20: mol_type = other DNA
tctgggttgagtggctttcc

SEQ ID NO: 5 - DNA, 22 bp, synthetic construct
  source 1..22: mol_type = other DNA
accagagtgcaagtgaggagct

SEQ ID NO: 6 - DNA, 22 bp, synthetic construct
  source 1..22: mol_type = other DNA
gtactagggcagtgatgtcctg

SEQ ID NO: 7 - DNA, 20 bp, synthetic construct
  source 1..20: mol_type = other DNA
gcctgaagacctgatcacca

SEQ ID NO: 8 - DNA, 20 bp, synthetic construct
  source 1..20: mol_type = other DNA
ggtagcacaaggcaatggga

SEQ ID NO: 9 - DNA, 20 bp, synthetic construct
  source 1..20: mol_type = other DNA
ctgcagagcagaagaccgaa

SEQ ID NO: 10 - DNA, 20 bp, synthetic construct
  source 1..20: mol_type = other DNA
gggtctacgttgagcagctt

SEQ ID NO: 11 - DNA, 20 bp, synthetic construct
  source 1..20: mol_type = other DNA
aatggtgccctggtctaagc

SEQ ID NO: 12 - DNA, 20 bp, synthetic construct
  source 1..20: mol_type = other DNA
ctggtctgctgggactgttc

SEQ ID NO: 13 - DNA, 20 bp, synthetic construct
  source 1..20: mol_type = other DNA
ggtgtggcatgcctgactat

SEQ ID NO: 14 - DNA, 22 bp, synthetic construct
  source 1..22: mol_type = other DNA
gctcactgtcagtttctctcag

SEQ ID NO: 15 - DNA, 20 bp, synthetic construct
  source 1..20: mol_type = other DNA
tgacaatgccacatacgcca

SEQ ID NO: 16 - DNA, 20 bp, synthetic construct
  source 1..20: mol_type = other DNA
ttcttccgagatggagttgc

SEQ ID NO: 17 - DNA, 19 bp, synthetic construct
  source 1..19: mol_type = other DNA
aactgaccaggcacagacg

SEQ ID NO: 18 - DNA, 20 bp, synthetic construct
  source 1..20: mol_type = other DNA
ccctaatgtcttccgcctgc

SEQ ID NO: 19 - DNA, 21 bp, synthetic construct
  source 1..21: mol_type = other DNA
gaaccatcgagcgggagttcg

SEQ ID NO: 20 - DNA, 21 bp, synthetic construct
  source 1..21: mol_type = other DNA
gccatgtcggcaaccatcgag

SEQ ID NO: 21 - DNA, 20 bp, synthetic construct
  source 1..20: mol_type = other DNA
gacagagaaatggtgtttaa

SEQ ID NO: 22 - DNA, 19 bp, synthetic construct
  source 1..19: mol_type = other DNA
gaccatcaatatgactaga

SEQ ID NO: 23 - DNA, 21 bp, synthetic construct
  source 1..21: mol_type = other DNA
tgtgacaccttgcttgtttat

SEQ ID NO: 24 - DNA, 21 bp, synthetic construct
  source 1..21: mol_type = other DNA
ccagatgaaccttgcactata

SEQ ID NO: 25 - DNA, 21 bp, synthetic construct
  source 1..21: mol_type = other DNA
agctctccgtcctcgtatttc

SEQ ID NO: 26 - DNA, 21 bp, synthetic construct
  source 1..21: mol_type = other DNA
gggactccgtgtcaattattt

SEQ ID NO: 27 - DNA, 20 bp, synthetic construct
  source 1..20: mol_type = other DNA
cgcctttgggaagtattatt

SEQ ID NO: 28 - DNA, 21 bp, synthetic construct
  source 1..21: mol_type = other DNA
gacttagacttgacctatatt

SEQ ID NO: 29 - DNA, 20 bp, synthetic construct
  source 1..20: mol_type = other DNA
gacagagaaatggtgtttaa

SEQ ID NO: 30 - DNA, 21 bp, synthetic construct
  source 1..21: mol_type = other DNA
gagccagattatctctttcta

SEQ ID NO: 31 - DNA, 21 bp, synthetic construct
  source 1..21: mol_type = other DNA
ccattcataataaaatgctaa

SEQ ID NO: 32 - DNA, 21 bp, synthetic construct
  source 1..21: mol_type = other DNA
cggaagacattagggatattt

SEQ ID NO: 33 - DNA, 21 bp, synthetic construct
  source 1..21: mol_type = other DNA
tggaaccatgaaccaagtaaa

SEQ ID NO: 34 - DNA, 21 bp, synthetic construct
  source 1..21: mol_type = other DNA
cccaactgcatcccttgtaaa

SEQ ID NO: 35 - DNA, 21 bp, synthetic construct
  source 1..21: mol_type = other DNA
gcgtcgtgactgggaaaaccc

SEQ ID NO: 36 - DNA, 20 bp, synthetic construct
  source 1..20: mol_type = other DNA
acctgcatccagccatccca

SEQ ID NO: 37 - DNA, 20 bp, synthetic construct
  source 1..20: mol_type = other DNA
cacctagcctcgcacgaact